(19) United States
(12) Patent Application Publication
(10) Pub. No.: US 2010/0167931 A1
(43) Pub. Date: Jul. 1, 2010

(54) HETEROCYCLICALLY SUBSTITUTED HETEROCYCLYLCARBOXYLIC ACID DERIVATIVES

(75) Inventors: Michael Müller, Dusseldorf (DE); Welf-Burkhard Wiese, Langenfeld (DE); Thomas Schenke, Bergisch-Gladbach (DE); Hans-Georg Schwarz, Langenfeld (DE); Heinz-Jûrgen Wroblowsky, Langenfeld (DE); Reiner Fischer, Monheim (DE); Eva-Maria Franken, Limonest (FR); Olga Malsam, Rosrath (DE); Christian Arnold, Langenfeld (DE); Kerstin Ilg, Koln (DE); Arnd Voerste, Koln (DE); Ulrich Görgens, Ratingen (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/663,218

(22) PCT Filed: May 31, 2008
(86) PCT No.: PCT/EP2008/004352

§ 371 Date: Dec. 4, 2009

(30) Foreign Application Priority Data
Jun. 13, 2007 (EP) 07110228.9

(51) Int. Cl. A01N 43/90; C07D 487/04; A01P 3/00; A01P 13/00; A01P 1/00; A01P 7/04; C07D 233/60

(52) U.S. Cl. 504/136; 544/281; 514/259.3; 424/84; 548/341.5

(57) ABSTRACT

The present invention relates to heterocyclic carboxylic acid derivatives of the formula (I) below

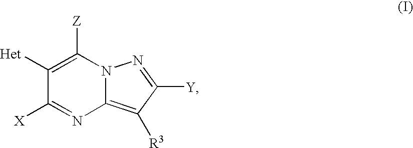

to a process for their preparation, to the use of the heterocyclic carboxylic acid derivatives according to the invention for controlling animal pests and/or phytopathogenic harmful fungi, to methods and compositions for controlling animal pests and/or phytopathogenic harmful fungi in and/or on plants or in and/or on seed of plants, to processes for preparing such compositions and to treated seed and also to the use thereof for controlling pests and/or phytopathogenic harmful fungi in agriculture, horticulture and forestry, in animal health, in the protection of materials and in the domestic and hygiene field.

The present invention relates to heterocyclic carboxylic acid derivatives of the formula (I) below

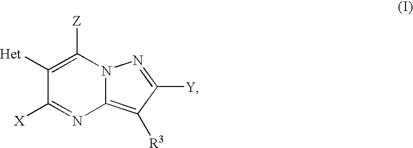

in which the symbols have the meanings given in the description, or to their agrochemically active salts or to mixtures of these compounds and/or their agrochemically active salts with other active compounds for controlling animal pests and/or phytopathogenic harmful fungi, to methods and compositions for controlling animal pests and/or phytopathogenic harmful fungi in and/or on plants or in and/or on seed of plants, to processes for preparing such compositions and to treated seed and to the use thereof for controlling pests and/or phytopathogenic harmful fungi in agriculture, horticulture and forestry, in animal health, in the protection of materials and in the domestic and hygiene field. The present invention furthermore relates to a process for preparing heterocyclic carboxylic acid derivatives of the formula (I).

The present invention furthermore relates to enhancing the activity of crop protection compositions comprising in particular heterocyclic carboxylic acid derivatives of the formula (I) by addition of ammonium or phosphonium salts and, if appropriate, penetrants, to the corresponding compositions, to processes for their preparation and to their use in crop protection as insecticides and/or acaricides and/or fungicides.

It is already known that certain pyrazolopyrimidine derivatives have fungicidal properties, see, for example, WO 04/000844, WO 05/082907 and WO 06/087120. Furthermore, it is already known that certain pyrazolopyrimidine derivatives have insecticidal properties, see, for example, WO 04/000844.

Since the environmental and economic requirements imposed on modern-day pesticides are continually increasing, with regard, for example, to the spectrum of action, toxicity, selectivity, application rate, formation of residues, and favourable preparability, and since, furthermore, there may be problems, for example, with resistances, a constant task is to develop novel pesticides which in some areas at least have advantages over their known counterparts.

This invention now provides heterocyclic carboxylic acid derivatives of the formula (I)

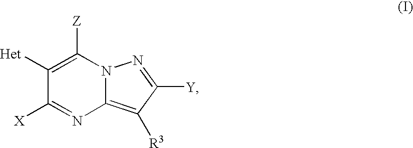

in which the symbols have the following meaning:
X represents hydrogen, halogen, cyano, hydroxyl, optionally substituted alkyl, haloalkyl, optionally substituted alkoxy, optionally substituted phenyl, optionally substituted alkylthio, optionally substituted alkylsulphinyl or optionally substituted alkylsulphonyl;
Het represents one of the radicals

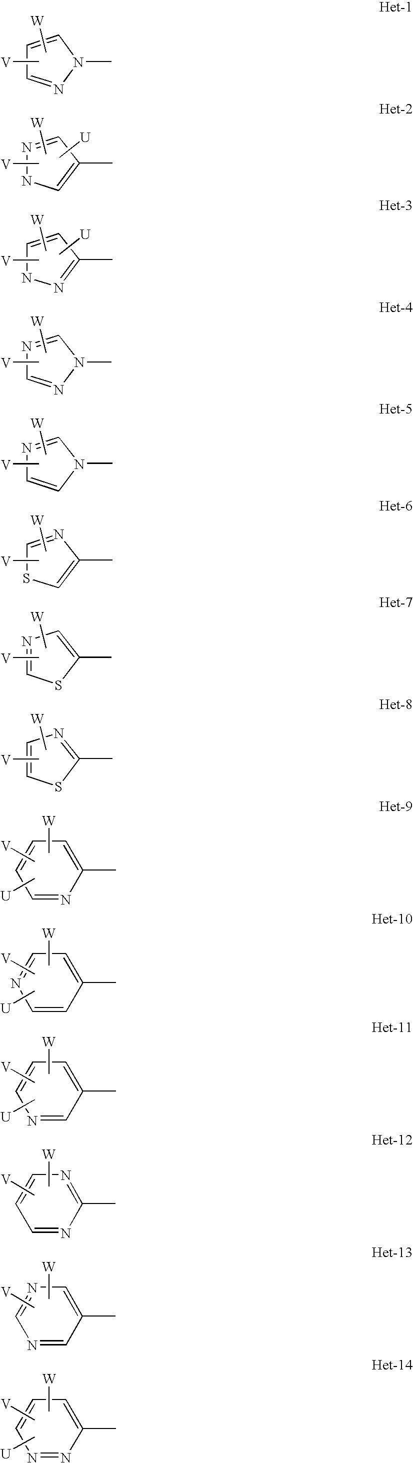

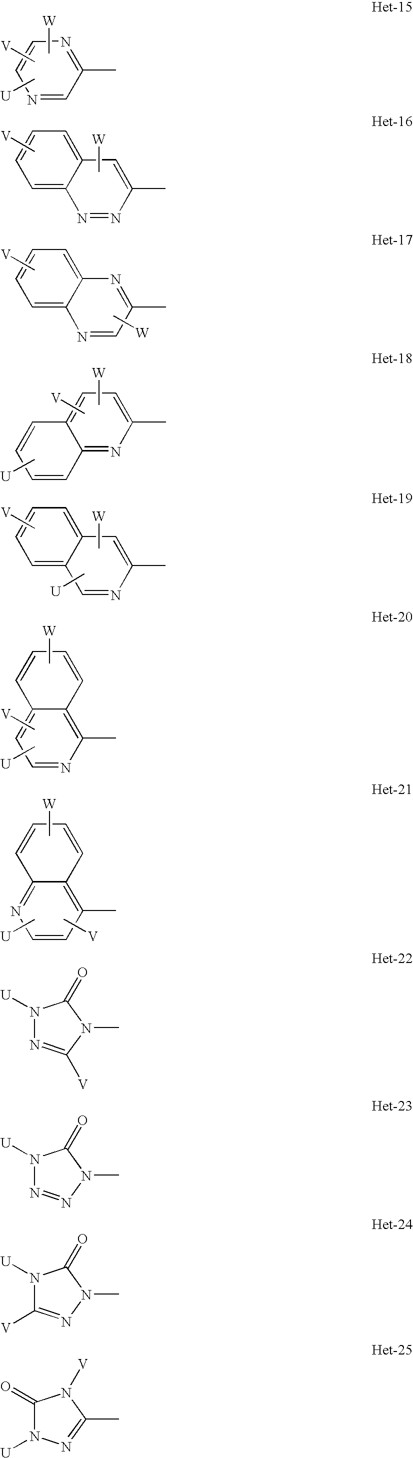

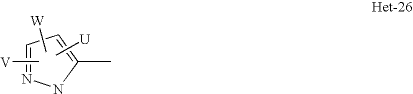

U, V and W independently of one another represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and/or carboxymethyl;
Z represents hydroxyl, halogen or in each case optionally substituted alkoxy, alkylthio, alkylsulphonyl or the group

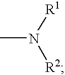

Y represents hydrogen, halogen, optionally substituted alkyl, haloalkyl, or optionally substituted cycloalkyl, O—(C₁-C₄)-alkyl or S(O)₀₋₂(C₁-C₄)-alkyl;
R¹ represents hydrogen, optionally substituted alkyl, optionally substituted alkenyl, optionally substituted alkynyl, haloalkyl, optionally substituted cycloalkyl or optionally substituted heterocyclyl, hydroxyl, optionally substituted alkoxy, amine, optionally substituted alkylamine or optionally substituted dialkylamine;
R² represents hydrogen or alkyl;
or
R¹ and R² together with the nitrogen atom to which they are attached represent an optionally substituted heterocyclic ring;
R³ represents

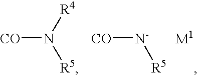

### COOH, CO—O⁻M¹;

R⁴ represents hydrogen, in each case optionally substituted alkyl, alkenyl or alkynyl, haloalkyl, in each case optionally substituted saturated or unsaturated cycloalkyl which may optionally be interrupted by heteroatoms, in each case optionally substituted phenyl, heteroaryl, arylalkyl, heteroarylalkyl;
M¹ represents a cation, such as, for example, mono- or divalent metal atoms, or an optionally alkyl- or arylalkyl-substituted ammonium ion;
R⁵ represents the groups COR⁶, S(O)₁₋₂R⁶, cyano, COOR⁶; or
R⁴ and R⁵ together with the nitrogen atom to which they are attached represent an optionally substituted saturated, unsaturated or aromatic ring which may optionally be interrupted by further heteroatoms;
R⁶ represents hydrogen, in each case optionally substituted alkyl, alkenyl, alkynyl, haloalkyl, represents in each case optionally substituted cycloalkylalkyl, cycloalkyl or cycloalkenyl, in each case optionally substituted aryl, heteroaryl, arylalkyl or heteroarylalkyl;
or
R⁴ and R⁶ together with the N—CO or N—S(O)₁₋₂ group to which they are attached form a 4- to 8-membered cycle which may contain one or more heteroatoms from the group consisting of sulphur, oxygen and/or nitrogen, where oxygen atoms must not be adjacent;
   and agrochemically active salts thereof. The radicals referred to above as optionally substituted, including groups and rings or cycles, can be unsubstituted or substituted, in particular by the same substituents as defined for the corresponding radicals in the further embodiments of the present invention described below. Here, the substituents of one embodiment may be combined as desired with those of another embodiment.

Heterocyclic carboxylic acid derivatives of the formula (I) according to the invention and their agrochemically active salts are highly suitable for use as pesticides, in particular for controlling animal pests, such as insects, parasites of the sub-class of the Acari (Acarina) (such as mites, spider mites and/or ticks) and/or nematodes. They are also suitable for controlling phytopathogenic harmful fungi. The compounds according to the invention mentioned above show in particular strong insecticidal and/or acaricidal and/or nematicidal and/or fungicidal activity and can be used both in crop protection, in the domestic and hygiene field and in the protection of materials.

The compounds of the formula (I) may be present either in pure form or as mixtures of various possible isomeric forms, in particular of stereoisomers, such as E and Z, threo and erythro, and also optical isomers, such as R and S isomers or atropisomers, and, if appropriate, also of tautomers. The invention comprises both the pure isomers and mixtures thereof.

If appropriate, the compounds of the formula (I) may be present in various polymorphic forms or as mixtures of differing polymorphic forms. Both the pure polymorphs and the polymorph mixtures are provided by the invention and can be used according to the invention.

Depending on the nature of the substituents defined above, the compounds of the formula (I) have acidic or basic properties and may form salts, if appropriate also inner salts. If the compounds of the formula (I) carry hydroxyl groups, carboxyl groups or other groups which induce acidic properties, these compounds can be converted with bases into salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl radicals, mono-, di- and trialkanolamines of (C₁-C₄)-alkanols, choline and also chlorocholine. If the compounds of the formula (I) carry amino groups, alkylamino groups or other groups which induce basic properties, these compounds can be converted with acids into salts. Suitable acids are, for example, mineral acids, such as hydrochloric acid, sulphuric acid and phosphoric acid, organic acids, such as acetic acid or oxalic acid, and acidic salts, such as NaHSO₄ and KHSO₄. The salts obtainable in this manner also have insecticidal and/or fungicidal properties.

The terms below are defined as follows in this application:

Optionally substituted groups may be mono- or polysubstituted, where in the case of polysubstitution the substituents may be identical or different.

“Alkyl” is defined as branched or straight-chain C₁₋₁₀-alkyl, such as, for example, methyl, ethyl, n- or isopropyl, n-, iso-, sec- or tert-butyl, n-pentyl, n-hexyl, n-heptyl, n-octyl, n-nonyl, n-decyl and the like, preferably as C₁₋₈-alkyl, particularly preferably as C₁₋₄-alkyl.

“Alkenyl” is defined as branched or straight-chain C₃₋₁₀-alkenyl which contains at least one double bond, for example vinyl, allyl, 1-propenyl, isopropenyl, 1-butenyl, 2-butenyl, 3-butenyl, 1,3-butanedienyl, 1-pentenyl, 2-pentenyl, 3-pentenyl, 4-pentenyl, 1,3 pentanedienyl, 1-hexenyl, 2-hexenyl, 3-hexenyl, 4-hexenyl, 5-hexenyl, 1,4-hexanedienyl and the like, preferably as C₃₋₈-alkenyl, particularly preferably as C₃₋₆-alkenyl.

“Alkynyl” is defined as C₃₋₁₀-alkynyl which contains at least one triple bond and, if appropriate, one or more double bonds, such as, for example, ethynyl, 1-propinyl, propargyl and the like, preferably as C₃₋₈-alkynyl, particularly preferably as C₃₋₆-alkynyl.

Each alkyl moiety in “alkoxy”, “haloalkyl”, “alkoxyalkyl”, “alkylaminocarbonyl”, “dialkylaminoalkyl”, “haloalkoxy”, “alkylcarbonyl”, “alkoxycarbonyl” and “alkoxycarbonylalkyl” and the like refers to the description of “alkyl” given above, including the examples mentioned.

“Cycloalkyl” is defined as C₃₋₁₀-cycloalkyl, such as, for example, cyclopropyl, cyclobutyl, cyclopentyl, cyclohexyl, cycloheptyl, cyclooctyl and the like, preferably as C₃₋₈-cycloalkyl.

“Heterocycloalkyl” is defined as cycloalkyl which contains a 3- to 10-membered ring, where the ring contains one, two or more heteroatoms, such as nitrogen, oxygen and/or sulphur, such as, for example, tetrahydrofuran, thiolane, pyrrolidine, oxathiolane, oxazolidine, tetrahydropyran, piperidine, dioxane and the like; preference is given to tetrahydrofuran.

“Aryl” is defined as unsaturated C₅-C₁₂-cycloalkyl, such as, for example, phenyl, α- or β-naphthyl, preferably phenyl.

“Heteroaryl” is defined as aryl which contains a 3- to 10-membered ring, where the ring contains one, two or more heteroatoms, such as nitrogen, oxygen and/or sulphur, such as, for example, furan, thiophene, pyrrole, oxazole, thiazole, pyrazole, pyridine, pyrimidine, pyrazole and the like.

“Arylalkyl” is defined, for example, as benzyl, phenethyl or α-methylbenzyl; preference is given to benzyl.

“Halogen” is defined as fluorine, chlorine, bromine or iodine; preference is given to fluorine or chlorine.

The formula (I) provides a general definition of the compounds according to the invention, i.e. the heterocyclic carboxylic acid derivatives.

Accordingly, the present invention relates to
(1) chemical compounds, i.e. heterocyclic carboxylic acid derivatives of the formula (I) below

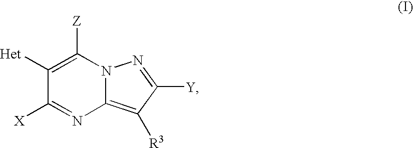

in which
X represents hydrogen, halogen, cyano, hydroxyl, optionally substituted alkyl, haloalkyl, optionally substituted alkoxy, optionally substituted phenyl, optionally substituted alkylthio, optionally substituted alkylsulphinyl or optionally substituted alkylsulphonyl;
Het represents one of the radicals

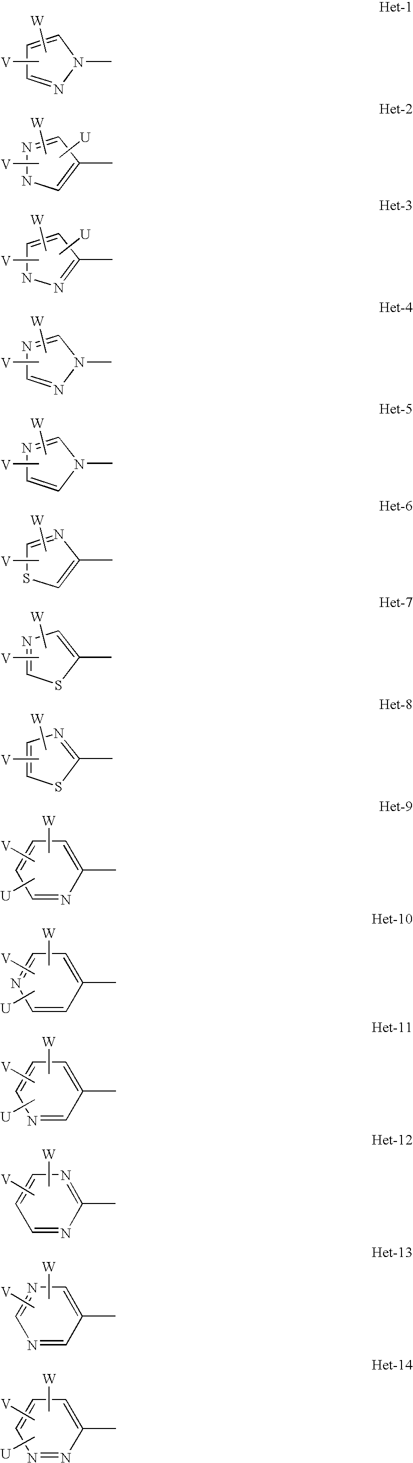

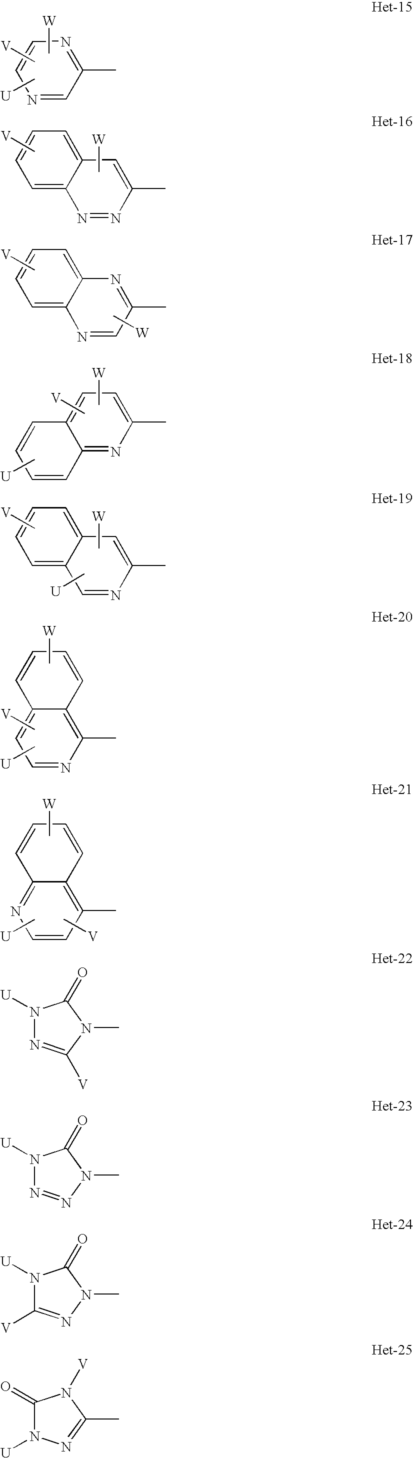

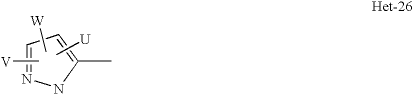

U, V and W independently of one another represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and/or carboxymethyl;
Z represents hydroxyl, halogen or in each case optionally substituted alkoxy, alkylthio, alkylsulphonyl or the group

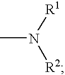

Y represents hydrogen, halogen, optionally substituted alkyl, haloalkyl, or optionally substituted cycloalkyl, O—(C₁-C₄)-alkyl or S(O)₀₋₂(C₁-C₄)-alkyl;
R¹ represents hydrogen, optionally substituted alkyl, optionally substituted alkenyl, optionally substituted alkynyl, haloalkyl, optionally substituted cycloalkyl or optionally substituted heterocyclyl, hydroxyl, optionally substituted alkoxy, amine, optionally substituted alkylamine or optionally substituted dialkylamine;
R² represents hydrogen or alkyl;
or
R¹ and R² together with the nitrogen atom to which they are attached represent an optionally substituted heterocyclic ring;
R³ represents

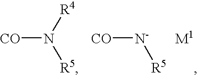

### COOH, CO—O⁻M¹;

R⁴ represents hydrogen, in each case optionally substituted alkyl, alkenyl or alkynyl, haloalkyl, in each case optionally substituted saturated or unsaturated cycloalkyl which may optionally be interrupted by heteroatoms, in each case optionally substituted phenyl, heteroaryl, arylalkyl, heteroarylalkyl;
M¹ represents a cation, such as, for example, mono- or divalent metal atoms, or an optionally alkyl- or arylalkyl-substituted ammonium ion;
R⁵ represents the groups COR⁶, S(O)₁₋₂R⁶, cyano, COOR⁶; or
R⁴ and R⁵ together with the nitrogen atom to which they are attached represent an optionally substituted saturated, unsaturated or aromatic ring which may optionally be interrupted by further heteroatoms;
R⁶ represents hydrogen, in each case optionally substituted alkyl, alkenyl, alkynyl, haloalkyl, represents in each case optionally substituted cycloalkylalkyl, cycloalkyl or cycloalkenyl, in each case optionally substituted aryl, heteroaryl, arylalkyl or heteroarylalkyl;
or
R⁴ and R⁶ together with the N—CO or N—S(O)₁₋₂ group to which they are attached form a 4- to 8-membered cycle which may contain one or more heteroatoms from the group consisting of sulphur, oxygen and/or nitrogen, where oxygen atoms must not be adjacent;
   and agrochemically active salts thereof for controlling animal pests and/or phytopathogenic harmful fungi;
(2) a mixture comprising at least one of the compounds defined under (1) and/or at least one agrochemically active salt thereof and a further active compound selected from the group consisting of insecticides, attractants, sterilants, bactericides, acaricides, nematicides, fungicides, growth-regulators, herbicides, safeners, fertilizers and semiochemicals for controlling animal pests and/or phytopathogenic harmful fungi;
(3) a composition for controlling animal pests and/or phytopathogenic harmful fungi in and/or on plants or in and/or on seed of plants, where the composition mentioned comprises at least one compound as defined under (1) and/or at least one agrochemically active salt thereof or a mixture as defined under (2) and agrochemically customary auxiliaries and/or additives;
(4) the enhancement of the activity against animal pests of compounds as defined under (1) and/or at least one of their agrochemically active salts or a mixture as defined under (2) or a composition as defined under (3) by addition of ammonium or phosphonium salts and, if appropriate, penetrants, and the corresponding compositions;
(5) a method for controlling animal pests and/or phytopathogenic harmful fungi in and/or on plants or in and/or on seed of plants, which method comprises bringing the pests and/or harmful fungi mentioned directly or indirectly into contact with at least one compound as defined under (1) and/or at least one of its agrochemically active salts or a mixture as defined under (2) or a composition as defined under (3) and (4);
(6) a process for preparing a composition as defined under (3) and (4), which process comprises mixing at least one compound as defined under (1) and/or at least one of its agrochemically active salts or a mixture as defined under (2) with agrochemically customary auxiliaries and/or additives;
(7) seed treated with at least one compound as defined under (1) and/or at least one of its agrochemically active salts or a mixture as defined under (2) or a composition as defined under (3) and (4);
(8) intermediates for preparing compounds as defined under (1); and
(9) processes for preparing compounds as defined under (1) and intermediates thereof.

Preferred substituents, fragments and ranges of the radicals listed in the formulae mentioned above and below are illustrated below for a preferred embodiment of the present invention:
X preferably represents hydrogen, fluorine, chlorine, bromine, cyano, hydroxyl, alkoxy having 1 to 4 carbon atoms or alkythio having 1 to 4 carbon atoms, C₁-C₆-alkyl or C₁-C₄-haloalkyl having 1 to 9 halogen atoms;
Het preferably represents one of the radicals

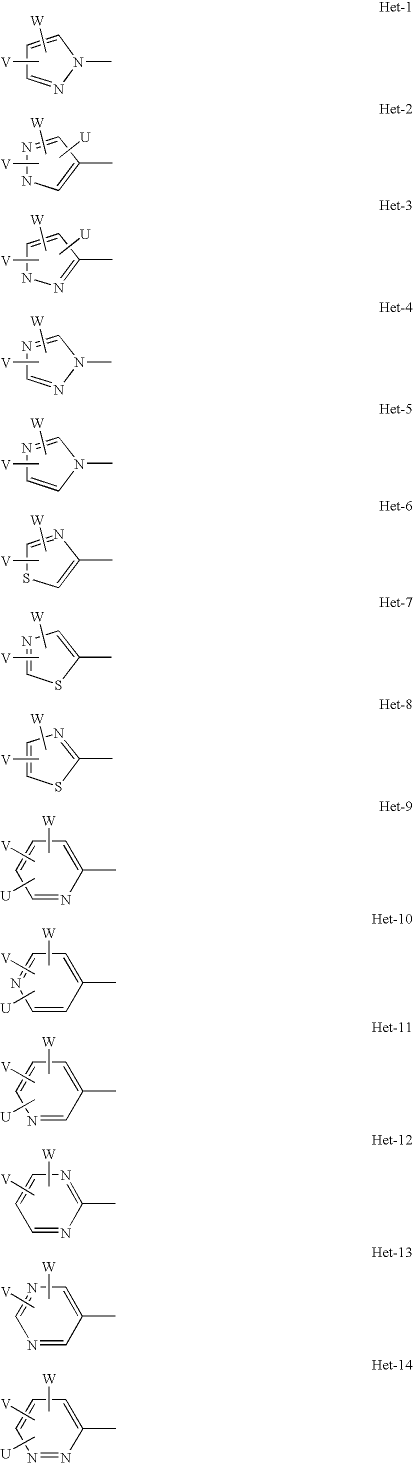

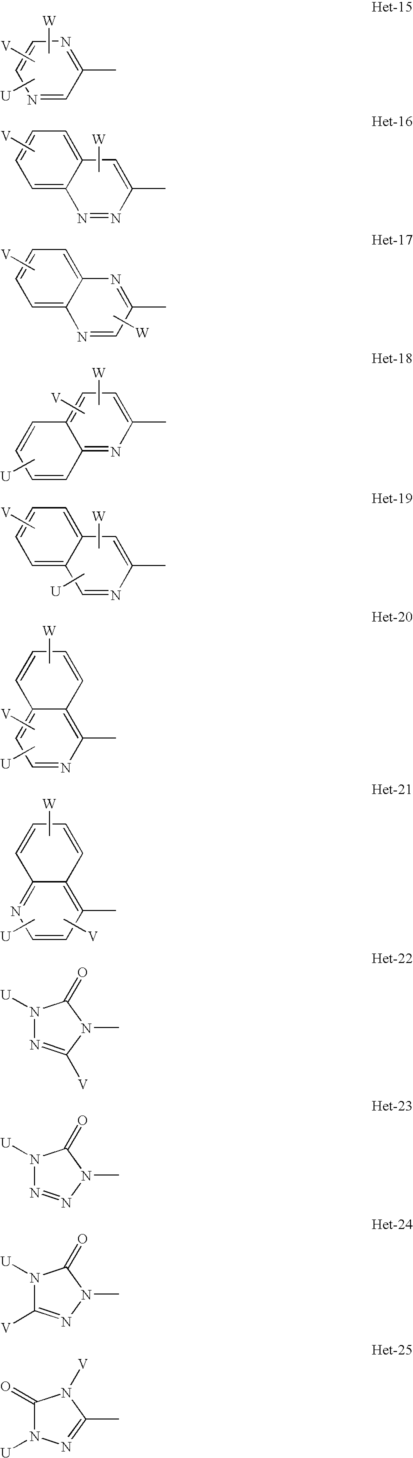

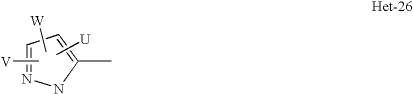

U, V and W independently of one another preferably represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and/or carboxymethyl;
Z preferably represents hydroxyl, chlorine, bromine, represents straight-chain or branched C₁-C₆-alkoxy, C₁-C₆-alkylthio, C₁-C₆-alkylsulphonyl, each of which is optionally substituted by 1 to 7 halogen atoms, or preferably represents the group

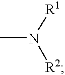

Y preferably represents hydrogen, halogen, C₁-C₄-alkyl which is optionally substituted by 1 to 9 halogen atoms, C₃-C₆-cycloalkyl which is optionally substituted by 1 to 9 halogen atoms, SCH₃, SC₂H₅, SOCH₃, SOC₂H₅, SO₂CH₃, SO₂C₂H₅, OCH₃ or OC₂H₅;
R¹ preferably represents hydrogen, alkyl having 1 to 10 carbon atoms which is unsubstituted or mono- to pentasubstituted by identical or different substituents from the group consisting of cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ preferably represents alkenyl having 3 to 10 carbon atoms which is unsubstituted or mono-to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ preferably represents alkynyl having 3 to 10 carbon atoms which is unsubstituted or mono-to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ preferably represents haloalkyl having 1 to 10 carbon and 1 to 21 halogen atoms; or
R¹ preferably represents cycloalkyl having 3 to 10 carbon atoms which is unsubstituted or mono- to trisubstituted by identical or different substituents from the group consisting of halogen and alkyl, haloalkyl having in each case 1 to 4 carbon atoms; or

R¹ preferably represents saturated or unsaturated heterocyclyl having 3 to 10 ring members and 1 to 3 heteroatoms, such as nitrogen, oxygen and/or sulphur, where the heterocyclyl is unsubstituted or mono- or polysubstituted by halogen, alkyl having 1 to 4 carbon atoms, alkoxyl having 1 to 4 carbon atoms, cyano, nitro, cycloalkyl having 3 to 6 carbon atoms, hydroxyl, alkoxy, alkenyloxy, alkynyloxy having 1 to 6 carbon atoms or mercapto;
R² preferably represents hydrogen or alkyl having 1 to 6 carbon atoms; or
R¹ and R² together with the nitrogen atom to which they are attached preferably represent a saturated or unsaturated heterocyclic ring having 3 to 8 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 4 carbon atoms, haloalkyl having 1 to 4 carbon atoms and 1 to 9 fluorine and/or chlorine atoms, hydroxyl, alkoxy having 1 to 4 carbon atoms, haloalkoxy having 1 to 4 carbon atoms and 1 to 9 fluorine and/or chlorine atoms, mercapto, thioalkyl having 1 to 4 carbon atoms and/or haloalkylthio having 1 to 4 carbon atoms and 1 to 9 fluorine and/or chlorine atoms;
R³ preferably represents

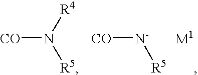

### COOH, CO—O⁻M¹;

R⁴ preferably represents hydrogen,
   preferably represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—O—C₁-C₄-alkyl, preferably represents C₃-C₈-cycloalkyl which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and which may optionally be interrupted by an oxygen or sulphur atom,
   preferably represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
M¹ preferably represents cations from the group of the alkali metals or alkaline earth metals or ammonium ions NH₄, mono-(C₁-C₁₀)-alkylammonium, di-(C₁-C₁₀)-alkylammonium, tri-(C₁-C₁₀)-alkylammonium, tetra-(C₁-C₁₀)-alkylammonium, where the alkyl radicals of the ammonium ions may be substituted by aryl or hydroxyl, or cholinium;
R⁵ preferably represents the groups CO—R⁶, S(O)₁₋₂R⁶, cyano, COOR⁶;
R⁴ and R⁵ together with the nitrogen atom to which they are attached preferably represent a saturated or unsaturated 5- or 6-membered ring which is optionally mono- or polysubstituted by C₁-C₄-alkyl and which may optionally be interrupted by oxygen or sulphur;
R⁶ preferably represents hydrogen, preferably represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, cyano, CO₂—H or CO—O—C₁-C₄-alkyl, preferably represents C₁-C₁₀-haloalkyl having 1 to 21 fluorine and/or chlorine atoms, preferably represents C₃-C₈-cycloalkyl, C₃-C₆-cycloalkyl-C₁-C₂-alkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and which may optionally be interrupted by an oxygen or sulphur atom,
   preferably represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
   or
   R⁴ and R⁶ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent.

The formula (I) provides a general definition of the compounds according to the invention. Particularly preferred substituents, fragments or ranges of the radicals listed in the formulae mentioned above and below are illustrated below for a particularly preferred embodiment of the present invention:
X particularly preferably represents hydrogen, fluorine, chlorine, bromine, cyano, C₁-C₄-alkyl or C₁-C₂-haloalkyl which may be substituted by one to five fluorine and/or chlorine atoms;
Het particularly preferably represents one of the radicals

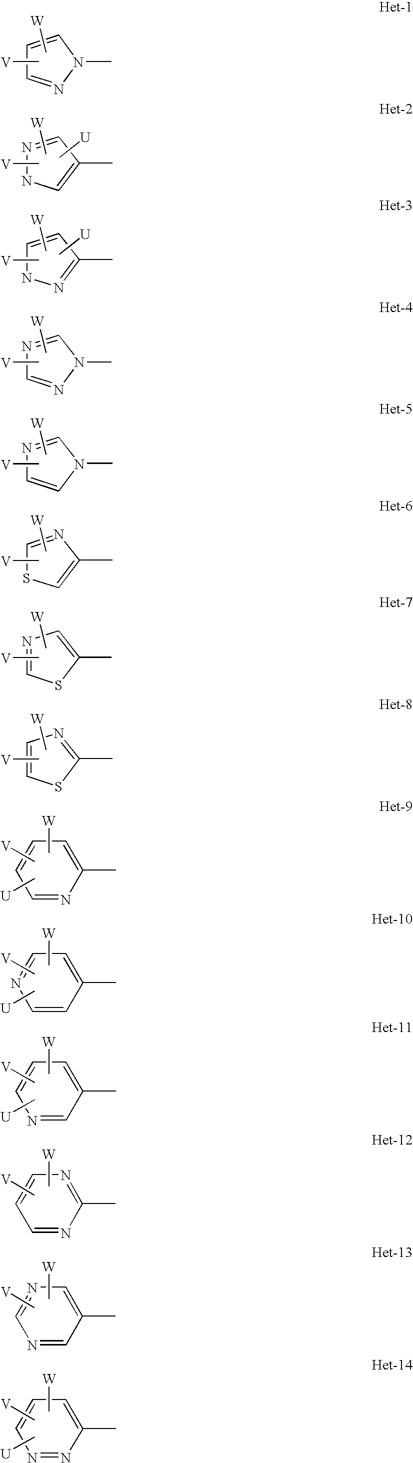

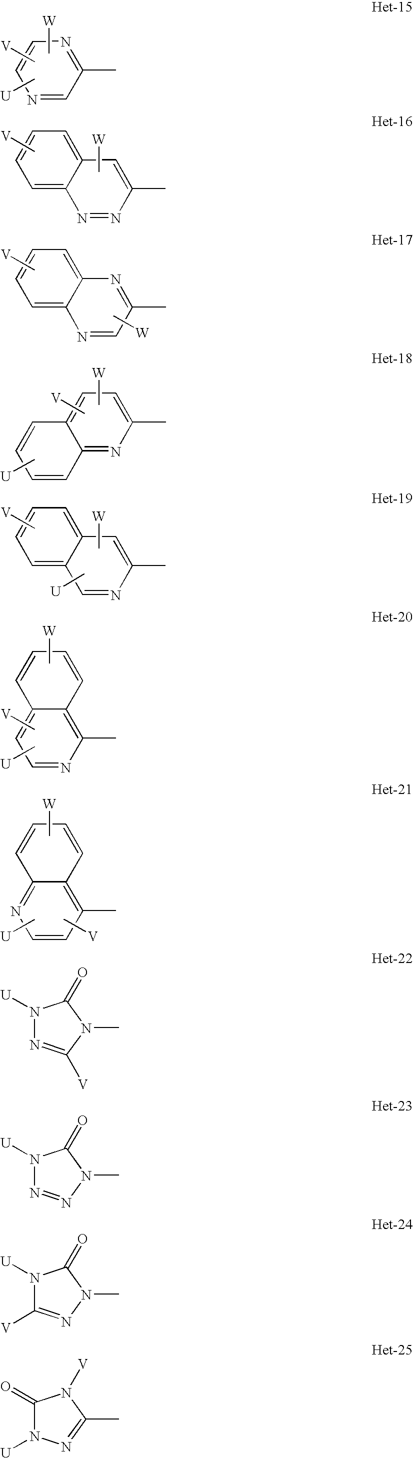

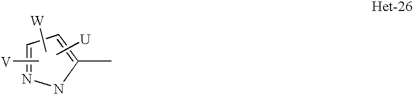

U, V and W independently of one another particularly preferably represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and/or carboxymethyl;
Z particularly preferably represents chlorine, bromine, particularly preferably represents straight-chain or branched C₁-C₄-alkoxy, C₁-C₄-alkylthio, each of which is optionally substituted by 1 to 3 halogen atoms, or particularly preferably represents the group

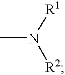

Y particularly preferably represents hydrogen, halogen, (C₁-C₄)-alkyl which is optionally substituted by 1 to 9 halogen atoms, cyclopropyl which is optionally substituted by 1 to 5 halogen atoms; SCH₃, SC₂H₅, SOCH₃, SOC₂H₅, SO₂CH₃, SO₂C₂H₅, OCH₃ or OC₂H₅;
R¹ particularly preferably represents hydrogen, alkyl having 1 to 8 carbon atoms which is unsubstituted or mono- to trisubstituted by identical or different substituents from the group consisting of cyano, alkoxy having 1 to 3 carbon atoms, cycloalkyl having 3 to 6 carbon atoms, alkylthio having 1 to 3 carbon atoms; or
R¹ particularly preferably represents alkenyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ particularly preferably represents alkynyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ particularly preferably represents cycloalkyl having 3 to 8 carbon atoms which is unsubstituted or mono- to trisubstituted by identical or different substituents from the group consisting of fluorine, chlorine and alkyl, haloalkyl, alkoxy having in each case 1 or 2 carbon atoms; or
R¹ particularly preferably represents haloalkyl having 1 to 8 carbon and 1 to 17 fluorine and/or chlorine atoms; or
R¹ particularly preferably represents saturated or unsaturated heterocyclyl having 3 to 8 ring members and 1 or 2 heteroatoms, such as nitrogen, oxygen and/or sulphur, where the heterocyclyl is unsubstituted or mono- or disubstituted by hydroxyl, fluorine, chlorine, alkyl having 1 to 3 carbon atoms, alkoxy having 1 to 3 carbon atoms, cyano, nitro or cycloalkyl having 3 to 6 carbon atoms;
R² particularly preferably represents hydrogen or alkyl having 1 to 6 carbon atoms; or
R¹ and R² together with the nitrogen atom to which they are attached particularly preferably represent a saturated or unsaturated heterocyclic ring having 3 to 6 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 3 carbon atoms, haloalkyl having 1 to 3 carbon atoms and 1 to 5 fluorine and/or chlorine atoms, alkoxy having 1 to 3 carbon atoms, haloalkoxy having 1 to 3 carbon atoms and 1 to 5 fluorine and/or chlorine atoms, thioalkyl having 1 to 3 carbon atoms and/or haloalkylthio having 1 to 3 carbon atoms and 1 to 5 fluorine and/or chlorine atoms;
R³ particularly preferably represents

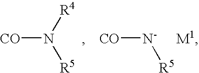

### COOH, CO—O⁻M¹;

R⁴ particularly preferably represents hydrogen,
   particularly preferably represents C₁-C₈-alkyl, C₃-C₈-alkenyl or C₃-C₈-alkynyl, each of which is optionally mono- to trisubstituted by fluorine, chlorine, C₁-C₃-alkoxy, C₁-C₃-alkylthio, cyano, CO₂H or CO—O—C₁-C₃-alkyl, particularly preferably represents C₃-C₆-cycloalkyl, C₃-C₆-cycloalkylmethyl, each of which is optionally mono- to trisubstituted by fluorine, chlorine, C₁-C₃-alkyl, C₁-C₂-haloalkyl, C₁-C₃-alkoxy, CN, CO₂H or CO—O—C₁-C₃-alkyl and which may optionally be interrupted by an oxygen or sulphur atom,
   particularly preferably represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
M¹ particularly preferably represents Na⁺, K⁺, ½ Ca²⁺, ½Mg²⁺, NH₄⁺, NH₃CH₃⁺, NH₂(CH₃)₂⁺, NH(CH₃)₃⁺, NH(C₂H₅)₃⁺NH₂(C₂H₅)₂⁺, NH₃C₂H₅⁺, NH₃i-C₃H₇⁺, NH₂(i-C₃H₇)₂⁺, NH₃—CH₂—C₆H₅⁺,

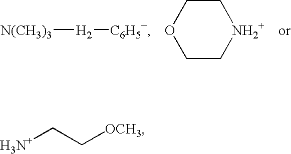

R⁵ particularly preferably represents the groups COR⁶, S(O)₁₋₂R⁶, cyano, COOR⁶;
R⁴ and R⁵ together with the nitrogen atom to which they are attached particularly preferably represent a saturated or unsaturated 5- or 6-membered ring which is optionally mono- or disubstituted by C₁-C₂-alkyl and which may optionally be interrupted by oxygen or sulphur;
R⁶ particularly preferably represents hydrogen, particularly preferably represents C₁-C₈-alkyl, C₃-C₈-alkenyl or C₃-C₈-alkynyl, each of which is optionally mono- to pentasubstituted by fluorine, chlorine, C₁-C₃-alkoxy, C₁-C₃-alkylthio, cyano, CO₂H or CO—O—C₁-C₃-alkyl, particularly preferably represents C₁-C₈-haloalkyl having 1 to 17 fluorine and/or chlorine atoms, particularly preferably represents C₃-C₆-cycloalkyl, C₃-C₆-cycloalkylmethyl or C₅-C₆-cycloalkenyl, each of which is optionally mono- to trisubstituted by fluorine, chlorine, C₁-C₄-alkyl, C₁-C₂-haloalkyl, C₁-C₂-alkoxy, CN, CO₂H or CO—O—C₁-C₂-alkyl and which may optionally be interrupted by an oxygen or sulphur atom,
   particularly preferably represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
or
R⁴ and R⁶ together with the N—CO or N—S(O)₁₋₂ group to which they are attached form a 4- to 6-membered cycle which may contain one or more heteroatoms from the group consisting of sulphur, oxygen and nitrogen, where oxygen atoms must not be adjacent.

The formula (I) provides a general definition of the compounds according to the invention. Very particularly preferred substituents, fragments or ranges of the radicals listed in the formulae mentioned above and below are illustrated below for a very particularly preferred embodiment of the present invention:
X very particularly preferably represents hydrogen, chlorine, bromine, methyl or trifluoromethyl;
Het very particularly preferably represents one of the radicals

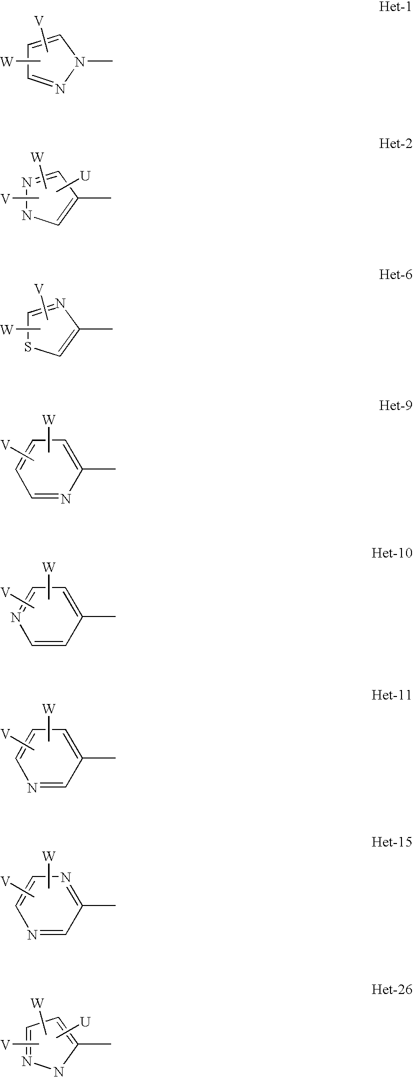

U, V and W independently of one another very particularly preferably represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and/or carboxymethyl;
Z very particularly preferably represents chlorine, methoxy, ethoxy, methylthio, ethylhio or the group

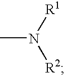

Y very particularly preferably represents hydrogen, chlorine, bromine, alkyl having 1 or 2 carbon atoms, haloalkyl having 1 or 2 carbon and 1 to 5 halogen atoms or cyclopropyl;
R¹ very particularly preferably represents hydrogen, alkyl having 1 to 4 carbon atoms which is unsubstituted or mono- to tetrasubstituted by identical or different substituents from the group consisting of methyl, ethyl, methoxy, ethoxy, cyclopropyl, cyclopentyl, cyclohexyl, methylthio and ethylthio; or
R¹ very particularly preferably represents alkenyl having 3 to 6 carbon atoms which is unsubstituted or mono- to trisubstituted by identical or different substituents from the group consisting of fluorine, chlorine, methoxy, ethoxy, methylthio and ethylthio; or
R¹ very particularly preferably represents alkynyl having 3 to 6 carbon atoms which is unsubstituted or mono- to trisubstituted by identical or different substituents from the group consisting of fluorine, chlorine, methoxy, ethoxy, methylthio and ethylthio; or
R¹ very particularly preferably represents cycloalkyl having 3 to 6 carbon atoms which is unsubstituted or mono- to trisubstituted by identical or different substituents from the group consisting of fluorine, chlorine, methyl, ethyl, methoxy and trifluoromethyl;
R¹ very particularly preferably represents haloalkyl having 1 to 4 carbon atoms which is substituted by 1 to 9 fluorine and/or chlorine atoms;
R² very particularly preferably represents hydrogen, alkyl having 1 or 2 carbon atoms; or
R¹ and R² together with the nitrogen atom to which they are attached very particularly preferably represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ very particularly preferably represents

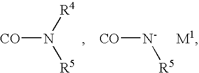

### COOH, CO—O⁻M¹;

R⁴ very particularly preferably represents hydrogen;
M¹ very particularly preferably represents Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺;
R⁵ very particularly preferably represents the groups COR⁶, S(O)R⁶, S(O)₂R⁶, cyano, COOR⁶;
R⁶ very particularly preferably represents hydrogen, very particularly preferably represents alkyl having 1 to 3 carbon atoms or cyclopropyl, each of which is optionally mono- to trisubstituted by methoxy, ethoxy, methylthio, ethylthio, cyano, CO—O-methyl or CO—O-ethyl, very particularly preferably represents haloalkyl having 1 to 3 carbon and 1 to 7 fluorine and/or chlorine atoms.

The formula (I) provides a general definition of the compounds according to the invention. Especially preferred substituents, fragments or ranges of the radicals listed in the formulae mentioned above and below are illustrated below for an especially preferred embodiment of the present invention:
X especially preferably represents hydrogen, chlorine, bromine, methyl or trifluoromethyl;
Het especially preferably represents pyridinyl which is attached in the 2-, 3- or 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   especially preferably represents pyrazinyl which is attached in the 2-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   especially preferably represents pyrazolyl which is attached in the 1-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoro-isopropyl, heptafluoro-n-propyl, carboxyl and carboxymethyl or
   especially preferably represents pyrazolyl which is attached in the 4- or 5-position and may be mono- to trisubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   especially preferably represents thiazolyl which is attached in the 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl;
Z especially preferably represents chlorine, methoxy, ethoxy, methylthio, ethylhio or the group

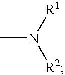

Y especially preferably represents hydrogen, chlorine, bromine, methyl, ethyl, trifluoromethyl or cyclopropyl;
R¹ especially preferably represents hydrogen, alkyl having 1 to 4 carbon atoms which is unsubstituted or mono- to tetrasubstituted by identical or different substituents from the group consisting of methyl, ethyl, methoxy, ethoxy, cyclopropyl, cyclopentyl, cyclohexyl, methylthio and ethylthio; or
R¹ especially preferably represents alkenyl having 3 to 6 carbon atoms which is unsubstituted or mono- to trisubstituted by identical or different substituents from the group consisting of fluorine, chlorine, methoxy, ethoxy, methylthio and ethylthio; or
R¹ especially preferably represents alkynyl having 3 to 6 carbon atoms which is unsubstituted or mono- to trisubstituted by identical or different substituents from the group consisting of fluorine, chlorine, methoxy, ethoxy, methylthio and ethylthio; or
R¹ especially preferably represents cycloalkyl having 3 to 6 carbon atoms which is unsubstituted or mono- to trisubstituted by identical or different substituents from the group consisting of fluorine, chlorine, methyl, ethyl, methoxy and trifluoromethyl;
R¹ especially preferably represents haloalkyl having 1 to 4 carbon atoms which is substituted by 1 to 9 fluorine and/or chlorine atoms;
R² especially preferably represents hydrogen, methyl or ethyl; or
R¹ and R² together with the nitrogen atom to which they are attached especially preferably represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ especially preferably represents

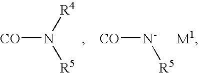

### COOH, COOH, CO—O⁻M¹;

R⁴ especially preferably represents hydrogen;
M¹ especially preferably represents Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺;
R⁵ especially preferably represents S(O)R⁶, S(O)₂R⁶;
R⁶ especially preferably represents hydrogen, especially preferably represents alkyl having 1 to 3 carbon atoms or cyclopropyl, each of which is optionally mono- to trisubstituted by fluorine, chlorine, methoxy, ethoxy, methylthio, ethylthio, cyano, CO—O-methyl or CO—O-ethyl, especially preferably represents haloalkyl having 1-3 carbon and 1 to 7 fluorine and/or chlorine atoms.

Emphasis is given to compounds of the formula (I) in which X represents halogen.

Emphasis is given to compounds of the formula (I) in which Z represents the group

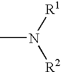

Emphasis is given to compounds of the formula (I) in which Z represents the group

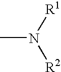

and R² represents hydrogen.

Emphasis is given to compounds of the formula (I) in which Z represents the group

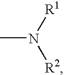

R¹ represents hydrogen, alkyl having 1 to 10 carbon atoms which is unsubstituted or mono- to pentasubstituted by identical or different substituents from the group consisting of cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms and haloalkyl having 1 to 10 carbon and 1 to 21 halogen atoms and R² represents hydrogen.

Emphasis is given to compounds of the formula (I) in which R³ represents COOH or CO—O⁻M¹.

Emphasis is given to compounds of the formula (I), in which R³ represents

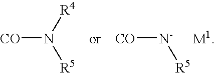

Emphasis is given to compounds of the formula (I) in which R⁴ represents hydrogen.

Emphasis is given to compounds of the formula (I) in which
Het represents pyridinyl which is attached in the 2-, 3- or 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   represents pyrazinyl which is attached in the 2-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   represents pyrazolyl which is attached in the 1-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and carboxymethyl or
   represents pyrazolyl which is attached in the 4- or 5-position and may be mono- to trisubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   represents thiazolyl which is attached in the 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl.

Emphasis is given to compounds of the formula (I) in which
Het represents pyridinyl which is attached in the 2-, 3- or 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and/or carboxymethyl.

Emphasis is given to compounds of the formula (I) in which
Het represents pyrazinyl which is attached in the 2-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl.

Emphasis is given to compounds of the formula (I) in which
Het represents pyrazolyl which is attached in the 1-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and carboxymethyl.

Emphasis is given to compounds of the formula (I) in which
Het represents pyrazolyl which is attached in the 4- or 5-position and may be mono- to trisubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl.

Emphasis is given to compounds of the formula (I) in which
Het represents thiazolyl which is attached in the 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl.

Emphasis is given to compounds of the formula (I) in which R⁵ represents S(O)R⁶ and R⁶ represents alkyl having 1 to 3 carbon atoms and/or haloalkyl having 1 to 3 carbon atoms and 1 to 5 fluorine and/or chlorine atoms and/or cyclopropyl.

Emphasis is given to compounds of the formula (I) in which R⁵ represents S(O)₂R⁶ and R⁶ represents alkyl having 1 to 3 carbon atoms and/or haloalkyl having 1 to 3 carbon atoms and 1 to 5 fluorine and/or chlorine atoms and/or cyclopropyl.

Emphasis is given to compounds of the formula (I) in which
R⁵ represents S(O)R⁶ or S(O)₂R⁶ and R⁶ represents hydrogen, represents C₁-C₈-alkyl, C₃-C₈-alkenyl or C₃-C₈-alkynyl, each of which is optionally mono- to pentasubstituted by fluorine, chlorine, C₁-C₃-alkoxy, C₁-C₃-alkylthio, cyano, CO₂H or CO—O—C₁-C₃-alkyl, represents C₁-C₈-haloalkyl having 1 to 17 fluorine and/or chlorine atoms, represents C₃-C₆-cycloalkyl, C₃-C₆-cycloalkylmethyl or C₅-C₆-cycloalkenyl, each of which is optionally mono- to trisubstituted by fluorine, chlorine, C₁-C₄-alkyl, C₁-C₂-haloalkyl, C₁-C₂-alkoxy, CN, CO₂H or CO—O—C₁-C₂-alkyl and which may optionally be interrupted by an oxygen or sulphur atom.

Emphasis is given to compounds of the formula (I) in which
R⁵ represents S(O)R⁶ or S(O)₂R⁶ and
Het represents pyridinyl which is attached in the 2-, 3- or 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   represents pyrazinyl which is attached in the 2-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   represents pyrazolyl which is attached in the 1-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoro-isopropyl, heptafluoro-n-propyl, carboxyl and carboxymethyl or
   represents pyrazolyl which is attached in the 4- or 5-position and may be mono- to trisubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
   represents thiazolyl which is attached in the 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl.

The general or preferred radical definitions or illustrations listed above can be combined with one another as desired, i.e. including combinations between the respective ranges and preferred ranges. They apply to the end products and, correspondingly, to precursors and intermediates.

Saturated or unsaturated hydrocarbon radicals, such as alkyl or alkenyl, can in each case be straight-chain or branched as far as this is possible, including in combination with heteroatoms, such as, for example, in alkoxy.

Unless indicated otherwise, optionally substituted radicals may be mono- or polysubstituted, where in the case of polysubstitution the substituents can be identical or different.

In addition to the compounds mentioned in the Preparation Examples, specific mention may be made of the following compounds of the formula (I):

TABLE 1

| | | | | (I) |
|---|---|---|---|---|
| 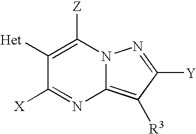 | | | | |
| X | Het | Z | R³ | Y |
| Cl | 3,5-(CH₃)₂-pyrazol-1-yl | NH—CH(CH₃)₂ | CO—NH—SO₂—C₂H₅ | H |
| Cl | 3,5-(CH₃)₂-pyrazol-1-yl | NH—CH(CH₃)₂ | 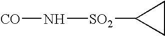 | H |
| Cl | 3,5-(CH₃)₂-pyrazol-1-yl | NH—CHCH₃—CH(CH₃)₂ | CO₂H | H |
| Cl | 3,5-(CH₃)₂-pyrazol-1-yl | NH—CHCH₃—CH(CH₃)₂ | CO—NH—SO₂—CH₃ | H |
| Cl | 3,5-(CH₃)₂-pyrazol-1-yl | NH—CHCH₃—CH(CH₃)₂ | CO—NH—SO₂—C₂H₅ | H |
| Cl | 3,5-(CH₃)₂-pyrazol-1-yl | NH—CHCH₃—CH(CH₃)₂ | 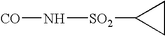 | H |
| Cl | 3,5-(CH₃)₂-pyrazol-1-yl | NH—CH₂—CF₃ | CO₂H | H |
| Cl | 3,5-(CH₃)₂-pyrazol-1-yl | NH—CH₂—CF₃ | CO—NH—SO₂—CH₃ | H |
| Cl | 3,5-(CH₃)₂-pyrazol-1-yl | NH—CH₂—CF₃ | CO—NH—SO₂—C₂H₅ | H |
| Cl | 3,5-(CH₃)₂-pyrazol-1-yl | NH—CH₂—CF₃ | 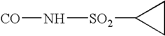 | H |
| Cl | 3,5-(CH₃)₂-pyrazol-1-yl | NH—CH(CH₃)CF₃ | CO₂H | H |
| Cl | 3,5-(CH₃)₂-pyrazol-1-yl | NH—CH(CH₃)CF₃ | CO—NH—SO₂—CH₃ | H |
| Cl | 3,5-(CH₃)₂-pyrazol-1-yl | NH—CH(CH₃)CF₃ | CO—NH—SO₂—C₂H₅ | H |
| Cl | 3,5-(CH₃)₂-pyrazol-1-yl | NH—CH(CH₃)CF₃ | 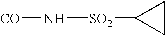 | H |
| Cl | 3,5-(CH₃)₂-pyrazol-1-yl | NH—CH₂—CHF₂ | CO₂H | H |
| Cl | 3,5-(CH₃)₂-pyrazol-1-yl | NH—CH₂—CHF₂ | CO—NH—SO₂—CH₃ | H |
| Cl | 3,5-(CH₃)₂-pyrazol-1-yl | NH—CH₂—CHF₂ | CO—NH—SO₂—C₂H₅ | H |
| Cl | 3,5-(CH₃)₂-pyrazol-1-yl | NH—CH₂—CHF₂ | 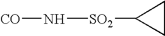 | H |
| Cl | 3,5-(CF₃)₂-pyrazol-1-yl | NH—CH(CH₃)₂ | CO—NH—SO₂—C₂H₅ | H |
| Cl | 3,5-(CF₃)₂-pyrazol-1-yl | NH—CH(CH₃)₂ | 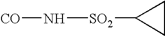 | H |
| Cl | 3,5-(CF₃)₂-pyrazol-1-yl | NH—CHCH₃—CH(CH₃)₂ | CO₂H | H |
| Cl | 3,5-(CF₃)₂-pyrazol-1-yl | NH—CHCH₃—CH(CH₃)₂ | CO—NH—SO₂—CH₃ | H |
| Cl | 3,5-(CF₃)₂-pyrazol-1-yl | NH—CHCH₃—CH(CH₃)₂ | CO—NH—SO₂—C₂H₅ | H |
| Cl | 3,5-(CF₃)₂-pyrazol-1-yl | NH—CHCH₃—CH(CH₃)₂ | 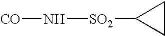 | H |
| Cl | 3,5-(CF₃)₂-pyrazol-1-yl | NH—CH₂—CF₃ | CO—NH—SO₂—C₂H₅ | H |
| Cl | 3,5-(CF₃)₂-pyrazol-1-yl | NH—CH₂—CF₃ | 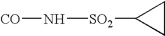 | H |
| Cl | 3,5-(CF₃)₂-pyrazol-1-yl | NH—CH(CH₃)CF₃ | CO₂H | H |
| Cl | 3,5-(CF₃)₂-pyrazol-1-yl | NH—CH(CH₃)CF₃ | CO—NH—SO₂—CH₃ | H |
| Cl | 3,5-(CF₃)₂-pyrazol-1-yl | NH—CH(CH₃)CF₃ | CO—NH—SO₂—C₂H₅ | H |
| Cl | 3,5-(CF₃)₂-pyrazol-1-yl | NH—CH(CH₃)CF₃ | 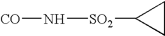 | H |
| Cl | 3,5-(CF₃)₂-pyrazol-1-yl | NH—CH₂—CHF₂ | CO₂H | H |
| Cl | 3,5-(CF₃)₂-pyrazol-1-yl | NH—CH₂—CHF₂ | CO—NH—SO₂—CH₃ | H |
| Cl | 3,5-(CF₃)₂-pyrazol-1-yl | NH—CH₂—CHF₂ | CO—NH—SO₂—C₂H₅ | H |
| Cl | 3,5-(CF₃)₂-pyrazol-1-yl | NH—CH₂—CHF₂ | 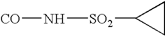 | H |
| Cl | 4-Cl-pyrazol-1-yl | NH—CH(CH₃)₂ | 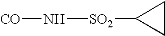 | H |
| Cl | 4-Cl-pyrazol-1-yl | NH—CHCH₃—CH(CH₃)₂ | CO—NH—SO₂—C₂H₅ | H |
| Cl | 4-Cl-pyrazol-1-yl | NH—CHCH₃—CH(CH₃)₂ | 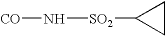 | H |
| Cl | 4-Cl-pyrazol-1-yl | NH—CH₂—CF₃ | CO₂H | H |
| Cl | 4-Cl-pyrazol-1-yl | NH—CH₂—CF₃ | CO—NH—SO₂—CH₃ | H |
| Cl | 4-Cl-pyrazol-1-yl | NH—CH₂—CF₃ | CO—NH—SO₂—C₂H₅ | H |
| Cl | 4-Cl-pyrazol-1-yl | NH—CH₂—CF₃ | 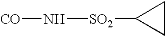 | H |
| Cl | 4-Cl-pyrazol-1-yl | NH—CH(CH₃)CF₃ | CO₂H | H |
| Cl | 4-Cl-pyrazol-1-yl | NH—CH(CH₃)CF₃ | CO—NH—SO₂—CH₃ | H |
| Cl | 4-Cl-pyrazol-1-yl | NH—CH(CH₃)CF₃ | CO—NH—SO₂—C₂H₅ | H |
| Cl | 4-Cl-pyrazol-1-yl | NH—CH(CH₃)CF₃ | 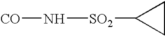 | H |
| Cl | 4-Cl-pyrazol-1-yl | NH—CH₂—CHF₂ | CO₂H | H |
| Cl | 4-Cl-pyrazol-1-yl | NH—CH₂—CHF₂ | CO—NH—SO₂—CH₃ | H |
| Cl | 4-Cl-pyrazol-1-yl | NH—CH₂—CHF₂ | CO—NH—SO₂—C₂H₅ | H |
| Cl | 4-Cl-pyrazol-1-yl | NH—CH₂—CHF₂ | 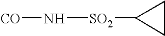 | H |
| Cl | pyrazol-1-yl | NH—CH(CH₃)₂ | CO₂H | H |
| Cl | pyrazol-1-yl | NH—CH(CH₃)₂ | CO—NH—SO₂—CH₃ | H |
| Cl | pyrazol-1-yl | NH—CH(CH₃)₂ | CO—NH—SO₂—C₂H₅ | H |
| Cl | pyrazol-1-yl | NH—CH(CH₃)₂ | 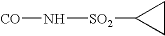 | H |
| Cl | pyrazol-1-yl | NH—CHCH₃—CH(CH₃)₂ | CO₂H | H |
| Cl | pyrazol-1-yl | NH—CHCH₃—CH(CH₃)₂ | CO—NH—SO₂—CH₃ | H |
| Cl | pyrazol-1-yl | NH—CHCH₃—CH(CH₃)₂ | CO—NH—SO₂—C₂H₅ | H |
| Cl | pyrazol-1-yl | NH—CHCH₃—CH(CH₃)₂ | 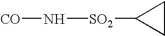 | H |
| Cl | pyrazol-1-yl | NH—CH₂—CF₃ | CO₂H | H |
| Cl | pyrazol-1-yl | NH—CH₂—CF₃ | CO—NH—SO₂—CH₃ | H |
| Cl | pyrazol-1-yl | NH—CH₂—CF₃ | CO—NH—SO₂—C₂H₅ | H |
| Cl | pyrazol-1-yl | NH—CH₂—CF₃ | 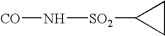 | H |
| Cl | pyrazol-1-yl | NH—CH(CH₃)CF₃ | CO₂H | H |
| Cl | pyrazol-1-yl | NH—CH(CH₃)CF₃ | CO—NH—SO₂—CH₃ | H |
| Cl | pyrazol-1-yl | NH—CH(CH₃)CF₃ | CO—NH—SO₂—C₂H₅ | H |
| Cl | pyrazol-1-yl | NH—CH(CH₃)CF₃ | 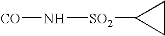 | H |
| Cl | pyrazol-1-yl | NH—CH₂—CHF₂ | CO₂H | H |
| Cl | pyrazol-1-yl | NH—CH₂—CHF₂ | CO—NH—SO₂—CH₃ | H |
| Cl | pyrazol-1-yl | NH—CH₂—CHF₂ | CO—NH—SO₂—C₂H₅ | H |
| Cl | pyrazol-1-yl | NH—CH₂—CHF₂ | 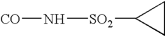 | H |
| Cl | 1,3,5-(CH₃)₃-pyrazol-4-yl | NH—CH(CH₃)CF₃ | CO—NH—SO₂—C₂H₅ | H |
| Cl | 1,3,5-(CH₃)₃-pyrazol-4-yl | NH—CH(CH₃)CF₃ | 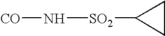 | H |
| Cl | 1,3,5-(CH₃)₃-pyrazol-4-yl | NH—CH(CH₃)₂ | CO₂H | H |
| Cl | 1,3,5-(CH₃)₃-pyrazol-4-yl | NH—CH(CH₃)₂ | CO—NH—SO₂—CH₃ | H |
| Cl | 1,3,5-(CH₃)₃-pyrazol-4-yl | NH—CH(CH₃)₂ | CO—NH—SO₂—C₂H₅ | H |
| Cl | 1,3,5-(CH₃)₃-pyrazol-4-yl | NH—CH(CH₃)₂ | 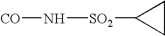 | H |
| Cl | 1,3,5-(CH₃)₃-pyrazol-4-yl | NH—CHCH₃—CH(CH₃)₂ | CO₂H | H |
| Cl | 1,3,5-(CH₃)₃-pyrazol-4-yl | NH—CHCH₃—CH(CH₃)₂ | CO—NH—SO₂—CH₃ | H |
| Cl | 1,3,5-(CH₃)₃-pyrazol-4-yl | NH—CHCH₃—CH(CH₃)₂ | CO—NH—SO₂—C₂H₅ | H |
| Cl | 1,3,5-(CH₃)₃-pyrazol-4-yl | NH—CHCH₃—CH(CH₃)₂ | 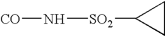 | H |
| Cl | 1,3,5-(CH₃)₃-pyrazol-4-yl | NH—CH₂—CF₃ | CO₂H | H |
| Cl | 1,3,5-(CH₃)₃-pyrazol-4-yl | NH—CH₂—CF₃ | CO—NH—SO₂—CH₃ | H |
| Cl | 1,3,5-(CH₃)₃-pyrazol-4-yl | NH—CH₂—CF₃ | CO—NH—SO₂—C₂H₅ | H |
| Cl | 1,3,5-(CH₃)₃-pyrazol-4-yl | NH—CH₂—CF₃ | 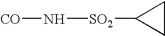 | H |
| Cl | 1,3,5-(CH₃)₃-pyrazol-4-yl | NH—CH₂—CHF₂ | CO₂H | H |
| Cl | 1,3,5-(CH₃)₃-pyrazol-4-yl | NH—CH₂—CHF₂ | CO—NH—SO₂—CH₃ | H |
| Cl | 1,3,5-(CH₃)₃-pyrazol-4-yl | NH—CH₂—CHF₂ | CO—NH—SO₂—C₂H₅ | H |
| Cl | 1,3,5-(CH₃)₃-pyrazol-4-yl | NH—CH₂—CHF₂ | 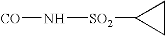 | H |
| Cl | 2,5-(CH₃)₂-thiazol-4-yl | NH—CH(CH₃)₂ | CO—NH—SO₂—C₂H₅ | H |
| Cl | 2,5-(CH₃)₂-thiazol-4-yl | NH—CH(CH₃)₂ | 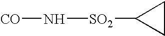 | H |
| Cl | 2,5-(CH₃)₂-thiazol-4-yl | NH—CHCH₃—CH(CH₃)₂ | CO₂H | H |
| Cl | 2,5-(CH₃)₂-thiazol-4-yl | NH—CHCH₃—CH(CH₃)₂ | CO—NH—SO₂—CH₃ | H |
| Cl | 2,5-(CH₃)₂-thiazol-4-yl | NH—CHCH₃—CH(CH₃)₂ | CO—NH—SO₂—C₂H₅ | H |
| Cl | 2,5-(CH₃)₂-thiazol-4-yl | NH—CHCH₃—CH(CH₃)₂ | 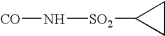 | H |
| Cl | 2,5-(CH₃)₂-thiazol-4-yl | NH—CH₂—CF₃ | CO₂H | H |
| Cl | 2,5-(CH₃)₂-thiazol-4-yl | NH—CH₂—CF₃ | CO—NH—SO₂—CH₃ | H |
| Cl | 2,5-(CH₃)₂-thiazol-4-yl | NH—CH₂—CF₃ | CO—NH—SO₂—C₂H₅ | H |
| Cl | 2,5-(CH₃)₂-thiazol-4-yl | NH—CH₂—CF₃ | 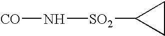 | H |
| Cl | 2,5-(CH₃)₂-thiazol-4-yl | NH—CH(CH₃)CF₃ | CO₂H | H |
| Cl | 2,5-(CH₃)₂-thiazol-4-yl | NH—CH(CH₃)CF₃ | CO—NH—SO₂—CH₃ | H |
| Cl | 2,5-(CH₃)₂-thiazol-4-yl | NH—CH(CH₃)CF₃ | CO—NH—SO₂—C₂H₅ | H |
| Cl | 2,5-(CH₃)₂-thiazol-4-yl | NH—CH(CH₃)CF₃ | 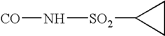 | H |
| Cl | 2,5-(CH₃)₂-thiazol-4-yl | NH—CH₂—CHF₂ | CO₂H | H |
| Cl | 2,5-(CH₃)₂-thiazol-4-yl | NH—CH₂—CHF₂ | CO—NH—SO₂—CH₃ | H |
| Cl | 2,5-(CH₃)₂-thiazol-4-yl | NH—CH₂—CHF₂ | CO—NH—SO₂—C₂H₅ | H |
| Cl | 2,5-(CH₃)₂-thiazol-4-yl | NH—CH₂—CHF₂ | 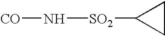 | H |
| Cl | 3,5-F₂-pyridin-2-yl | NH—CH(CH₃)₂ | CO—NH—SO₂—C₂H₅ | H |
| Cl | 3,5-F₂-pyridin-2-yl | NH—CH(CH₃)₂ | 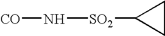 | H |
| Cl | 3,5-F₂-pyridin-2-yl | NH—CHCH₃—CH(CH₃)₂ | CO₂H | H |
| Cl | 3,5-F₂-pyridin-2-yl | NH—CHCH₃—CH(CH₃)₂ | CO—NH—SO₂—CH₃ | H |
| Cl | 3,5-F₂-pyridin-2-yl | NH—CHCH₃—CH(CH₃)₂ | CO—NH—SO₂—C₂H₅ | H |
| Cl | 3,5-F₂-pyridin-2-yl | NH—CHCH₃—CH(CH₃)₂ | 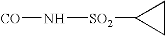 | H |
| Cl | 3,5-F₂-pyridin-2-yl | NH—CH₂—CF₃ | CO₂H | H |
| Cl | 3,5-F₂-pyridin-2-yl | NH—CH₂—CF₃ | CO—NH—SO₂—CH₃ | H |
| Cl | 3,5-F₂-pyridin-2-yl | NH—CH₂—CF₃ | CO—NH—SO₂—C₂H₅ | H |
| Cl | 3,5-F₂-pyridin-2-yl | NH—CH₂—CF₃ | 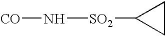 | H |
| Cl | 3,5-F₂-pyridin-2-yl | NH—CH(CH₃)CF₃ | CO₂H | H |
| Cl | 3,5-F₂-pyridin-2-yl | NH—CH(CH₃)CF₃ | CO—NH—SO₂—CH₃ | H |
| Cl | 3,5-F₂-pyridin-2-yl | NH—CH(CH₃)CF₃ | CO—NH—SO₂—C₂H₅ | H |
| Cl | 3,5-F₂-pyridin-2-yl | NH—CH(CH₃)CF₃ | 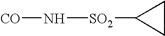 | H |
| Cl | 3,5-F₂-pyridin-2-yl | NH—CH₂—CHF₂ | CO₂H | H |
| Cl | 3,5-F₂-pyridin-2-yl | NH—CH₂—CHF₂ | CO—NH—SO₂—CH₃ | H |
| Cl | 3,5-F₂-pyridin-2-yl | NH—CH₂—CHF₂ | CO—NH—SO₂—C₂H₅ | H |
| Cl | 3,5-F₂-pyridin-2-yl | NH—CH₂—CHF₂ | 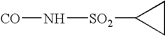 | H |
| Cl | 3,5-Cl₂-pyridin-2-yl | NH—CH(CH₃)₂ | CO—NH—SO₂—C₂H₅ | H |
| Cl | 3,5-Cl₂-pyridin-2-yl | NH—CH(CH₃)₂ | 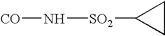 | H |
| Cl | 3,5-Cl₂-pyridin-2-yl | NH—CHCH₃—CH(CH₃)₂ | CO₂H | H |
| Cl | 3,5-Cl₂-pyridin-2-yl | NH—CHCH₃—CH(CH₃)₂ | CO—NH—SO₂—CH₃ | H |
| Cl | 3,5-Cl₂-pyridin-2-yl | NH—CHCH₃—CH(CH₃)₂ | CO—NH—SO₂—C₂H₅ | H |
| Cl | 3,5-Cl₂-pyridin-2-yl | NH—CHCH₃—CH(CH₃)₂ | 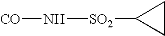 | H |
| Cl | 3,5-Cl₂-pyridin-2-yl | NH—CH₂—CF₃ | CO₂H | H |
| Cl | 3,5-Cl₂-pyridin-2-yl | NH—CH₂—CF₃ | CO—NH—SO₂—CH₃ | H |
| Cl | 3,5-Cl₂-pyridin-2-yl | NH—CH₂—CF₃ | CO—NH—SO₂—C₂H₅ | H |
| Cl | 3,5-Cl₂-pyridin-2-yl | NH—CH₂—CF₃ | 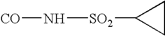 | H |
| Cl | 3,5-Cl₂-pyridin-2-yl | NH—CH(CH₃)CF₃ | CO₂H | H |
| Cl | 3,5-Cl₂-pyridin-2-yl | NH—CH(CH₃)CF₃ | CO—NH—SO₂—CH₃ | H |
| Cl | 3,5-Cl₂-pyridin-2-yl | NH—CH(CH₃)CF₃ | CO—NH—SO₂—C₂H₅ | H |
| Cl | 3,5-Cl₂-pyridin-2-yl | NH—CH(CH₃)CF₃ | 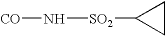 | H |
| Cl | 3,5-Cl₂-pyridin-2-yl | NH—CH₂—CHF₂ | CO₂H | H |
| Cl | 3,5-Cl₂-pyridin-2-yl | NH—CH₂—CHF₂ | CO—NH—SO₂—CH₃ | H |
| Cl | 3,5-Cl₂-pyridin-2-yl | NH—CH₂—CHF₂ | CO—NH—SO₂—C₂H₅ | H |
| Cl | 3,5-Cl₂-pyridin-2-yl | NH—CH₂—CHF₂ | 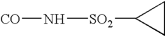 | H |
| Cl | 3,5-F₂-pyridin-4-yl | NH—CH(CH₃)₂ | CO₂H | H |
| Cl | 3,5-F₂-pyridin-4-yl | NH—CH(CH₃)₂ | CO—NH—SO₂—CH₃ | H |
| Cl | 3,5-F₂-pyridin-4-yl | NH—CH(CH₃)₂ | CO—NH—SO₂—C₂H₅ | H |
| Cl | 3,5-F₂-pyridin-4-yl | NH—CH(CH₃)₂ | 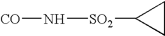 | H |
| Cl | 3,5-F₂-pyridin-4-yl | NH—CHCH₃—CH(CH₃)₂ | CO₂H | H |
| Cl | 3,5-F₂-pyridin-4-yl | NH—CHCH₃—CH(CH₃)₂ | CO—NH—SO₂—CH₃ | H |
| Cl | 3,5-F₂-pyridin-4-yl | NH—CHCH₃—CH(CH₃)₂ | CO—NH—SO₂—C₂H₅ | H |
| Cl | 3,5-F₂-pyridin-4-yl | NH—CHCH₃—CH(CH₃)₂ | 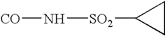 | H |
| Cl | 3,5-F₂-pyridin-4-yl | NH—CH₂—CF₃ | CO₂H | H |
| Cl | 3,5-F₂-pyridin-4-yl | NH—CH₂—CF₃ | CO—NH—SO₂—CH₃ | H |
| Cl | 3,5-F₂-pyridin-4-yl | NH—CH₂—CF₃ | CO—NH—SO₂—C₂H₅ | H |
| Cl | 3,5-F₂-pyridin-4-yl | NH—CH₂—CF₃ | 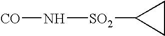 | H |
| Cl | 3,5-F₂-pyridin-4-yl | NH—CH(CH₃)CF₃ | CO₂H | H |
| Cl | 3,5-F₂-pyridin-4-yl | NH—CH(CH₃)CF₃ | CO—NH—SO₂—CH₃ | H |
| Cl | 3,5-F₂-pyridin-4-yl | NH—CH(CH₃)CF₃ | CO—NH—SO₂—C₂H₅ | H |
| Cl | 3,5-F₂-pyridin-4-yl | NH—CH(CH₃)CF₃ | 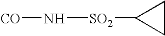 | H |
| Cl | 3,5-F₂-pyridin-4-yl | NH—CH₂—CHF₂ | CO₂H | H |
| Cl | 3,5-F₂-pyridin-4-yl | NH—CH₂—CHF₂ | CO—NH—SO₂—CH₃ | H |
| Cl | 3,5-F₂-pyridin-4-yl | NH—CH₂—CHF₂ | CO—NH—SO₂—C₂H₅ | H |
| Cl | 3,5-F₂-pyridin-4-yl | NH—CH₂—CHF₂ | 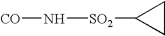 | H |
| Cl | 3,5-Cl₂-pyridin-4-yl | NH—CH(CH₃)₂ | CO—NH—SO₂—C₂H₅ | H |
| Cl | 3,5-Cl₂-pyridin-4-yl | NH—CH(CH₃)₂ | 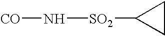 | H |
| Cl | 3,5-Cl₂-pyridin-4-yl | NH—CHCH₃—CH(CH₃)₂ | CO₂H | H |
| Cl | 3,5-Cl₂-pyridin-4-yl | NH—CHCH₃—CH(CH₃)₂ | CO—NH—SO₂—CH₃ | H |
| Cl | 3,5-Cl₂-pyridin-4-yl | NH—CHCH₃—CH(CH₃)₂ | CO—NH—SO₂—C₂H₅ | H |
| Cl | 3,5-Cl₂-pyridin-4-yl | NH—CHCH₃—CH(CH₃)₂ | 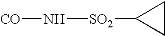 | H |
| Cl | 3,5-Cl₂-pyridin-4-yl | NH—CH₂—CF₃ | CO₂H | H |
| Cl | 3,5-Cl₂-pyridin-4-yl | NH—CH₂—CF₃ | CO—NH—SO₂—CH₃ | H |
| Cl | 3,5-Cl₂-pyridin-4-yl | NH—CH₂—CF₃ | CO—NH—SO₂—C₂H₅ | H |
| Cl | 3,5-Cl₂-pyridin-4-yl | NH—CH₂—CF₃ | 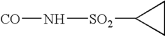 | H |
| Cl | 3,5-Cl₂-pyridin-4-yl | NH—CH(CH₃)CF₃ | CO₂H | H |
| Cl | 3,5-Cl₂-pyridin-4-yl | NH—CH(CH₃)CF₃ | CO—NH—SO₂—CH₃ | H |
| Cl | 3,5-Cl₂-pyridin-4-yl | NH—CH(CH₃)CF₃ | CO—NH—SO₂—C₂H₅ | H |
| Cl | 3,5-Cl₂-pyridin-4-yl | NH—CH(CH₃)CF₃ | 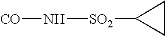 | H |
| Cl | 3,5-Cl₂-pyridin-4-yl | NH—CH₂—CHF₂ | CO₂H | H |
| Cl | 3,5-Cl₂-pyridin-4-yl | NH—CH₂—CHF₂ | CO—NH—SO₂—CH₃ | H |
| Cl | 3,5-Cl₂-pyridin-4-yl | NH—CH₂—CHF₂ | CO—NH—SO₂—C₂H₅ | H |
| Cl | 3,5-Cl₂-pyridin-4-yl | NH—CH₂—CHF₂ | 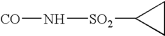 | H |
| Cl | pyridin-2-yl | NH—CH(CH₃)₂ | CO—NH—SO₂—C₂H₅ | H |
| Cl | pyridin-2-yl | NH—CH(CH₃)₂ | 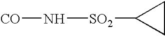 | H |
| Cl | pyridin-2-yl | NH—CHCH₃—CH(CH₃)₂ | CO₂H | H |
| Cl | pyridin-2-yl | NH—CHCH₃—CH(CH₃)₂ | CO—NH—SO₂—CH₃ | H |
| Cl | pyridin-2-yl | NH—CHCH₃—CH(CH₃)₂ | CO—NH—SO₂—C₂H₅ | H |
| Cl | pyridin-2-yl | NH—CHCH₃—CH(CH₃)₂ | 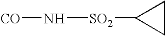 | H |
| Cl | pyridin-2-yl | NH—CH₂—CF₃ | CO₂H | H |
| Cl | pyridin-2-yl | NH—CH₂—CF₃ | CO—NH—SO₂—CH₃ | H |
| Cl | pyridin-2-yl | NH—CH₂—CF₃ | CO—NH—SO₂—C₂H₅ | H |
| Cl | pyridin-2-yl | NH—CH₂—CF₃ | 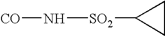 | H |
| Cl | pyridin-2-yl | NH—CH(CH₃)CF₃ | CO₂H | H |
| Cl | pyridin-2-yl | NH—CH(CH₃)CF₃ | CO—NH—SO₂—CH₃ | H |
| Cl | pyridin-2-yl | NH—CH(CH₃)CF₃ | CO—NH—SO₂—C₂H₅ | H |
| Cl | pyridin-2-yl | NH—CH(CH₃)CF₃ | 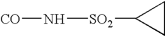 | H |
| Cl | pyridin-2-yl | NH—CH₂—CHF₂ | CO₂H | H |
| Cl | pyridin-2-yl | NH—CH₂—CHF₂ | CO—NH—SO₂—CH₃ | H |
| Cl | pyridin-2-yl | NH—CH₂—CHF₂ | CO—NH—SO₂—C₂H₅ | H |
| Cl | pyridin-2-yl | NH—CH₂—CHF₂ | 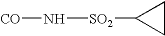 | H |
| Cl | pyridin-4-yl | NH—CH(CH₃)₂ | CO₂H | H |
| Cl | pyridin-4-yl | NH—CH(CH₃)₂ | CO—NH—SO₂—CH₃ | H |
| Cl | pyridin-4-yl | NH—CH(CH₃)₂ | CO—NH—SO₂—C₂H₅ | H |
| Cl | pyridin-4-yl | NH—CH(CH₃)₂ | 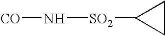 | H |
| Cl | pyridin-4-yl | NH—CHCH₃—CH(CH₃)₂ | CO₂H | H |
| Cl | pyridin-4-yl | NH—CHCH₃—CH(CH₃)₂ | CO—NH—SO₂—CH₃ | H |
| Cl | pyridin-4-yl | NH—CHCH₃—CH(CH₃)₂ | CO—NH—SO₂—C₂H₅ | H |
| Cl | pyridin-4-yl | NH—CHCH₃—CH(CH₃)₂ | 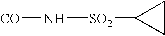 | H |
| Cl | pyridin-4-yl | NH—CH₂—CF₃ | CO₂H | H |
| Cl | pyridin-4-yl | NH—CH₂—CF₃ | CO—NH—SO₂—CH₃ | H |
| Cl | pyridin-4-yl | NH—CH₂—CF₃ | CO—NH—SO₂—C₂H₅ | H |
| Cl | pyridin-4-yl | NH—CH₂—CF₃ | 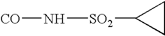 | H |
| Cl | pyridin-4-yl | NH—CH(CH₃)CF₃ | CO₂H | H |
| Cl | pyridin-4-yl | NH—CH(CH₃)CF₃ | CO—NH—SO₂—CH₃ | H |
| Cl | pyridin-4-yl | NH—CH(CH₃)CF₃ | CO—NH—SO₂—C₂H₅ | H |
| Cl | pyridin-4-yl | NH—CH(CH₃)CF₃ | 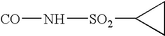 | H |
| Cl | pyridin-4-yl | NH—CH₂—CHF₂ | CO₂H | H |
| Cl | pyridin-4-yl | NH—CH₂—CHF₂ | CO—NH—SO₂—CH₃ | H |
| Cl | pyridin-4-yl | NH—CH₂—CHF₂ | CO—NH—SO₂—C₂H₅ | H |
| Cl | pyridin-4-yl | NH—CH₂—CHF₂ | 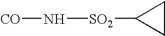 | H |

Compounds of the formula (I) can be prepared, for example, as shown in Scheme 1 from 3-aminopyrazole-4-carboxylic esters of the formula (II) known from the literature (see, for example, U.S. Pat. No. 3,515,715, U.S. Pat. No. 3,634,391 and WO 05/082907) and malonic esters of the formula (III) where R⁸=C₁-C₈-alkyl or aryl:

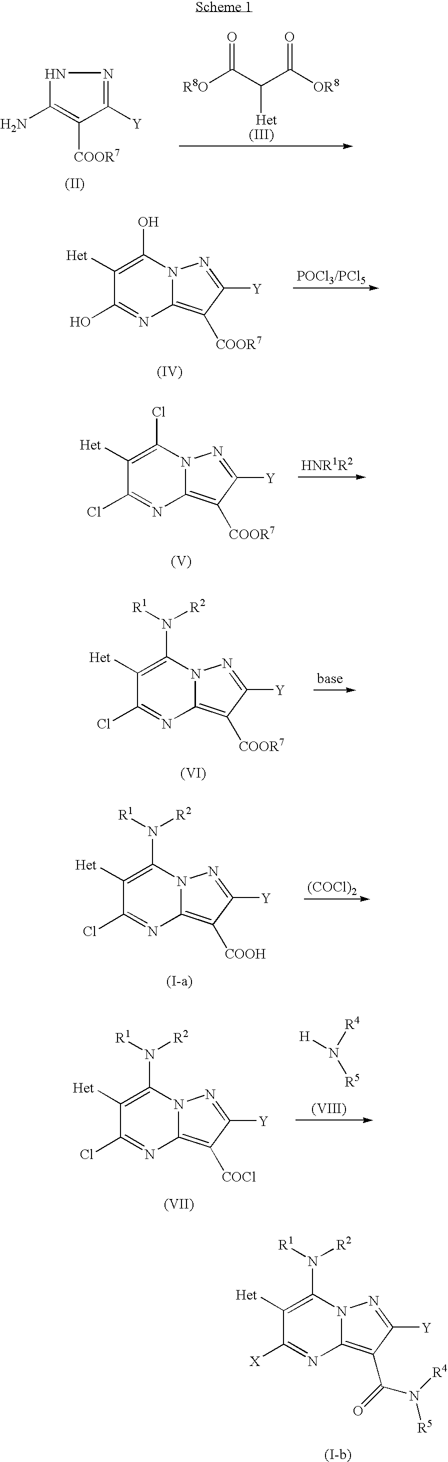

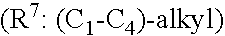

Some of the malonic esters of the formula (III) are known and can be prepared by processes known from the literature (for example WO 04/006913, WO 04/005876).

Certain heterocyclylmalonic esters of the formula

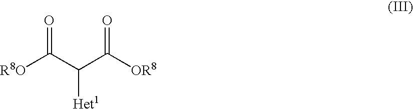

in which
R⁸ has the meaning given above and
Het¹ represents pyrazolyl which is attached in the 1-position and may be mono- or disubstituted by identical or different substituents from the group consisting of haloalkyl having 1 to 7 fluorine and/or chlorine atoms, in particular trifluoromethyl, trichloromethyl, pentafluoroethyl, heptafluoroisopropyl or heptafluoro-n-propyl, or
   represents 4-chloropyrazol-1-yl
      are novel.

The heterocyclylmalonic esters of the formula (III) can be prepared by reacting heterocyclylacetic esters of the formula (IX) in the presence of a base with carbonates of the formula (X).

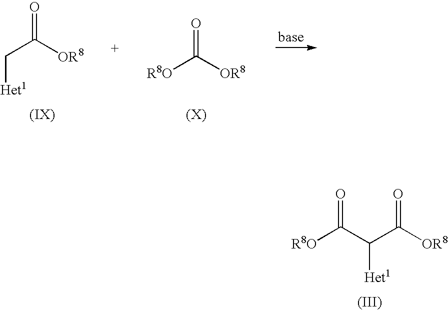

When Het¹ represents pyrazol-1-yl, the heterocyclylacetic esters of the formula (IX) can be prepared, for example by reacting heterocycles of the formula (XI) in the presence of a base with bromoacetic esters, or

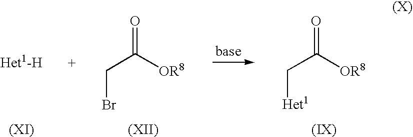

by reacting dicarbonyl compounds of the formula (XIII) with hydrazinoacetic esters of the formula (XIV)

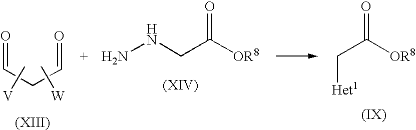

where V and W independently of one another represent hydrogen, haloalkyl having 1 to 7 fluorine and/or chlorine atoms, in particular trifluoromethyl, trichloromethyl, pentafluoroethyl, heptafluoroisopropyl or heptafluoro-n-propyl, and R⁸ in each case has the meaning given above.

The step-wise conversion of the starting materials of the formulae (II) and (III) into amine of the formula (VI) can be carried out, for example, analogously to the process given in WO 04/000 844 and in WO 05/082 907. Hydrolysis of the ester function of compounds of the formula (VI) gives compounds of the formula (I-a). Using these, it is possible to form the acid chlorides of the formula (VII), for example by action of oxalyl chloride.

The compounds of the formula (VIII) used for the further conversion of the acid chlorides of the formula (VII) are known. They are commercially available or can be prepared by known processes familiar to the person skilled in the art as described, for example, in Houben-Weyl, Methoden der Organischen Chemie [Methods of Organic Chemistry].

The compounds of the formula (I) can be obtained by various routes.

Accordingly, the present invention also relates to a process for preparing heterocyclic carboxylic acid derivatives of the general formula (I) as defined above, characterized in that

in process (1)
(a) compounds of the formula (IV)

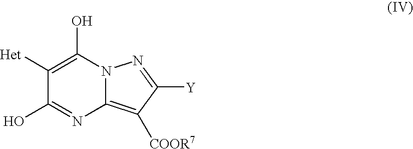

where Het and Y are as defined above and R⁷ represents alkyl are halogenated and
(b) the compounds of the formula (V) obtained in this manner

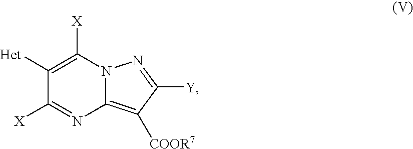

where Het and Y are as defined above, X represents halogen and R⁷ represents alkyl, are reacted with compounds of the formula (XX)

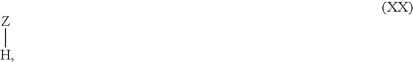

where Z is as defined above, if appropriate in the presence of a base, and
(c) the compounds of the formula (VI) obtained in this manner

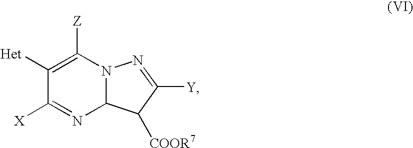

where Het, Y and Z are as defined above, X represents halogen and R⁷ represents alkyl, are hydrolysed in the presence of acid or base, and
(d) the compounds of the formula (I-a) obtained in this manner

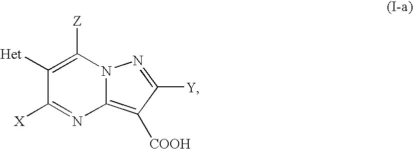

where Het, Y and Z are as defined above and X represents halogen, are reacted with compounds of the formula (VIII)

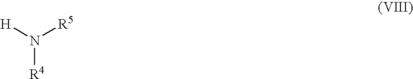

where R⁴ and R⁵ are as defined above. Methods for forming amide bonds are known from the literature (for example METHODS OF ORGANIC CHEMISTRY (Houben Weyl): ‘Synthesis of Peptides and Peptidomimetics’, Volume E22a, Goodman et al. (Eds.), Georg Thieme Verlag Stuttgart, 2002).

The compounds of the formula (I) where Het, X, Y, Z and R⁵ are as defined above and R⁴ represents hydrogen can be reacted with bases containing M¹ as cation to give compounds of the formula (I) where Het, X, Y, Z, R⁵ and M¹ are as defined above and R³ represents

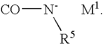

The present invention also relates to a process for preparing compounds of the general formula (IV) as defined above, characterized in that

in process (2)
(e) compounds of the formula (II)

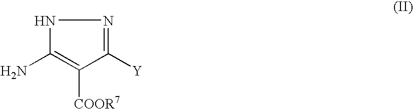

where Y is as defined above and R⁷ represents alkyl
are reacted with malonic esters of the formula (III)

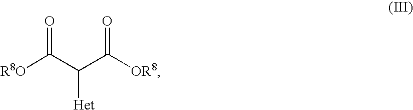

where Het is as defined above and R⁸ represents alkyl or aryl;
or that in process (3)
(f) compounds of the formula (XXI)

Het²-Hal (XXI)

where Het² represents six-membered heterocycles and Hal represents bromine or iodine, are reacted with malononitrile of the formula (XVII)

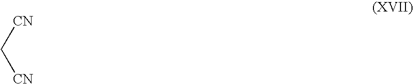

preferably with action of a base and in the presence of catalytically active amounts of a transition metal of transition group VIII of the Periodic Table of the Elements (group 10 according to IUPAC), for example nickel, palladium or platinum, in particular in the presence of a palladium catalyst (suitable palladium catalysts contain ligands, such as, for example, phosphines), and
(g) the compounds of the formula (XVIII) obtained in this manner

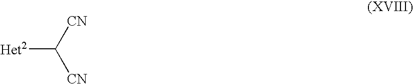

where Het² represents six-membered heterocycles, are hydrolysed in the presence of acid or base and
(h) the compounds of the formula (XIX) obtained in this manner

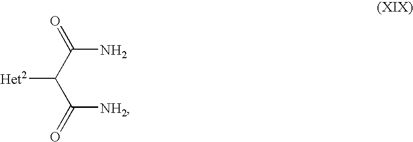

where Het² represents six-membered heterocycles,
are reacted with compounds of the formula (II)

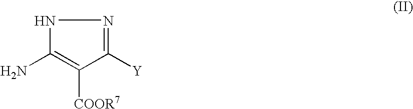

where Y is as defined above and R⁷ represents alkyl, to give compounds of the formula (IV-a)

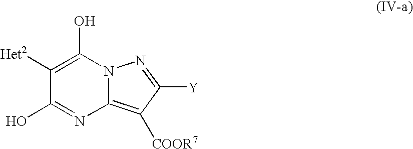

where Y is as defined above, Het² represents six-membered heterocycles and R⁷ represents alkyl.

Compounds of the formula (VI) in which X represents fluorine or iodine are obtained from the corresponding compounds of the formula (VI) in which X represents chlorine or bromine by reaction with the appropriate alkali metal or alkaline earth metal halides.

Compounds of the formula (VI) in which X represents cyano are obtained from the corresponding compounds of the formula (VI) as defined above by reaction with the appropriate alkali metal or alkaline earth metal cyanides.

R¹ represents alkyl, preferably C₁-C₁-alkyl. R⁸ represents alkyl, preferably C₁-C₈-alkyl, or represents aryl. X represents halogen, preferably chlorine or bromine.

The substituents Het, Het², X, Y, Z, R¹, R², R⁴ and R⁵ referred to in the formulae (I-a), (I-b), (II), (III), (IV), (IV-a), (V), (VI), (VII), (XIII), (XXI), (XVII), (XVIII) and (XIX) preferably have those meanings already mentioned as being preferred for these radicals in connection with the description of the compounds of the formula (I) according to the invention.

The compounds of the formulae (VIII), (XX), (XXI) and (XVII) are known. They are commercially available or can be prepared by known processes familiar to the person skilled in the art, as described, for example, in Houben-Weyl, Methoden der Organischen Chemie. The compounds of the formula (II) are known from the literature (see, for example, U.S. Pat. No. 3,515,715, U.S. Pat. No. 3,634,391 and WO 05/082907).

The processes according to the invention for preparing the compounds of the formula (I) are preferably carried out using one or more reaction auxiliaries.

Suitable reaction auxiliaries are, if appropriate, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN), or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroine, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and n-methylpyrrolidone, and also dimethyl sulphoxide, tetramethylene sulphone and hexamethylphosphoric triamide.

The reaction temperatures in the processes according to the invention can be varied within a relatively wide range. In general, the reactions are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use a relatively large excess of one of the components used in each case. Work-up in the processes according to the invention is in each case carried out by customary methods (cf. the Preparation Examples).

In the literature it has already been described how the action of various active compounds can be boosted by addition of ammonium salts. The salts in question, however, are detersive salts (for example WO 95/017817) or salts which have relatively long alkyl substituents and/or aryl substituents and which have a permeabilizing action or which increase the active compounds' solubility (for example EP-A 0 453 086, EP-A 0 664 081, FR-A 2 600 494, U.S. Pat. No. 4,844,734, U.S. Pat. No. 5,462,912, U.S. Pat. No. 5,538,937, US-A 03/0224939, US-A 05/0009880, US-A 05/0096386). Moreover, the prior art describes the action only for particular active compounds and/or particular applications of the corresponding compositions. In other cases, they are salts of sulphonic acids where the acids for their part have paralysing action on the insects (U.S. Pat. No. 2,842,476). A boost of action by ammonium sulphate, for example, is described by way of example for the herbicides glyphosate and phosphinothricin (U.S. Pat. No. 6,645,914, EP-A2 0 036 106). However, this prior art neither discloses nor suggests a corresponding action for insecticides.

The use of ammonium sulphate as formulating assistant has also been described for certain active compounds and applications (WO 92/16108), but its purpose therein is to stabilize the formulation, not to boost the action.

It has now been found, entirely surprisingly, that the action of insecticides and/or acaricides from the class of the heterocyclic carboxylic acid derivatives (I) can be boosted significantly through the addition of ammonium salts or phosphonium salts to the application solution or through the incorporation of these salts into a formulation comprising heterocyclic carboxylic acid derivatives (I). The present invention therefore provides for the use of ammonium salts or phosphonium salts for boosting the action of crop protection compositions which comprise as their active compound insecticidal and/or acaricidal heterocyclic carboxylic acid derivatives (I). The invention likewise provides compositions which comprise insecticidal and/or acaricidal heterocyclic carboxylic acid derivatives (I) and action-boosting ammonium salts or phosphonium salts, including not only formulated active compounds but also ready-to-use compositions (spray liquors). The invention further provides, finally, for the use of these compositions for controlling harmful insects and/or spider mites.

Ammonium salts and phosphonium salts which inventively boost the activity of crop protection compositions comprising heterocyclic carboxylic acid derivatives (I) are defined by formula (XV)

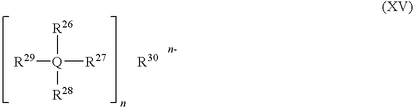

in which
Q represents nitrogen or phosphorus,
Q preferably represents nitrogen,
R²⁶, R²⁷, R²⁸ and R²⁹ independently of one another represent hydrogen or in each case optionally substituted C₁-C₈-alkyl or mono- or polyunsaturated, optionally substituted C₁-C₈-alkylene, the substituents being selectable from halogen, nitro and cyano,
R²⁶, R²⁷, R²⁸ and R²⁹ independently of one another preferably represent hydrogen or in each case optionally substituted C₁-C₄-alkyl, the substituents being selectable from halogen, nitro and cyano,
R²⁶, R²⁷, R²⁸ and R²⁹ independently of one another particularly preferably represent hydrogen, methyl, ethyl, n-propyl, isopropyl, n-butyl, isobutyl, sec-butyl or tert-butyl,
R²⁶, R²⁷, R²⁸ and R²⁹ very particularly preferably represent hydrogen,
R²⁶, R²⁷, R²⁸ and R²⁹ furthermore very particularly preferably all represent methyl or all represent ethyl,
n represents 1, 2, 3 or 4,
n preferably represents 1 or 2,
R³⁰ represents an organic or inorganic anion,
R³⁰ preferably represents hydrogencarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate, citrate or oxalate,
R³⁰ furthermore preferably represents carbonate, pentaborate, sulphite, benzoate, hydrogenoxalate, hydrogencitrate, methylsulphate or tetrafluoroborate,
R³⁰ particularly preferably represents lactate, sulphate, nitrate, thiosulphate, thiocyanate, citrate, oxalate or formate,
R³⁰ moreover particularly preferably represents monohydrogenphosphate or dihydrogenphosphate and
R³⁰ very particularly preferably represents sulphate, thiocyanate, dihydrogenphosphate or monohydrogenphosphate.

The ammonium salts and phosphonium salts of the formula (XV) can be used in a broad concentration range to boost the activity of crop protection compositions comprising heterocyclic carboxylic acid derivatives (I). In general the ammonium salts or phosphonium salts are used in the ready-to-use crop protection composition in a concentration of 0.5 to 80 mmol/l, preferably 0.75 to 37.5 mmol/l, more preferably 1.5 to 25 mmol/l. In the case of a formulated product the ammonium salt and/or phosphonium salt concentration in the formulation is chosen such that it is within these stated general, preferred or particularly preferred ranges after the formulation has been diluted to the desired active-ingredient concentration. The concentration of the salt in the formulation is typically 1%-50% by weight. The concentration of the active ingredient in the formulation is typically 0.01%-50% by weight.

In one preferred embodiment of the invention the activity is boosted by adding to the crop protection compositions not only an ammonium salt and/or phosphonium salt but also, additionally, a penetrant. It is considered entirely surprising that even in these cases an even greater boost to activity is observed. The present invention therefore likewise provides for the use of a combination of penetrant and ammonium salts and/or phosphonium salts to boost the activity of crop protection compositions which comprise insecticidal and/or acaricidal heterocyclic carboxylic acid derivatives (I) as active compound. The invention likewise provides compositions which comprise insecticidal and/or acaricidal heterocyclic carboxylic acid derivatives (I), penetrants and ammonium salts and/or phosphonium salts, including specifically not only formulated active compounds but also ready-to-use compositions (spray liquors). The invention additionally provides, finally, for the use of these compositions for controlling harmful insects.

Suitable penetrants in the present context include all those substances which are typically used to enhance the penetration of active agrochemical compounds into plants. Penetrants are defined in this context by their ability to penetrate from the aqueous spray liquor and/or from the spray coating into the cuticle of the plant and thereby to increase the mobility of active compounds in the cuticle. The method described in the literature (Baur et al., 1997, Pesticide Science 51, 131-152) can be used in order to determine this property.

Examples of suitable penetrants include alkanol alkoxylates. Penetrants of the invention are alkanol alkoxylates of the formula

R—O-(-AO)ᵥ-R′ (XVI)

in which
R represents straight-chain or branched alkyl having 4 to 20 carbon atoms,
R′ represents hydrogen, methyl, ethyl, n-propyl, isopropyl, n-butyl, isobutyl, tert-butyl, n-pentyl or n-hexyl,
AO represents an ethylene oxide radical, a propylene oxide radical, a butylene oxide radical or is mixtures of ethylene oxide and propylene oxide radicals or butylene oxide radicals, and
v represents a number from 2 to 30.

One preferred group of penetrants are alkanol alkoxylates of the formula

R—O-(-EO-)ₙ-R′ (XVI-a)

in which
R is as defined above,
R′ is as defined above,
EO represents —CH₂—CH₂—O—, and
n represents a number from 2 to 20.

A further preferred group of penetrants are alkanol alkoxylates of the formula

R—O-(-EO-)ₚ-(-PO—)_{q}—R′ (XVI-b)

in which
R is as defined above,
R′ is as defined above,
EO represents —CH₂—CH₂—O—,
PO represents

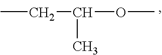

p represents a number from 1 to 10, and
q represents a number from 1 to 10.

A further preferred group of penetrants are alkanol alkoxylates of the formula

R—O—(—PO-)ᵣ-(EO-)ₛ-R′ (XVI-c)

in which
R is as defined above,
R′ is as defined above,
EO represents —CH₂—CH₂—O—,
PO represents

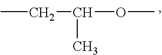

r represents a number from 1 to 10, and
s represents a number from 1 to 10.

A further preferred group of penetrants are alkanol alkoxylates of the formula

R—O-(-EO-)ₚ-(-BO—)_{q}—R′ (XVI-d)

in which
R and R′ are as defined above,
EO represents CH₂—CH₂—O—,
BO represents

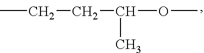

p represents a number from 1 to 10 and
q represents a number from 1 to 10.

A further preferred group of penetrants are alkanol alkoxylates of the formula

R—O—(—BO-)ᵣ-(-EO-)_{S}-R′ (XVI-e)

in which
R and R′ are as defined above,
BO represents

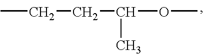

EO represents CH₂—CH₂—O—,
r represents a number from 1 to 10 and
s represents a number from 1 to 10.

A further preferred group of penetrants are alkanol alkoxylates of the formula

CH₃—(CH₂)ₜ—CH₂—O—(—CH₂—CH₂—O—)ᵤ—R′ (XVI-f)

in which
R¹ is as defined above,
t represents a number from 8 to 13,
u represents a number from 6 to 17.

In the formulae indicated above,
R preferably represents butyl, isobutyl, n-pentyl, isopentyl, neopentyl, n-hexyl, isohexyl, n-octyl, isooctyl, 2-ethylhexyl, nonyl, isononyl, decyl, n-dodecyl, isododecyl, lauryl, myristyl, isotridecyl, trimethylnonyl, palmityl, stearyl or eicosyl.

As an example of an alkanol alkoxylate of the formula (XVI-c) mention may be made of 2-ethylhexyl alkoxylate of the formula

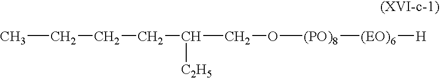

in which
EO represents —CH₂—CH₂—O—,
PO represents

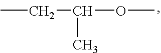

the numbers 8 and 6 represent average values.

As an example of an alkanol alkoxylate of the formula (XVI-d) mention may be made of the formula

CH₃—(CH₂)₁₀—O-(-EO-)₆-(-BO—)₂—CH₃ (XVI-d-1)

in which
EO represents CH₂—CH₂—O—,
BO represents

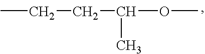

and
the numbers 10, 6 and 2 represent average values.

Particularly preferred alkanol alkoxylates of the formula (XVI-f) are compounds of this formula in which
t represents a number from 9 to 12 and
u represents a number from 7 to 9.

Mention may be made with very particular preference of alkanol alkoxylate of the formula (XVI-f-1)

CH₃—(CH₂)ₜ—CH₂—O—(—CH₂—CH₂—O—)ᵤ—H (XVI-f-1)

in which
t stands for the average value 10.5 and
u stands for the average value 8.4.

A general definition of the alkanol alkoxylates is given by the formulae above. These substances are mixtures of compounds of the stated type with different chain lengths. The indices therefore have average values which may also deviate from whole numbers.

The alkanol alkoxylates of the formulae stated are known and in some cases are available commercially or can be prepared by known methods (cf. WO 98/35 553, WO 00/35 278 and EP-A 0 681 865).

Suitable penetrants also include, for example, substances which promote the availability of the compounds of the formula (I) in the spray coating. These include, for example, mineral or vegetable oils. Suitable oils are all mineral or vegetable oils—modified or otherwise—which can typically be used in agrochemical compositions. Mention may be made by way of example of sunflower oil, rapeseed oil, olive oil, castor oil, colza oil, maize seed oil, cotton seed oil and soybean oil, or the esters of said oils. Preference is given to rapeseed oil, sunflower oil and their methyl or ethyl esters.

The concentration of penetrant in the compositions of the invention can be varied within a wide range. In the case of a formulated crop protection composition it is in general 1% to 95%, preferably 1% to 55%, more preferably 15%-40% by weight. In the ready-to-use compositions (spray liquors) the concentrations are generally between 0.1 and 10 g/l, preferably between 0.5 and 5 g/l.

Inventively emphasized combinations of active compound, salt and penetrant are listed in the table below. Here, “as per test” means that any compound that acts as a penetrant in the cuticle penetration test (Baur et al., 1997, Pesticide Science 51, 131-152) is suitable.

| | Active | | |
|---|---|---|---|
| # | compound | Salt | Penetrant |
| 1 | I-16 | ammonium sulphate | as per test |
| 2 | I-16 | ammonium lactate | as per test |
| 3 | I-16 | ammonium nitrate | as per test |
| 4 | I-16 | ammonium thiosulphate | as per test |
| 5 | I-16 | ammonium thiocyanate | as per test |
| 6 | I-16 | ammonium citrate | as per test |
| 7 | I-16 | ammonium oxalate | as per test |
| 8 | I-16 | ammonium formate | as per test |
| 9 | I-16 | ammonium hydrogenphosphate | as per test |
| 10 | I-16 | ammonium dihydrogenphosphate | as per test |
| 11 | I-16 | ammonium carbonate | as per test |
| 12 | I-16 | ammonium benzoate | as per test |
| 13 | I-16 | ammonium sulphite | as per test |
| 14 | I-16 | ammonium hydrogenoxalate | as per test |
| 15 | I-16 | ammonium hydrogencitrate | as per test |
| 16 | I-16 | ammonium acetate | as per test |
| 17 | I-16 | tetramethylammonium sulphate | as per test |
| 18 | I-16 | tetramethylammonium lactate | as per test |
| 19 | I-16 | tetramethylammonium nitrate | as per test |
| 20 | I-16 | tetramethylammonium thiosulphate | as per test |
| 21 | I-16 | tetramethylammonium thiocyanate | as per test |
| 22 | I-16 | tetramethylammonium citrate | as per test |
| 23 | I-16 | tetramethylammonium oxalate | as per test |
| 24 | I-16 | tetramethylammonium formate | as per test |
| 25 | I-16 | tetramethylammonium hydrogenphosphate | as per test |
| 26 | I-16 | tetramethylammonium dihydrogenphosphate | as per test |
| 27 | I-16 | tetraethylammonium sulphate | as per test |
| 28 | I-16 | tetraethylammonium lactate | as per test |
| 29 | I-16 | tetraethylammonium nitrate | as per test |
| 30 | I-16 | tetraethylammonium thiosulphate | as per test |
| 31 | I-16 | tetraethylammonium thiocyanate | as per test |
| 32 | I-16 | tetraethylammonium citrate | as per test |
| 33 | I-16 | tetraethylammonium oxalate | as per test |
| 34 | I-16 | tetraethylammonium formate | as per test |
| 35 | I-16 | tetraethylammonium hydrogenphosphate | as per test |
| 36 | I-16 | tetraethylammonium dihydrogenphosphate | as per test |

Crop protection compositions of the invention may also comprise further components, examples being surfactants and/or dispersing assistants or emulsifiers.

Suitable nonionic surfactants and/or dispersing assistants include all substances of this type that can typically be used in agrochemical compositions. Preferably mention may be made of polyethylene oxide-polypropylene oxide block copolymers, polyethylene glycol ethers of linear alcohols, reaction products of fatty acids with ethylene oxide and/or propylene oxide, and also polyvinyl alcohol, polyvinylpyrrolidone, copolymers of polyvinyl alcohol and polyvinylpyrrolidone, and copolymers of (meth)acrylic acid and (meth)acrylic esters, and additionally alkyl ethoxylates and alkylaryl ethoxylates, which optionally may be phosphated and optionally may be neutralized with bases, mention being made, by way of example, of sorbitol ethoxylates, and, as well, polyoxyalkylenamine derivatives.

Suitable anionic surfactants include all substances of this type that can typically be used in agrochemical compositions. Preference is given to alkali metal salts and alkaline earth metal salts of alkylsulphonic acids or alkylarylsulphonic acids.

A further preferred group of anionic surfactants and/or dispersing assistants are the following salts that are of low solubility in plant oil: salts of polystyrenesulphonic acids, salts of polyvinylsulphonic acids, salts of naphthalenesulphonic acid-formaldehyde condensation products, salts of condensation products of naphthalenesulphonic acid, phenolsulphonic acid and formaldehyde, and salts of lignosulphonic acid.

Suitable additives which may be included in the formulations of the invention are emulsifiers, foam inhibitors, preservatives, antioxidants, colorants and inert filling materials.

Preferred emulsifiers are ethoxylated nonylphenols, reaction products of alkylphenols with ethylene oxide and/or propylene oxide, ethoxylated arylalkylphenols, and also ethoxylated and propoxylated arylalkylphenols, and also sulphated or phosphated arylalkyl ethoxylates and/or arylalkyl ethoxypropoxylates, mention being made by way of example of sorbitan derivatives, such as polyethylene oxide-sorbitan fatty acid esters, and sorbitan fatty acid esters.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

The compounds of the formula (I) according to the invention are distinguished in particular by strong activity against insects, parasites from the subclass of the Acari (Acarina) (such as mites, spider mites and/or ticks) and/or nematodes.

The compounds according to the invention have strong fungicidal activity and can be used for controlling phytopathogenic harmful fungi in crop protection and in the protection of materials.

Fungicides can be used in crop protection for controlling Plasmodiophoromycetes, Oomycetes, Chytridiomycetes, Zygomycetes, Ascomycetes, Basidiomycetes and Deuteromycetes.

Some pathogens causing fungal diseases which come under the generic names listed above may be mentioned as examples, but not by way of limitation:

diseases caused by powdery mildew pathogens, such as, for example,

Blumeria species, such as, for example, Blumeria graminis;

Podosphaera species, such as, for example, Podosphaera leucotricha;

Sphaerotheca species, such as, for example, Sphaerotheca fuliginea;

Uncinula species, such as, for example, Uncinula necator;

diseases caused by rust disease pathogens, such as, for example,

Gymnosporangium species, such as, for example, Gymnosporangium sabinae;

Hemileia species, such as, for example, Hemileia vastatrix;

Phakopsora species, such as, for example, Phakopsora pachyrhizi and Phakopsora meibomiae;

Puccinia species, such as, for example, Puccinia recondita;

Uromyces species, such as, for example, Uromyces appendiculatus;

diseases caused by pathogens from the group of the Oomycetes, such as, for example,

Bremia species, such as, for example, Bremia lactucae;

Peronospora species, such as, for example, Peronospora pisi or P. brassicae;

Phytophthora species, such as, for example Phytophthora infestans;

Plasmopara species, such as, for example, Plasmopara viticola;

Pseudoperonospora species, such as, for example, Pseudoperonospora humuli or Pseudoperonospora cubensis;

Pythium species, such as, for example, Pythium ultimum;

leaf blotch diseases and leaf wilt diseases caused, for example, by

Alternaria species, such as, for example, Alternaria solani;

Cercospora species, such as, for example, Cercospora beticola;

Cladiosporium species, such as, for example, Cladiosporium cucumerinum;

Cochliobolus species, such as, for example, Cochliobolus sativus

(conidia form: Drechslera, Syn: Helminthosporium);

Colletotrichum species, such as, for example, Colletotrichum lindemuthanium;

Cycloconium species, such as, for example, Cycloconium oleaginum;

Diaporthe species, such as, for example, Diaporthe citri;

Elsinoe species, such as, for example, Elsinoe fawcettii;

Gloeosporium species, such as, for example, Gloeosporium laeticolor;

Glomerella species, such as, for example, Glomerella cingulata;

Guignardia species, such as, for example, Guignardia bidwelli;

Leptosphaeria species, such as, for example, Leptosphaeria maculans;

Magnaporthe species, such as, for example, Magnaporthe grisea;

Mycosphaerella species, such as, for example, Mycosphaerella graminicola;

Phaeosphaeria species, such as, for example, Phaeosphaeria nodorum;

Pyrenophora species, such as, for example, Pyrenophora teres;

Ramularia species, such as, for example, Ramularia collo-cygni;

Rhynchosporium species, such as, for example, Rhynchosporium secalis;

Septoria species, such as, for example, Septoria apii;

Typhula species, such as, for example, Typhula incarnata;

Venturia species, such as, for example, Venturia inaequalis;

root and stem diseases caused, for example, by

Corticium species, such as, for example, Corticium graminearum;

Fusarium species, such as, for example, Fusarium oxysporum;

Gaeumannomyces species, such as, for example, Gaeumannomyces graminis;

Rhizoctonia species, such as, for example, Rhizoctonia solani;

Tapesia species, such as, for example, Tapesia acuformis;

Thielaviopsis species, such as, for example, Thielaviopsis basicola;

ear and panicle diseases (including maize cobs) caused, for example, by

Alternaria species, such as, for example, Alternaria spp.;

Aspergillus species, such as, for example, Aspergillus flavus;

Cladosporium species, such as, for example, Cladosporium spp.;

Claviceps species, such as, for example, Claviceps purpurea;

Fusarium species, such as, for example, Fusarium culmorum;

Gibberella species, such as, for example, Gibberella zeae;

Monographella species, such as, for example, Monographella nivalis;

diseases caused by smut fungi, such as, for example,

Sphacelotheca species, such as, for example, Sphacelotheca reiliana;

Tilletia species, such as, for example, Tilletia caries;

Urocystis species, such as, for example, Urocystis occulta;

Ustilago species, such as, for example, Ustilago nuda;

fruit rot caused, for example, by

Aspergillus species, such as, for example, Aspergillus flavus;

Botrytis species, such as, for example, Botrytis cinerea;

Penicillium species, such as, for example, Penicillium expansum;

Sclerotinia species, such as, for example, Sclerotinia sclerotiorum;

Verticilium species, such as, for example, Verticilium alboatrum;

seed- and soil-borne rot and wilt diseases, and also diseases of seedlings, caused, for example, by

Fusarium species, such as, for example, Fusarium culmorum;

Phytophthora species, such as, for example, Phytophthora cactorum;

Pythium species, such as, for example, Pythium ultimum;

Rhizoctonia species, such as, for example, Rhizoctonia solani;

Sclerotium species, such as, for example, Sclerotium rolfsii;

cancerous diseases, galls and witch's broom caused, for example, by

Nectria species, such as, for example, Nectria galligena;

wilt diseases caused, for example, by

Monilinia species, such as, for example, Monilinia laxa;

deformations of leaves, flowers and fruits caused, for example, by

Taphrina species, such as, for example, Taphrina deformans;

degenerative diseases of woody plants caused, for example, by

Esca species, such as, for example, Phaemoniella clamydospora, Phaeoacremonium aleophilum and Formitiporia mediterranea;

diseases of flowers and seeds caused, for example, by

Botrytis species, such as, for example, Botrytis cinerea;

diseases of plant tubers caused, for example, by

Rhizoctonia species, such as, for example, Rhizoctonia solani;

Helminthosporium species, such as, for example, Helminthosporium solani;

diseases cause by bacterial pathogens, such as, for example,

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora.

With preference, it is possible to control the following diseases of soya beans:

fungal diseases on leaves, stems, pods and seeds, caused, for example, by

alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola)

Fungal diseases on roots and the stem base, caused, for example, by

black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also have a potent strengthening effect in plants. They are therefore suitable for mobilizing the plants' defences against attack by unwanted phytopathogenic harmful fungi.

Plant-strengthening (resistance-inducing) substances are understood as meaning, in the present context, those substances which are capable of stimulating the defence system of plants in such a way that, when subsequently inoculated with unwanted phytopathogenic harmful fungi, the treated plants display a substantial degree of resistance to these phytopathogenic harmful fungi.

Thus, the substances according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compound combinations are well tolerated by plants at the concentrations required for controlling plant diseases permits a treatment of above-ground parts of plants, of propagation stock and seed, and of the soil.

Here, the compounds according to the invention can be used with particularly good results for controlling cereal diseases, such as, for example, against Puccinia species, and diseases in viticulture and the cultivation of fruit and vegetables, such as, for example, against Botrytis, Venturia- or Alternaria species.

The compounds according to the invention are also suitable for increasing the harvest yield. Moreover, they have reduced toxicity and are highly compatible with plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable solid carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances, herbicides, safeners, fertilizers or semiochemicals.

A mixture with other known active compounds, such as herbicides, fertilizers, growth regulators, safeners, semiochemicals, or else with agents for improving the plant properties, is also possible.

When used as insecticides or fungicides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

When used as insecticides or fungicides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compounds are employed in a customary manner appropriate for the use forms.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, habitat or storage space by the customary treatment methods, for example by immersion, watering, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particular advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco, rice, canola and oilseed rape. Traits that are emphasized are in particular increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The control of animal pests and/or phytopathogenic harmful fungi by treating the seeds of plants has been known for a long time and is subject-matter of continuous improvements. However, the treatment of seed frequently entails a series of problems which cannot always be solved in a satisfactory manner. Thus, it is desirable to develop methods for protecting the seed and the germinating plant which dispense with the additional application of crop protection agents after sowing or after the emergence of the plants or where additional applications are at least reduced. It is furthermore desirable to optimize the amount of active compound employed in such a way as to provide maximum protection for the seed and the germinating plant from attack by animal pests and/or phytopathogenic harmful fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore in particular also relates to a method for the protection of seed and germinating plants from attack by animal pests and/or phytopathogenic harmful fungi, by treating the seed with a composition according to the invention.

The invention likewise relates to the use of the compositions according to the invention for the treatment of seed for protecting the seed and the germinating plant from animal pests and/or phytopathogenic harmful fungi.

Furthermore, the invention relates to seed which has been treated with a composition according to the invention so as to afford protection from animal pests and/or phytopathogenic harmful fungi.

One of the advantages of the present invention is that the particular systemic properties of the compositions according to the invention mean that treatment of the seed with these compositions not only protects the seed itself, but also the resulting plants after emergence, from animal pests and/or phytopathogenic harmful fungi. In this manner, the immediate treatment of the crop at the time of sowing or shortly thereafter can be dispensed with.

Furthermore, it must be considered as advantageous that the mixtures according to the invention can also be employed in particular in transgenic seed.

The compositions according to the invention are suitable for protecting seed of any plant variety which is employed in agriculture, in the greenhouse, in forests or in horticulture. In particular, this takes the form of seed of cereals (such as wheat, barley, rye, millet and oats), maize, cotton, soya beans, rice, potatoes, sunflowers, beans, coffee, beet (for example sugar beet and fodder beet), peanuts, vegetables (such as tomatoes, cucumbers, onions and lettuce), lawns and ornamental plants. The treatment of seed of cereals (such as wheat, barley, rye and oats), maize and rice is of particular importance.

In the context of the present invention, the composition according to the invention is applied to the seed either alone or in a suitable formulation. Preferably, the seed is treated in a state which is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, for example, it is possible to use seed which has been harvested, cleaned and dried to a moisture content of below 15% by weight. Alternatively, it is also possible to use seed which, after drying, has, for example, been treated with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the composition to the seed in the form of a suitable formulation. Suitable formulations and methods for the treatment of seed are known to the skilled worker and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations which can be used according to the invention can be converted into customary seed dressing formulations, such as solutions, emulsions, suspensions, powders, foams, slurries or other coating materials for seed, and also ULV formulations.

These formulations are prepared in a known manner by mixing the active compounds or active compound combinations with customary additives, such as, for example, customary extenders and also solvents or diluents, colorants, wetting agents, dispersants, emulsifiers, defoamers, preservatives, secondary thickeners, adhesives, gibberellins and water as well.

Suitable colorants that may be present in the seed dressing formulations which can be used according to the invention include all colorants customary for such purposes. Use may be made both of pigments, of sparing solubility in water, and of colorants, which are soluble in water. Examples that may be mentioned include the colorants known under the designations rhodamine B, C.I. Pigment Red 112, and C.I. Solvent Red 1.

Suitable wetting agents that may be present in the seed dressing formulations which can be used according to the invention include all substances which promote wetting and are customary in the formulation of active agrochemical substances. With preference it is possible to use alkylnaphthalene-sulphonates, such as diisopropyl- or diisobutylnaphthalene-sulphonates.

Suitable dispersants and/or emulsifiers that may be present in the seed dressing formulations which can be used according to the invention include all nonionic, anionic, and cationic dispersants which are customary in the formulation of active agrochemical substances. With preference, it is possible to use nonionic or anionic dispersants or mixtures of nonionic or anionic dispersants. Particularly suitable nonionic dispersants are ethylene oxide-propylene oxide block polymers, alkylphenol polyglycol ethers, and tristyrylphenol polyglycol ethers, and their phosphated or sulphated derivatives. Particularly suitable anionic dispersants are lignosulphonates, polyacrylic salts, and arylsulphonate-formaldehyde condensates.

Suitable defoamers that may be present in the seed dressing formulations which can be used according to the invention include all foam-inhibiting substances which are customary in the formulation of active agrochemical substances. With preference it is possible to use silicone defoamers and magnesium stearate.

Suitable preservatives that may be present in the seed dressing formulations which can be used according to the invention include all substances which can be used for such purposes in agrochemical compositions. By way of example, mention may be made of dichlorophen and benzyl alcohol hemiformal.

Suitable secondary thickeners that may be present in the seed dressing formulations which can be used according to the invention include all substances which can be used for such purposes in agrochemical compositions. Preferred suitability is possessed by cellulose derivatives, acrylic acid derivatives, xanthan, modified clays, and highly disperse silica.

Suitable adhesives that may be present in the seed dressing formulations which can be used according to the invention include all customary binders which can be used in seed dressing. With preference, mention may be made of polyvinylpyrrolidone, polyvinyl acetate, polyvinyl alcohol and tylose.

Suitable gibberellins that may be present in the seed dressing formulations which can be used according to the invention are preferably the gibberellins A1, A3 (=gibberellinic acid), A4 and A7; particularly preferably, gibberellinic acid is used. The gibberellins are known (cf. R. Wegler “Chemie der Pflanzenschutz- and Schädlingsbekämpfungsmittel”, Vol. 2, Springer Verlag, 1970, pp. 401-412).

The seed dressing formulations which can be used according to the invention may be used directly or after dilution with water beforehand to treat seed of any of a very wide variety of types. For instance, the concentrates or the preparations obtainable therefrom by dilution with water may be used to dress the seed of cereals, such as wheat, barley, rye, oats, and triticale, and also the seed of maize, rice, oilseed rape, peas, field beans, cotton, sunflowers, and beets, or else vegetable seed of any of a very wide variety of kinds. The seed dressing formulations which can be used according to the invention or their dilute preparations may also be used to dress seed of transgenic plants. In this context, synergistic effects may also arise in interaction with the substances formed by expression.

Suitable mixing equipment for treating seed with the seed dressing formulations which can be used according to the invention or the preparations prepared from them by adding water includes all mixing equipment which can commonly be used for dressing. The specific procedure adopted when dressing comprises introducing the seed into a mixer, adding the particular desired amount of seed dressing formulation, either as it is or following dilution with water beforehand, and carrying out mixing until the formulation is uniformly distributed on the seed. Optionally, a drying operation follows.

The application rate of the seed dressing formulations which can be used according to the invention may be varied within a relatively wide range. It depends on the respective content of the active compounds in the formulations and on the seed. In general, the application rates of active compound combination are between 0.001 and 50 g per kilogram of seed, preferably between 0.01 and 15 g per kilogram of seed.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reductions in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100- to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, in particular hulls, screens, nets, buildings, moorings and signalling systems, against fouling.

Furthermore, the compounds according to the invention, alone or in combinations with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

At certain concentrations and application rates, the active compounds of the formula (I) according to the invention can, if appropriate, also be used as herbicides, for moderating plant growth and for controlling unwanted microorganisms, such as fungi and bacteria, in crop protection and in the protection of materials. If appropriate, they can also be used as intermediates and precursors for the synthesis of further active compounds.

In addition, by the treatment activity it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides inter alia, and also by Aspergillus spec., Penicillium spec., Claviceps purpurea, Stachybotrys spec. inter alia.

Furthermore, the compounds of the formula (I) according to the invention are suitable for suppressing the growth of tumour cells in humans and mammals. This is based on an interaction of the compounds according to the invention with tubulin.

For this purpose, an effective amount of one or more compounds of the formula (I) or pharmacologically acceptable salts thereof may be administered.

The preparation and the use of the active compounds according to the invention is shown in the examples below.

### PREPARATION EXAMPLES

### Example I-1

### 5-Chloro-6-(3,5-dimethyl-1H-pyrazol-1-yl)-7-isopropylaminopyrazolo[1,5-a]pyrimidine-3-carboxylic acid

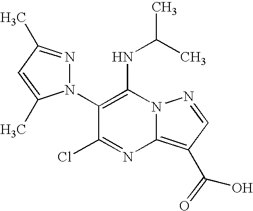

0.49 g of methyl 5-chloro-6-(3,5-dimethyl-1H-pyrazol-1-yl)-7-isopropylaminopyrazolo-[1,5-a]pyrimidine-3-carboxylate were stirred in 15 ml of dioxane, and 15 ml of 2N potassium hydroxide solution were added. The mixture was stirred at room temperature for 15 h. The mixture was then acidified with 1N hydrochloric acid (about pH 1) and extracted with ethyl acetate. The solvent of the organic phase was removed under reduced pressure. This gave 0.32 g of the title compound.

¹H-NMR (DMSO-d₆): δ=8.50 (s, 1H), 7.93 (bd, 1H), 6.12 (s, 1H), 2.19 (s, 3H), 2.11 (s, 3H), 1.12 (d, 3H), 1.09 (d, 3H).

### Example I-2

### N-[5-Chloro-6-(3,5-dimethyl-1H-pyrazol-1-yl)-7-isopropylaminopyrazolo[1,5-a]pyrimidine-3-carbonyl]methanesulphonamide

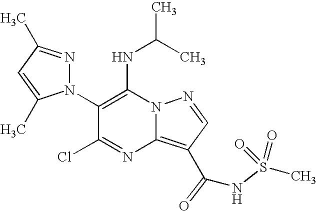

0.32 g of oxalyl chloride and a drop of dimethylformamide were added to a solution of 0.29 g of 5-chloro-6-(3,5-dimethyl-1H-pyrazol-1-yl)-7-isopropylaminopyrazolo[1,5-α]pyrimidine-3-carboxylic acid in 5 ml of dichloromethane, and the mixture was stirred until the evolution of gas had ceased. Stirring was then continued at 40° C. for another 10 min, and the mixture was subsequently concentrated. The residue was again taken up in dichloromethane, and 0.40 g of methanesulphonamide and 4.6 g of phosphoryl chloride were added. The mixture was stirred at 100° C. for 2 h. After cooling to room temperature, the reaction mixture was stirred into water and extracted with ethyl acetate. The organic phase was concentrated, and the residue was digested with ethanol/hexane 1:1. Filtration with suction gave 0.2 g of the title compound as a solid.

¹H-NMR (CD₃CN): δ=8.52 (s, 1H), 7.00 (d, 1H), 6.11 (s, 1H), 3.35 (s, 3H), 2.88-3.03 (m, 1H), 2.24 (s, 3H), 2.14 (s, 3H), 1.18 (d, 3H), 1.12 (d, 3H).

### Example I-14

### 5-Chloro-6-(3,5-dichloropyridin-2-yl)-7-isopropylaminopyrazolo[1,5-a]pyrimidine-3-carboxylic acid

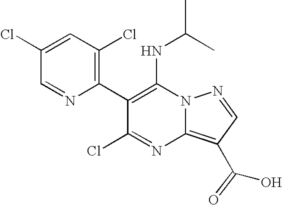

105 mg of methyl 5-chloro-6-(3,5-dichloropyridin-2-yl)-7-isopropylaminopyrazolo-[1,5-a]pyrimidine-3-carboxylate were stirred in 2 ml of dioxane, and 1.27 ml of 2N potassium hydroxide solution were added. The mixture was stirred at 50° C. for 2 h. The mixture was then acidified with 1N hydrochloric acid (about pH 1), and the reaction was evaporated to dryness. The product was extracted from the solid using a mixture of ethyl acetate/methanol. This gave 82 mg of the title compound.

¹H-NMR (DMSO-d₆): δ=11.50-12.50 (bs, 1H), 8.80 (d, 1H), 8.51 (s, 1H), 8.44 (d, 1H), 7.76 (bd, 1H), 3.15-3.25 (m, 1H), 1.09 (d, 3H), 1.06 (d, 3H).

### Example I-15

### N-[5-Chloro-6-(3,5-dichloropyridin-2-yl)-7-isopropylaminopyrazolo[1,5-a]pyrimidine-3-carbonyl]methanesulphonamide

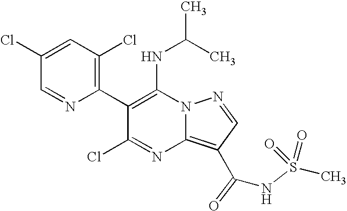

66.5 mg of oxalyl chloride and a drop of dimethylformamide were added to a solution of 70.0 mg of 5-chloro-6-(3,5-dichloropyridin-2-yl)-7-isopropylaminopyrazolo[1,5-a]pyrimidine-3-carboxylic acid in 5 ml of dichloromethane, and the mixture was stirred until the evolution of gas had ceased. The mixture was then stirred at 40° C. for 15 min and subsequently concentrated. 83.1 mg of methanesulphonamide and 1.34 g of phosphoryl chloride were added to the residue. The mixture was stirred at 110° C. for 2 h. After cooling to room temperature, the reaction mixture was added to ice, the mixture was stirred for 30 min and the precipitate was filtered off with suction. Purification was carried out using preparative HPLC. This gave 17 mg of the title compound.

¹H-NMR (DMSO-d₆): δ=10.10 (s, 1H), 8.71 (d, 1H), 8.54 (s, 1H), 8.15 (d, 1H), 7.04 (bd, 1H), 3.36 (s, 3H), 3.19-3.29 (m, 1H), 1.12 (d, 3H), 1.09 (d, 3H).

The compounds of the formula (I) listed in Table 1 below are or were also obtained analogously to the methods indicated above.

TABLE

| | |
|---|---|
| | (I) |
| 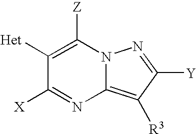 | |

| Ex. | | | | | | |
|---|---|---|---|---|---|---|
| No. | X | Het | Z | R³ | Y | m/z, logP, ¹H-NMR |
| I-1 | Cl | 3,5-(CH₃)₂-pyrazol-1-yl | NH—CH(CH₃)₂ | CO₂H | H | **8.50 (s, 1H), 7.93 (bd, 1H), 6.12 (s, 1 H), |
| | | | | | | 2.19 (s, 3 H), 2.11 (s, 3 H), 1.12 (d, 3H), |
| | | | | | | 1.09 (d, 3H) |
| I-2 | Cl | 3,5-(CH₃)₂-pyrazol-1-yl | NH—CH(CH₃)₂ | CO—NH—SO₂—CH₃ | H | *8.52 (s, 1H), 7.00 (d, 1H), 6.11 (s, 1H), |
| | | | | | | 3.35 (s, 3H), 2.88-3.03 (m, 1H), 2.24 (s, |
| | | | | | | 3H), 2.14 (s, 3H), 1.18 (d, 3H), 1.12 (d, 3H) |
| I-3 | Cl | 3,5-(CF₃)₂-pyrazol-1-yl | NH—CH(CH₃)₂ | CO—NH—SO₂—CH₃ | H | **8.76 (s, 1H), 7.97 (s, 1H), 3.39 (s, 3H), |
| | | | | | | 1.16 (d, 3H), 1.15 (d, 3H) |
| I-4 | Cl | 3,5-(CF₃)₂-pyrazol-1-yl | NH-(R)-CHCH₃—CH(CH₃)₂ | CO—NH—SO₂—CH₃ | H | **8.78 and 8.77 (in each case s, together |
| | | | | | | 1H), 8.01 (s, 1H), 3.39 (s, 3H), 1.11 (dd, |
| | | | | | | 3H), 0.75 (m, 6H) |
| I-5 | Cl | 4-Cl-pyrazol-1-yl | NH—CH(CH₃)₂ | CO—NH—SO₂—CH₃ | H | *8.54 (s, 1H), 7.88 (s, 1H), 7.79 (s, 1H), |
| | | | | | | 7.03-7.14 (m, 1H), 3.35 (s, 3H), 2.91-3.05 |
| | | | | | | (m, 1H), 1.17 (d, 6H) |
| I-6 | Cl | 3,5-(CF₃)₂-pyrazol-1-yl | NH—CH₂—CF₃ | CO₂H | H | *8.54 (s, 1H), 7.49 (s, 1H), 4.20-4.35 (m, |
| | | | | | | 2H) |
| I-7 | Cl | 3,5-(CF₃)₂-pyrazol-1-yl | NH—CH₂—CF₃ | CO—NH—SO₂—CH₃ | H | *8.63 (s, 1H), 7.51 (s, 1H), 4.50-4.62 and |
| | | | | | | 4.33-4.48 (in each case m, together 2H), |
| | | | | | | 3.35 (s, 3H) |
| I-8 | Cl | 3,5-(CF₃)₂-pyrazol-1-yl | 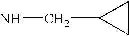 | CO—NH—SO₂—CH₃ | H | *8.58 (s, 1H), 7.46 (s, 1H), 3.35 (s, 3H), 2.76-2.88 (m, 1H), 2.62-2.73 (m, 1H), 1.09 (m, 1H), 0.52 (m, 2H), 0.19 (m, 2H) |
| I-9 | Cl | 4-Cl, 3,5-(CH₃)₂-pyrazol- | NH—CH(CH₃)₂ | CO—NH—SO₂—CH₃ | H | *8.53 (s, 1H), 7.03 (d, 1H), 3.35 (s, 3H), |
| | | 1-yl | | | | 3.00-3.17 (m, 1H), 2.26 (s, 3H), 2.15 (s, |
| | | | | | | 3H), 1.19 (d, 3H), 1.14 (d, 3H) |
| I-10 | Cl | 4-Cl-pyrazol-1-yl | NH—CH(CH₃)₂ | CO—NH—SO₂—CH₂CH₃ | H | *8.53 (s, 1H), 7.88 (s, 1H), 7.79 (s, 1H), |
| | | | | | | 7.07 (d, 1H), 3.52 (q, 2H), 2.91-3.08 (m, |
| | | | | | | 1H), 1.36 (t, 3H), 1.17 (d, 6H) |
| I-11 | Cl | 4-Cl-pyrazol-1-yl | NH—CHCH₃—CH(CH₃)₂ | CO—NH—SO₂—CH₃ | H | *8.54 (s, 1H), 7.88 (s, 1H), 7.81 (s, 1H), |
| | | | | | | 7.07 (d, 1H), 3.35 (s, 3H), 2.35-2.53 (m, |
| | | | | | | 1H), 1.76 (m, 1H), 1.10 (d, 3H), 0.81 (m, |
| | | | | | | 6H) |
| I-12 | Cl | 2,5-(CH₃)₂-thiazol-4-yl | NH—CH(CH₃)₂ | CO₂H | H | *8.42 (s, 1H), 6.78 (d, 1H), 3.40-3.53 (m, |
| | | | | | | 1H), 2.65 (s, 3H), 2.27 (s, 3H), 1.14 (d, |
| | | | | | | 3H), 1.09 (d, 3H) |
| I-13 | Cl | 2,5-(CH₃)₂-thiazol-4-yl | NH—CH(CH₃)₂ | CO—NH—SO₂—CH₃ | H | *8.49 (s, 1H), 6.85 (d, 1H), 3.47-3.60 (m, |
| | | | | | | 1H), 3.35 (s, 3H), 2.65 (s, 3H), 2.28 (s, |
| | | | | | | 3H), 1.16 (d, 3H), 1.10 (d, 3H) |
| I-14 | Cl | 3,5-Cl₂-pyridin-2-yl | NH—CH(CH₃)₂ | CO₂H | H | **11.50-12.50 (bs, 1H), 8.80 (d, 1H), 8.51 |
| | | | | | | (s, 1H), 8.44 (d, 1H), 7.76 (bd, 1H), 3.15- |
| | | | | | | 3.25 (m, 1H), 1.09 (d, 3H), 1.06 (d, 3H) |
| I-15 | Cl | 3,5-Cl₂-pyridin-2-yl | NH—CH(CH₃)₂ | CO—NH—SO₂—CH₃ | H | **10.10 (s, 1H), 8.71 (d, 1H), 8.54 (s, 1H), |
| | | | | | | 8.15 (d, 1H), 7.04 (bd, 1H), 3.36 (s, 3H), |
| | | | | | | 3.19-3.29 (m, 1H), 1.12 (d, 3H), 1.09 (d, |
| | | | | | | 3H) |
| I-16 | Cl | 3,5-F₂-pyridin-2-yl | NH—CH(CH₃)₂ | CO—NH—SO₂—CH₃ | H | **10.12 (s, 1H), 8.58 (d, 1H), 8.54 (s, 1H), |
| | | | | | | 7.67 (td, 1H), 7.04 (bd, 1H), 3.36 (s, 3H), |
| | | | | | | 3.19-3.31 (m, 1H), 1.12 (d, 3H), 1.06 (d, |
| | | | | | | 3H) |
| I-17 | Cl | 1,3,5-(CH₃)₃-pyrazol-4-yl | NH—CH(CH₃)CF₃ | CO₂H | H | *8.56 (s, 1H), 6.47 (bd, 1H), 4.85 (m, 1H) |
| I-18 | Cl | 1,3,5-(CH₃)₃-pyrazol-4-yl | NH—CH(CH₃)CF₃ | CO—NH—SO₂—CH₃ | H | *8.47 (s, 1H), 6.45 (bd, 1H), 4.47 (m, 1H) |
| I-19 | Cl | 3,5-Cl₂-pyridin-4-yl | NH—CH(CH₃)₂ | CO—NH—SO₂—CH₃ | H | **8.88 (s, 2H), 8.69 (s, 1H), 8.06 (bs, 1H), |
| | | | | | | 3.79-3.89 (m, 1H), 1.19 (d, 6H) |
| I-20 | Cl | 3,5-(CF₃)₂-pyrazol-1-yl | NH—CH(CH₃)₂ | CO₂H | H | m/z 457, logP 3.11 |
| I-21 | Cl | 3,5-(CF₃)₂-pyrazol-1-yl | NH-(R)-CHCH₃—CH(CH₃)₂ | CO₂H | H | m/z 485, logP 3.65 and 3.70 (atropisomers) |
| I-22 | Cl | 4-Cl-pyrazol-1-yl | NH—CH(CH₃)₂ | CO₂H | H | **8.57 (s, 1H), 8.40 (s, 1H), 7.99 (s, 1H), |
| | | | | | | 1.12 (d, 6H) |
| I-23 | Cl | 3,5-(CF₃)₂-pyrazol-1-yl | 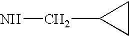 | CO₂H | H | m/z 469, logP 3.22 |
| I-24 | Cl | 4-Cl, 3,5-(CH₃)₂-pyrazol- | NH—CH(CH₃)₂ | CO₂H | H | *8.45 (s, 1H), 6.93 (bd, 1H), 2.95-3.05 (m, |
| | | 1-yl | | | | 1H), 2.26 (s, 3H), 2.15 (s, 3H), 1.17 (d, |
| | | | | | | 3H), 1.12 (d, 3H) |
| I-25 | Cl | 4-Cl-pyrazol-1-yl | NH—CHCH₃—CH(CH₃)₂ | CO₂H | H | m/z 383, logP 2.81 |
| I-26 | Cl | 3,5-F₂-pyridin-2-yl | NH—CH(CH₃)₂ | CO₂H | H | **8.69 (d, 1H), 8.51 (s, 1H), 8.11 (td, 1H), |
| | | | | | | 7.70 (bd, 1H), 1.08 (d, 3H), 1.03 (d, 3H) |
| I-27 | Cl | 3,5-Cl₂-pyridin-4-yl | NH—CH(CH₃)₂ | CO₂H | H | **8.87 (s, 2H), 8.53 (s, 1H), 7.89 (bd, 1H), |
| | | | | | | 3.60-3.70 (m, 1H), 1.12 (d, 6H) |
| I-28 | Cl | pyridin-2-yl | NH—CH(CH₃)₂ | CO₂H | H | **8.72 (ddd, 1H), 8.48 (s, 1H), 7.95 (ddd, |
| | | | | | | 1H), 7.65 (ddd, 1H), 7.57 (bd, 1H), 7.50 |
| | | | | | | (ddd, 1H), 3.00-3.20 (m, 1H), 1.00 (d, 6H) |
| I-29 | Cl | pyridin-2-yl | NH—CH(CH₃)₂ | CO—NH—SO₂—CH₃ | H | **8.73 (ddd, 1H), 8.65 (s, 1H), 7.97 (ddd, |
| | | | | | | 1H), 7.86 (bd, 1H), 7.67 (d, 1H), 7.51 (ddd, |
| | | | | | | 1H), 3.37 (m, 3H), 3.00-3.20 (m, 1H), |
| | | | | | | 1.02 (d, 6H) |
| I-30 | Cl | 3-Cl, 5-CF₃-pyridin-2-yl | NH—CH(CH₃)₂ | CO—NH—SO₂—CH₃ | H | **9.14 (d, 1H), 8.72 (s, 1H), 8.70 (d, 1H), |
| | | | | | | 8.08 (bd, 1H), 3.45 (m, 1H), 3.40 (s, 3H), |
| | | | | | | 1.10 (d, 3H), 1.08 (d, 3H) |
| I-31 | Cl | 3-Cl, 5-CF₃-pyridin-2-yl | NH—CH(CH₃)₂ | CO₂H | H | **9.13 (d, 1H), 8.67 (d, 1H), 8.52 (s, 1H), |
| | | | | | | 7.80 (bd, 1H), 3.24 (m, 1H), 1.08 (d, 3H), |
| | | | | | | 1.05 (d, 3H) |
| I-32 | Cl | 3-Cl-pyridin-2-yl | NH—CH(CH₃)₂ | CO—NH—SO₂—CH₃ | H | **8.73 (dd, 1H), 8.67 (s, 1H), 8.16 (dd, |
| | | | | | | 1H), 8.02 (bd, 1H), 7.63 (dd, 1H), 3.36 (s, |
| | | | | | | 3H), 1.06 (d, 3H), 1.04 (d, 3H) |
| I-33 | Cl | 3-Cl-pyridin-2-yl | NH—CH(CH₃)₂ | CO₂H | H | **8.71 (dd, 1H), 8.51 (s, 1H), 8.14 (dd, |
| | | | | | | 1H), 7.75 (bd, 1H), 7.61 (dd, 1H), 3.05 (m, |
| | | | | | | 1H), 1.04 (d, 3H), 1.03 (d, 3H) |
| I-34 | Cl | 3-F-pyridin-2-yl | NH—CH(CH₃)₂ | CO—NH—SO₂—CH₃ | H | **8.69 (s, 1H), 8.62 (m, 1H), 8.00 (bd, 1H), |
| | | | | | | 7.93 (m, 1H), 7.69 (m, 1H), 3.39 (s, 3H), |
| | | | | | | 3.23 (m, 1H), 1.08 (d, 3H), 1.02 (d, 3H) |
| I-35 | Cl | 3-F-pyridin-2-yl | NH—CH(CH₃)₂ | CO₂H | H | **8.61 (m, 1H), 8.51 (s, 1H), 7.91 (m, 1H), |
| | | | | | | 7.65-7.72 (m, 2H), 1.07 (d, 3H), 0.99 (d, |
| | | | | | | 3H) |
| I-36 | Cl | 3-NO₂-pyridin-2-yl | NH—CH(CH₃)₂ | CO—NH—SO₂—CH₃ | H | **9.07 (dd, 1H), 8.68 (dd, 1H), 8.64 (s, |
| | | | | | | 1H), 8.06 (bd, 1H), 7.92 (dd, 1H), 3.33 (m, |
| | | | | | | 1H), 3.29 (s, 3H), 1.07 (d, 3H), 1.04 (d, 3H) |
| I-37 | Cl | 3-NO₂-pyridin-2-yl | NH—CH(CH₃)₂ | CO₂H | H | **9.06 (dd, 1H), 8.67 (dd, 1H), 8.52 (s, |
| | | | | | | 1H), 7.86-7.98 (m, 2H), 3.23 (m, 1H), |
| | | | | | | 1.05 (d, 3H), 1.03 (d, 3H) |
| I-38 | Cl | 3,5-F₂-pyridin-2-yl | NH—CH(CH₃)₂ | CO—NH—SO₂—C₂H₅ | H | **8.70 (d, 1H), 8.68 (s, 1H), 8.14 (td, 1H), |
| | | | | | | 7.94 (bd, 1H), 3.52 (q, 2H), 3.35 (m, 1H), |
| | | | | | | 1.29 (t, 3H), 1.10 (d, 3H), 1.05 (d, 3H) |
| I-39 | Cl | 3,5-F₂-pyridin-2-yl | NH—CH(CH₃)₂ | 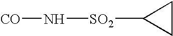 | H | **8.71 (d, 1H), 8.69 (s, 1H), 8.14 (td, 1H), 7.96 (bd, 1H), 3.35 (m, 1H), 1.20-1.24 (m, 2H), 1.12-1.16 (m, 2H), 1.10 (d, 3H), 1.05 (d, 3H) |
| I-40 | Cl | 3,5-F₂-pyridin-2-yl | NH—CH(CH₃)₂ | CO—NH—SO₂—CHF₂ | H | **8.70 (d, 1H), 8.68 (s, 1H), 8.13 (td, 1H), |
| | | | | | | 7.89 (bd, 1H), 7.14 (1, 1H) 3.52 (q, 2H), |
| | | | | | | 3.32 (m, 1H), 1.10 (d, 3H), 1.04 (d, 3H) |
| I-41 | Cl | 3-F-pyridin-4-yl | NH—CH(CH₃)₂ | CO—NH—SO₂—CH₃ | H | **8.77 (s, 1H), 8.66 (s, 1H), 8.61 (d, 1H), |
| | | | | | | 7.80 (bd, 1H), 7.66 (dd, 1H), 3.69 (m, 1H), |
| | | | | | | 3.35 (s, 3H), 1.09 (d, 3H), 1.08 (d, 3H) |
| I-42 | Cl | 3-F-pyridin-4-yl | NH—CH(CH₃)₂ | CO₂H | H | **8.75 (d, 1H), 8.60 (dd, 1H), 8.50 (s, 1H), |
| | | | | | | 7.66 (dd, 1H), 7.56 (bd, 1H), 3.52 (m, 1H), |
| | | | | | | 1.07 (d, 3H), 1.06 (d, 3H) |
| I-43 | Cl | 2,5-(CH₃)₂-thiazol-4-yl | NH—CH(CH₃)CF₃ | CO₂H | H | **8.55 (s, 1H), 7.90 (bd) and 7.69 (bd) |
| | | | | | | (together 1H), 4.19-4.32 (m) and 3.90-4.05 |
| | | | | | | (m) (together 1H), 2.66 (s, 3H), 2.24 (s, |
| | | | | | | 3H), 1.28-1.39 (m, 3H) |
| I-44 | Cl | 2,5-(CH₃)₂-thiazol-4-yl | NH—CH(CH₃)CF₃ | CO—NH—SO₂—CH₃ | H | **8.76 (s, 1H), 8.21 (bd) and 8.00 (bd) |
| | | | | | | (together 1H), 4.35-4.50 (m) and 4.04-4.17 |
| | | | | | | (m) (together 1H), 3.40 (s, 3H), 2.66 (s, |
| | | | | | | 3H), 2.25 (s, 3H), 1.30-1.40 (m, 3H) |
| I-45 | Cl | 2,5-(CH₃)₂-thiazol-4-yl | NH—CH(CH₃)CF₃ | 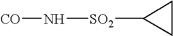 | H | **8.75 (s, 1H), 8.19 (bd) and 7.98 (bd) (together 1H), 4.33-4.50 (m) and 4.04-4.18 (m) (together 1H), 2.66 (s, 3H), 2.25 (s, 3H), 1.30-1.42 (m, 3H), 1.18-1.28 (m, 2H), 1.09-1.18 (m, 2H) |
| I-46 | Cl | 2,5-(CH₃)₂-thiazol-4-yl | NH—CH₂CHF₂ | CO₂H | H | **8.52 (s, 1H), 8.09 (m, 1H), 6.00 (tt, 1H), |
| | | | | | | 3.37-3.57 (m, 2H), 2.65 (s, 3H), 2.24 (s, |
| | | | | | | 3H) |
| I-47 | Cl | 2,5-(CH₃)₂-thiazol-4-yl | NH—CH₂CHF₂ | CO—NH—SO₂—CH₃ | H | **8.70 (s, 1H), 8.32 (m, 1H), 6.03 (tt, 1H), |
| | | | | | | 3.46-3.66 (m, 2H), 3.40 (s, 3H), 2.65 (s, |
| | | | | | | 3H), 2.25 (s, 3H) |
| I-48 | Cl | 2,5-(CH₃)₂-thiazol-4-yl | NH—CH₂CF₃ | CO₂H | H | **8.55 (s, 1H), 8.24 (m, 1H), 4.06-4.19 (m, |
| | | | | | | 1H), 3.87-3.99 (m, 1H), 2.65 (s, 3H), 2.22 |
| | | | | | | (s, 3H) |
| I-49 | Cl | 2,5-(CH₃)₂-thiazol-4-yl | NH—CH₂CF₃ | CO—NH—SO₂—CH₃ | H | **8.74 (s, 1H), 8.45 (m, 1H), 4.16-4.30 (m, |
| | | | | | | 1H), 3.99-4.13 (m, 1H), 3.40 (s, 3H), 2.66 |
| | | | | | | (s, 3H), 2.23 (s, 3H) |
| I-50 | Cl | 3,5-F₂-pyridin-2-yl | NH—CH(CH₃)₂ | 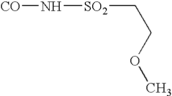 | H | **8.69 (d, 1H), 8.61 (bs, 1H), 8.13 (td, 1H), 7.82 (bd, 1H), 3.65-3.79 (m, 4H), 3.31 (m, 1H), 3.21 (s, 3H), 1.10 (d, 3H), 1.04 (d, 3H) |
| I-51 | Cl | 3,5-F₂-pyridin-2-yl | NH—CH(CH₃)₂ | 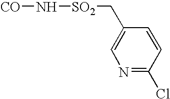 | H | **8.70 (d, 1H), 8.69 (s, 1H), 8.37 (d, 1H), 8.13 (td, 1H), 7.95 (bd, 1H), 7.85 (dd, 1H), 7.52 (d, 1H), 4.95 (s, 2H), 3.38 (m, 1H), 1.10 (d, 3H), 1.05 (d, 3H) |
| I-52 | Cl | 5-Br-pyridin-3-yl | NH—CH(CH₃)₂ | CO—NH—SO₂—CH₃ | H | **8.83 (d, 1H), 8.68 (m, 2H), 8.26 (m, 1H), |
| | | | | | | 7.69 (bd, 1H), 6.63 (bs, 1H), 3.63-3.74 (m, |
| | | | | | | 1H), 3.40 (s, 3H), 1.08 (d, 6H) |
| I-53 | Cl | 5-Br-pyridin-3-yl | NH—CH(CH₃)₂ | CO₂H | H | **8.81 (d, 1H), 8.66 (d, 1H), 8.48 (s, 1H), |
| | | | | | | 8.26 (m, 1H), 7.40 (bd, 1H), 3.57 (s, 3H), |
| | | | | | | 3.39-3.50 (m, 1H), 1.06 (d, 6H) |
| I-54 | Cl | 3-(CH₃O)-pyridin-2-yl | NH—CH(CH₃)₂ | CO—NH—SO₂—CH₃ | H | **10.45 (bs, 1H), 8.67 (s, 1H), 8.32 (d, |
| | | | | | | 1H), 7.86-7.84 (bd, 1H), 7.66 (d, 1H), |
| | | | | | | 7.57 (dd, 1H), 3.83 (s, 3H), 3.42 (s, 3H), |
| | | | | | | 3.19 (m, 1H), 1.04 (d, 3H), 0.92 (d, 3H) |
| I-55 | Cl | 3-(CH₃O)-pyridin-2-yl | NH—CH(CH₃)₂ | CO₂H | H | **12.10 (bs, 1H), 8.48 (s, 1H), 8.30 (d, |
| | | | | | | 1H), 7.63 (d, 1H), 7.54-7.49 (m, 2H), 3.82 |
| | | | | | | (s, 3H), 3.07 (m, 1H), 1.02 (d, 3H), 0.94 (d, |
| | | | | | | 3H) |
| I-56 | Cl | 1,3-(CH₃)₂-4-Cl-pyrazol- | NH—CH(CH₃)₂ | CO—NH—SO₂—CH₃ | H | **8.69 (s, 1H), 8.20 (d, 1H), 3.67 (s, 3H), |
| | | 5-yl | | | | 3.39 (s, 3H), 3.36 (m, 1H), 2.21 (s, 3H), |
| | | | | | | 1.14 (dd, 6H) |
| I-57 | Cl | 1,3-(CH₃)₂-4-Cl-pyrazol- | NH—CH(CH₃)₂ | CO₂H | H | logP 2.20 |
| | | 5-yl | | | | |
| I-58 | Cl | pyrazin-2-yl | NH—CH(CH₃)₂ | CO—NH—SO₂—CH₃ | H | **8.91 (d, 1H), 8.82 (m, 1H), 8.77 (d, 1H), |
| | | | | | | 8.70 (s, 1H), 7.97 (bd, 1H), 3.40 (s, 3H), |
| | | | | | | 3.32-3.40 (m, 1H), 1.05 (d, 6H) |
| I-59 | Cl | pyrazin-2-yl | NH—CH(CH₃)₂ | CO₂H | H | **8.91 (d, 1H), 8.81 (m, 1H), 8.75 (d, 1H), |
| | | | | | | 8.50 (s, 1H), 7.66 (bd, 1H), 3.14-3.20 (m, |
| | | | | | | 1H), 1.03 (d, 6H) |
| I-60 | Cl | 3-F, 5-CF₃-pyridin-2-yl | NH—CH(CH₃)₂ | CO—NH—SO₂—CH₃ | H | **9.05 (d, 1H), 8.71 (s, 1H), 8.53 (dd, 1H), |
| | | | | | | 8.02 (bd, 1H), 3.53-3.61 (m, 1H), 3.40 (s, |
| | | | | | | 3H), 1.09 (dd, 6H) |
| I-61 | Cl | 3-F, 5-CF₃-pyridin-2-yl | NH—CH(CH₃)₂ | CO₂H | H | |
| I-62 | Cl | 3,5-F₂-pyridin-2-yl | NH—CH(CH₃)₂ | 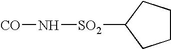 | H | **8.71 (d, 1H), 8.68 (s, 1H), 8.14 (td, 1H), 7.95 (bd, 1H), 4.14 (m, 1H), 3.38 (m, 1H), 1.98-2.04 (m, 4H), 1.58-1.78 (m, 4H), 1.10 (d, 3H), 1.05 (d, 3H) |
| I-63 | Cl | 3-CF₃-pyridin-2-yl | NH—CH(CH₃)₂ | CO—NH—SO₂—CH₃ | H | **9.05 (d, 1H), 8.71 (s, 1H), 8.40 (dd, 1H), |
| | | | | | | 8.09 (bd, 1H), 7.84 (dd, 1H), 6.62 (bs, 1H), |
| | | | | | | 3.40 (s, 3H), 1.03 (dd, 6H) |
| I-64 | Cl | 3-CF₃-pyridin-2-yl | NH—CH(CH₃)₂ | CO₂H | H | **9.03 (d, 1H), 8.51 (s, 1H), 7.77-7.84 (m, |
| | | | | | | 2H), 1.00 (dd, 6H) |

*¹H-NMR (400 MHz, CD₃CN): shift δ in ppm
**¹H-NMR (400 MHz, DMSO-d₆): shift δ in ppm

### Preparation of the Intermediates

### Ethyl[3,5-bis(trifluoromethyl)-1H-pyrazol-1-yl]acetate (known from WO 07/014,290)

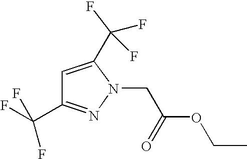

A mixture of 25 g (0.12 mol) of 1,1,1,5,5,5-hexafluoro-2,4-pentanedione and 18.6 g (0.12 mol) of ethyl hydrazinoacetate hydrochloride in 250 ml of ethanol was heated at reflux for 18 h. The solvent was then removed under reduced pressure, and the product was dried under high vacuum. This gave 30.6 g of the title compound.

¹H-NMR (DMSO-d₆): δ=7.64 (s, 1H), 5.40 (s, 2H), 4.19 (q, 2H), 1.20 (t, 3H)

### Dimethyl[3,5-bis(trifluoromethyl)-1H-pyrazol-1-yl]malonate (III-1)

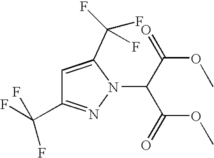

Under argon, 7.2 g of sodium hydride (60% in mineral oil) were added to 151 ml (161 g, 1.793 mol) of dimethyl carbonate, and the mixture was heated at 80° C. At this temperature, a solution of 30.6 g (0.105 mol) of ethyl[3,5-bis(trifluoromethyl)-1H-pyrazol-1-yl]acetate in 30 ml of toluene was slowly added dropwise, and the mixture was stirred at reflux overnight. For workup, the mixture was diluted with about 50 ml of methanol, poured into ice-water and acidified with dilute hydrochloric acid. Extraction with dichloromethane, drying of the organic phase over sodium sulphate, filtration and removal of the solvate gave, after trituration of the solid with petroleum ether, 31 g of dimethyl[3,5-bis(trifluoromethyl)-1H-pyrazol-1-yl]malonate. Yield: 84%.

¹H-NMR (DMSO-d₆): δ=7.77 (s, 1H), 6.80 (s, 1H), 3.80 (s, 6H)

The following further intermediate of the formula (III) for example, was prepared:

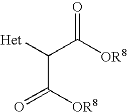

| Ex. No. | Het | R⁸ | ¹H-NMR (CD₃CN) |
|---|---|---|---|
| III-2 | 4-Cl-pyrazol-1-yl | CH₂CH₃ | *7.85 (s, 1H), 7.51 (s, 1H), 5.84 (s, 1H), 4.26 |
| | | | (m, 4H), 1.25 (t, 6H) |

### Methyl 6-(3,5-dimethyl-1H-pyrazol-1-yl)-5,7-dihydroxypyrazolo[1,5-a]pyrimidine-3-carboxylate (IV-1)

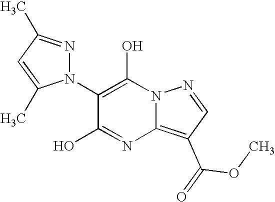

4.73 g of diethyl 2-(3,5-dimethyl-1H-pyrazol-1-yl)malonate (known, for example, from WO 04/011467) and 2.63 g of methyl 3-aminopyrazole-4-carboxylate (known from WO 05/082907) were mixed with 3.79 g of tri-n-butylamine and stirred at 180° C. for 1.5 h; the ethanol released during this reaction was removed by distillation. The mixture was then cooled, and 15 ml of toluene were added dropwise at 140° C. At room temperature, repeatedly, diethyl ether was added to the reaction mixture and the supernatant solution was decanted off. The residue was partitioned between water and dichloromethane. The organic phase was concentrated. This gave 5.7 g of product which was reacted further without further purification.

MS (ES+): 304, logP: 0.41

### Methyl 6-(3,5-dichloropyridin-2-yl)-5,7-dihydroxypyrazolo[1,5-a]pyrimidine-3-carboxylate (IV-2)

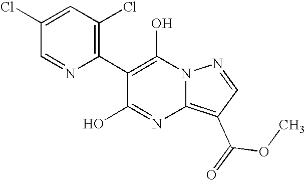

3.34 g of diethyl 2-(3,5-dichloropyridin-2-yl)malonate (known from DE 3820538) and 1.23 g of methyl 3-aminopyrazole-4-carboxylate (known from WO 05/082907) were mixed with 0.73 g of tri-n-butylamine and stirred at 150° C. for 16 h; the ethanol released during this reaction was removed by distillation. The mixture was then cooled, and 50 ml of ethyl acetate were added dropwise. The precipitate formed was filtered off with suction and discounted. The filtrate was extracted with 50 ml of 1N NaOH. The basic solution was adjusted with 1N HCl initially to pH 6 and then to pH 1 and in each case washed with ethyl acetate. The HCl-acidic aqueous phase was concentrated to dryness, and the product was extracted from the solid using a mixture of dichloromethane and MeOH. This gave 890 mg of product.

MS (ES+): 355, logP: 0.74

### Methyl 6-(3,5-dichloropyridin-4-yl)-5,7-dihydroxypyrazolo pyrimidine-3-carboxylate (IV-8)

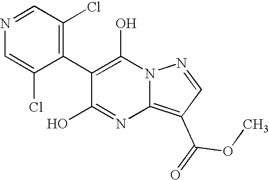 [0565] a) 1.66 g of sodium hydride (95%) were added to 80 ml of THF. 1.59 g of malononitrile were added, and the mixture was stirred at room temperature for 1 h. 6 g of 3,5-dichloro-5-iodopyridine (CAS 343781-41-9), 78 mg of palladium(II) chloride and 369 mg of tricyclohexylphosphine were then added. The reaction mixture was stirred at 60° C. for 16 h. The mixture was diluted with ethyl acetate and extracted with 1N aqueous sodium hydroxide solution. The aqueous phase was acidified with hydrochloric acid and extracted with ethyl acetate. The organic phase obtained in this manner was concentrated. This gave 1.1 g of 2-(3,5-dichloropyridin-4-yl)malononitrile.

¹H-NMR (DMSO-d₆): δ=8.11 (s), MS (ES+): 212, logP: 0.90
b) 1.65 g of 2-(3,5-dichloropyridin-4-yl)malononitrile were added to 4 ml of concentrated sulphuric acid and 0.28 ml of water. The mixture was stirred at 50° C. for 5 h. After cooling to room temperature, the reaction mixture was poured onto ice and extracted with ethyl acetate. The organic phase was concentrated. This gave 220 mg of 2-(3,5-dichloropyridin-4-yl)malonamide.

¹H-NMR (DMSO-d₆): δ=8.56 (s, 2H), 5.00 (s, 1H).
c) 220 mg of 2-(3,5-dichloropyridin-4-yl)malonamide, 100 mg of methyl 3-aminopyrazol-4-carboxylate (known from WO 05/082907) and 47 mg of tri-n-butylamine in 10 ml of 1,2-dichlorobenzene were stirred at 190° C. for 3 h. The reaction mixture was allowed to cool to room temperature. The mixture was stirred with cyclohexane, and the precipitate formed was filtered off with suction. This gave 120 mg of the title compound.

¹H-NMR (DMSO-d₆): δ=8.48 (s, 2H), 7.81 (s, 1H), 3.77 (s, 3H).

The following further intermediates of the formula (IV), for example, were prepared:

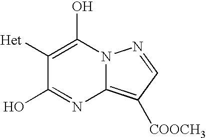

| Ex. No. | Het | m/z, logP, ¹H-NMR (DMSO-d₆) |
|---|---|---|
| IV-3 | 3,5-(CF₃)₂-pyrazol-1-yl | m/z 412, logP 1.37 |
| IV-4 | 4-Cl-pyrazol-1-yl | m/z 310, logP 0.43 |
| IV-5 | 2,5-(CH₃)₂-thiazol-4-yl | m/z 321, logP 0.43 |
| IV-6 | 3,5-F₂-pyridin-2-yl | in TFA-d: 8.87 (s, 1H), 8.67 (s, 1H), 8.23 (m, |
| | | 1H), 4.22 (s, 3H) |
| IV-7 | 1,3,5-(CH₃)₃-pyrazol-4-yl | |
| IV-8 | 3,5-Cl₂-pyridin-4-yl | 8.48 (s, 2H), 7.81 (s, 1H), 3.77 (s, 3H) |
| IV-9 | pyridin-2-yl | 9.38 (d, 1H), 8.92 (s, 1H), 8.60 (dd, 1H), 8.48 |
| | | (ddd, 1H), 7.74 (ddd, 1H), 4.23 (s, 3H) |
| IV-10 | 3-Cl, 5-CF₃-pyridin-2-yl | 8.86 (bs, 1H), 8.28 (s, 1H), 7.89 (s, 1H), 3.79 (s, |
| | | 3H) |
| IV-11 | 3-Cl-pyridin-2-yl | 8.65 (d, 1H), 8.44 (d, 1H), 7.90 (s, 1H), 7.67 (m, |
| | | 1H), 3.78 (s, 3H) |
| IV-12 | 3-F-pyridin-2-yl | 8.49 (bd, 1H), 8.22 (t, 1H), 7.92 (s, 1H), 7.66 (m, |
| | | 1H), 3.79 (s, 3H) |
| IV-13 | 3-NO₂-pyridin-2-yl | in TFA-d: 9.03 (dd, 1H), 8.97 (dd, 1H), 8.83 (s, |
| | | 1H), 8.02 (dd, 1H), 4.22 (s, 3H) |
| IV-14 | 3-F-pyridin-4-yl | 8.59 (d, 1H), 8.35 (d, 1H), 8.01 (m, 1H), 7.86 (s, |
| | | 1H), 3.78 (s, 3H) |
| IV-15 | 5-Br-pyridin-3-yl | logP 0.78 |
| IV-16 | 3-(CH₃O)-pyridin-2-yl | |
| IV-17 | 1,3-(CH₃)₂-4-Cl-pyrazol-5-yl | logP 0.59 |
| IV-18 | pyrazin-2yl | |
| IV-19 | 3-F, 5-CF₃-pyridin-2-yl | logP 0.91 |
| IV-20 | 3-CF₃-pyridin-2-yl | logP 0.65 |

### Methyl 5,7-dichloro-6-(3,5-dimethyl-1H-pyrazol-1-yl)pyrazolo[1,5-a]pyrimidine-3-carboxylate (V-1)

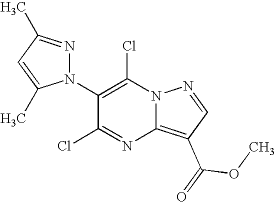

3 g of methyl 6-(3,5-dimethyl-1H-pyrazol-1-yl)-5,7-dihydroxypyrazolo[1,5-a]pyrimidine-3-carboxylate were dissolved in 9.1 g of phosphoryl chloride and stirred at 120° C. for 2 h until the evolution of gas had ceased. 2.06 g of phosphorus pentachloride were then added, and stirring at 120° C. was continued for 2 h. After subsequent cooling, the reaction mixture was poured into icewater and extracted with ethyl acetate. The solvent of the organic phase was removed under reduced pressure. The residue was purified chromatographically (silica gel, cyclohexane/ethyl acetate 1:1). This gave 0.58 g of the title compound.

MS (ES+): 340, logP: 2.16

### Methyl 5,7-dichloro-6-(3,5-dichloropyridin-2-yl)pyrazolo[1,5-a]pyrimidine-3-carboxylate (V-2)

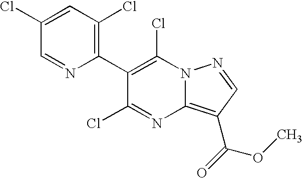

7.4 g of phosphoryl chloride and 292 mg of tri-n-butylamine were added to 600 mg of methyl 6-(3,5-dichloropyridin-2-yl)-5,7-dihydroxypyrazolo[1,5-a]pyrimidine-3-carboxylate, and the mixture was stirred at 120° C. for 16 h. 165 mg of phosphorus pentachloride were then added, and stirring at 120° C. was continued for 4 h. After subsequent cooling, the reaction mixture was concentrated, taken up in ethyl acetate and washed with saturated sodium bicarbonate solution and saturated sodium chloride solution. The organic phase was dried and the solvent was removed under reduced pressure. This gave 915 mg of crude product which were reacted without further purification.

MS (ES+): 391, logP: 3.12

The following further intermediates of the formula (V), for example, were prepared:

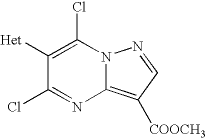

| Ex. No. | Het | m/z, logP, ¹H-NMR (DMSO-d₆) |
|---|---|---|
| V-3 | 3,5-(CF₃)₂-pyrazol-1-yl | m/z 448, logP 3.64 |
| V-4 | 4-Cl-3,5-(CH₃)₂-pyrazol-1-yl | 8.11 (s, 1H), 3.74 (s, 3H), 2.16 (s, 3H), 1.99 (s, 1H) |
| V-5 | 4-Cl-pyrazol-1-yl | m/z 346, logP 2.50 |
| V-6 | 2,5-(CH₃)₂-thiazol-4-yl | 8.77 (s, 1H), 3.87 (s, 3H), 2.66 (s, 3H), 2.30 (s, 3H) |
| V-7 | 3,5-F₂-pyridin-2-yl | 8.82 (s, 1H), 8.72 (d, 1H), 8.22 (dd, 1H), 3.88 (s, |
| | | 3H) |
| V-8 | 1,3,5-(CH₃)₃-pyrazol-4-yl | m/z 354, logP 1.88 |
| V-9 | 3,5-Cl₂-pyridin-4-yl | 8.96 (s, 2H), 8.89 (s, 1H), 3.89 (s, 3H) |
| V-10 | pyridin-2-yl | 8.79 (s, 1H), 8.77-8.78 (m, 1H), 8.03 (ddd, 1H), 7.67 |
| | | (ddd, 1H), 7.56 (ddd, 1H), 3.88 (s, 3H) |
| V-11 | 3-Cl, 5-CF₃-pyridin-2-yl | 9.21 (d, 1H), 8.85 (s, 1H), 8.79 (d, 1H), 3.89 (s, 3H) |
| V-12 | 3-Cl-pyridin-2-yl | 8.83 (s, 1H), 8.77 (dd, 1H), 8.22 (dd, 1H), 7.69 (dd, |
| | | 1H), 3.89 (s, 3H) |
| V-13 | 3-F-pyridin-2-yl | 8.81 (s, 1H), 8.68 (dd, 1H), 8.01 (m, 1H), 7.75 (m, |
| | | 1H), 3.88 (s, 3H) |
| V-14 | 3-NO₂-pyridin-2-yl | 9.17 (dd, 1H), 8.84 (s, 1H), 8.83 (dd, 1H), 8.01 (dd, |
| | | 1H), 3.89 (s, 3H) |
| V-15 | 3-F-pyridin-4-yl | 8.85 (d, 1H), 8.82 (s, 1H), 8.69 (dd, 1H), 7.68 (dd, |
| | | 1H), 3.88 (s, 3H) |
| V-16 | 5-Br-pyridin-3-yl | 8.88 (d, 1H), 8.81 (s, 1H), 8.70 (d, 1H), 8.27 (m, |
| | | 1H), 3.88 (s, 3H) |
| V-17 | 3-(CH₃O)-pyridin-2-yl | 8.79 (s, 1H), 8.36 (d, 1H), 7.73 (d, 1H), 7.61 (dd, |
| | | 1H), 3.89 (s, 3H), 3.84 (s, 3H) |
| V-18 | 1,3-(CH₃)₂-4-Cl-pyrazol-5-yl | logP 2.67 |
| V-19 | pyrazin-2yl | 8.93 (m, 1H), 8.89 (m, 1H), 8.84 (m, 1H), 8.82 (s, |
| | | 1H), 3.88 (s, 3H) |
| V-20 | 3-F, 5-CF₃-pyridin-2-yl | in CD₃CN: 8.97 (d, 1H), 8.69 (s, 1H), 8.13 (dd, 1H), |
| | | 3.91 (s, 3H) |
| V-21 | 3-CF₃-pyridin-2-yl | logP 2.61 |

### Methyl 5-chloro-6-(3,5-dimethyl-1H-pyrazol-1-yl)-7-isopropylaminopyrazolo-[1,5-a]pyrimidine-3-carboxylate (VI-1)

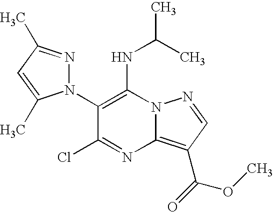

0.58 g of methyl 5,7-dichloro-6-(3,5-dimethyl-1H-pyrazol-1-yl)pyrazolo[1,5-a]pyrimidine-3-carboxylate was dissolved in 20 ml of acetonitrile, and 0.11 g of isopropylamine and 0.28 g of potassium carbonate were added. The mixture was stirred at room temperature for 15 h. The mixture was then acidified with 1N hydrochloric acid (about pH 1) and extracted with ethyl acetate. The solvent of the organic phase was removed under reduced pressure. This gave 0.52 g of the title compound.

¹H-NMR (DMSO-d₆): δ=8.56 (s, 1H), 7.99 (m, 1H), 6.12 (s, 1H), 3.82 (s, 3H), 2.19 (s, 3H), 2.11 (s, 3H), 1.12 (d, 3H), 1.10 (d, 3H).

### Methyl 6-[3,5-bis(trifluoromethyl)-1H-pyrazol-1-yl]-5-chloro-7-[(2,2,2-trifluoroethyl)amino]-pyrazolo[1,5-a]pyrimidine-3-carboxylate (VI-2)

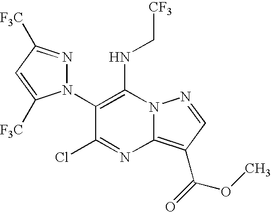

1 g of methyl 6-[3,5-bis(trifluoromethyl)-1H-pyrazol-1-yl]-5,7-dichloropyrazolo[1,5-a]pyrimidine-3-carboxylate was dissolved in 1 ml of acetonitrile, and 0.24 g of 2,2,2-trifluoroethylamine and 0.37 g of potassium carbonate were added. The mixture was stirred at room temperature for 15 h and then stirred under reflux for 8 h. The mixture was then acidified with 1N hydrochloric acid (about pH 1) and extracted with ethyl acetate. The solvent of the organic phase was removed under reduced pressure. This gave 0.53 g of the title compound.

MS (ES+): 511, logP: 3.79

### Methyl 5-chloro-6-(3,5-dichloropyridin-2-yl)-7-isopropylaminopyrazolo[1,5-a]pyrimidine-3-carboxylate (VI-3)

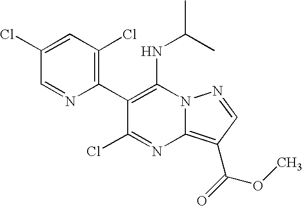

0.91 g of methyl 5,7-dichloro-6-(3,5-dichloropyridin-2-yl)pyrazolo[1,5-a]pyrimidine-3-carboxylate was dissolved in 50 ml of acetonitrile, and 0.10 g of isopropylamine and 0.26 g of potassium carbonate were added. The mixture was stirred at room temperature for 6 h. The reaction mixture was then filtered, and the filtrate was concentrated. Purification was carried out by preparative HPLC. This gave 110 mg of the title compound.

¹H-NMR (DMSO-d₆): δ=8.79 (d, 1H), 8.56 (s, 1H), 8.43 (d, 1H), 7.81 (bd, 1H), 3.82 (s, 3H), 3.70-3.80 (m, 1H), 1.06 (d, 3H), 1.08 (d, 3H)

The following further intermediates of the formula (VI), for example, were prepared:

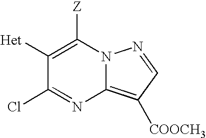

| Ex. | | | |
|---|---|---|---|
| No. | Het | Z | m/z, logP, ¹H-NMR (DMSO-d₆) |
| VI-4 | 3,5-(CF₃)₂-pyrazol- | NH—CH(CH₃)₂ | m/z 471; 8.62 (s, 1H), 7.96 (s, 1H), 3.83 |
| | 1-yl | | (s, 3H), 1.16 (d, 3H), 1.13 (d, 3H) |
| VI-5 | 3,5-(CF₃)₂-pyrazol- | NH-(R)-CHCH₃—CH(CH₃)₂ | m/z 499, logP 4.51 and 4.58 |
| | 1-yl | | (atropisomers) |
| VI-6 | 3,5-(CF₃)₂-pyrazol- 1-yl | 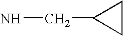 | m/z 483, logP 4.01 |
| VI-7 | 4-Cl-3,5-(CH₃)₂- | NH—CH(CH₃)₂ | 8.57 (s, 1H), 8.07 (bd, 1H), 3.82 (s, 3H), |
| | pyrazol-1-yl | | 2.22 (s, 3H), 2.14 (s, 3H), 1.14 (d, 3H), |
| | | | 1.10 (d, 3H) |
| VI-8 | 4-Cl-pyrazol-1-yl | NH—CH(CH₃)₂ | 8.58 (s, 1H), 8.33 (s, 1H), 8.10 (d, 1H), |
| | | | 3.82 (s, 3H), 2.68-2.80 (m, 1H), 1.13 (d, |
| | | | 6H) |
| VI-9 | 4-Cl-pyrazol-1-yl | NH—CHCH₃—CH(CH₃)₂ | m/z 397, logP 3.59 |
| VI-10 | 2,5-(CH₃)₂-thiazol- | NH—CH(CH₃)₂ | 8.52 (s, 1H), 7.56 (m, 1H), 3.81 (s, 3H), |
| | 4-yl | | 3.20-3.35 (m, 1H), 2.64 (s, 3H), 2.25 (s, |
| | | | 3H), 1.10 (d, 3H), 1.05 (d, 3H) |
| VI-11 | 3,5-F₂-pyridin-2-yl | NH—CH(CH₃)₂ | m/z 382, logP 2.76 |
| VI-12 | 1,3,5-(CH₃)₃- | NH—CH(CH₃)CF₃ | m/z 431, logP 2.54 |
| | pyrazol-4-yl | | |
| VI-13 | 3,5-Cl₂-pyridin-4- | NH—CH(CH₃)₂ | 8.87 (s, 2H), 8.59 (s, 1H), 7.95 (bd, 1H), |
| | yl | | 3.82 (s, 3H), 3.61-3.71 (m, 1H), 1.12 (d, |
| | | | 6H) |
| VI-14 | pyridin-2-yl | NH—CH(CH₃)₂ | 8.72 (ddd, 1H), 8.54 (s, 1H), 7.95 (ddd, |
| | | | 1H), 7.61-7.67 (m, 2H), 7.50 (ddd, 1H), |
| | | | 3.81 (s, 3H), 3.05-3.15 (m, 1H), 1.00 (d, |
| | | | 6H) |
| VI-15 | 3-Cl, 5-CF₃- | NH—CH(CH₃)₂ | 9.21 (d, 1H), 8.85 (s, 1H), 8.79 (d, 1H), |
| | pyridin-2-yl | | 3.89 (s, 3H) |
| VI-16 | 3-Cl-pyridin-2-yl | NH—CH(CH₃)₂ | 8.83 (s, 1H), 8.77 (dd, 1H), 8.22 (dd, |
| | | | 1H), 7.69 (dd, 1H), 3.89 (s, 3H) |
| VI-17 | 3-F-pyridin-2-yl | NH—CH(CH₃)₂ | 8.81 (s, 1H), 8.68 (dd, 1H), 8.01 (m, 1H), |
| | | | 7.75 (m, 1H), 3.88 (s, 3H) |
| VI-18 | 3-NO₂-pyridin-2-yl | NH—CH(CH₃)₂ | 9.17 (dd, 1H), 8.84 (s, 1H), 8.83 (dd, |
| | | | 1H), 8.01 (dd, 1H), 3.89 (s, 3H) |
| VI-19 | 3-F-pyridin-4-yl | NH—CH(CH₃)₂ | 8.85 (d, 1H), 8.82 (s, 1H), 8.69 (dd, 1H), |
| | | | 7.68 (dd, 1H), 3.88 (s, 3H) |
| VI-20 | 2,5-(CH₃)₂-thiazol- | NH—CH(CH₃)CF₃ | 8.61 (s, 1H), 7.96 (bd) and 7.75 (bd) |
| | 4-yl | | (together 1H), 4.19-4.31 (m) and 3.90- |
| | | | 4.05 (m) (together 1H), 3.83 (s, 3H), 2.66 |
| | | | (s, 3H), 2.24 (s, 3H), 1.29-1.40 (m, 3H) |
| VI-21 | 2,5-(CH₃)₂-thiazol- | NH—CH₂CHF₂ | 8.57 (s, 1H), 8.13 (m, 1H), 6.00 (tt, 1H), |
| | 4-yl | | 3.82 (s, 3H), 3.37-3.57 (m, 2H), 2.65 (s, |
| | | | 3H), 2.24 (s, 3H) |
| VI-22 | 2,5-(CH₃)₂-thiazol- | NH—CH₂CF₃ | 8.61 (s, 1H), 8.29 (m, 1H), 4.06-4.19 (m, |
| | 4-yl | | 1H), 3.87-3.99 (m, 1H), 3.82 (s, 3H), |
| | | | 2.65 (s, 3H), 2.22 (s, 3H) |
| VI-23 | 5-Br-pyridin-3-yl | NH—CH(CH₃)₂ | 8.81 (d, 1H), 8.66 (d, 1H), 8.54 (s, 1H), |
| | | | 8.26 (m, 1H), 7.45 (bd, 1H), 3.81 (s, 3H), |
| | | | 3.40-3.51 (m, 1H), 1.06 (d, 6H) |
| VI-24 | 3-(CH₃O)-pyridin- | NH—CH(CH₃)₂ | 8.53 (s, 1H), 8.30 (d, 1H), 7.63 (d, 1H), |
| | 2-yl | | 7.57-7.52 (m, 2H), 3.82 (s, 3H), 3.79 (s, |
| | | | 3H), 3.07 (m, 1H), 1.02 (d, 3H), 0.96 (d, |
| | | | 3H) |
| VI-25 | 1,3-(CH₃)₂-4-Cl- | NH—CH(CH₃)₂ | 8.56 (s, 1H), 7.95 (d, 1H), 3.82 (s, 3H), |
| | pyrazol-5-yl | | 3.66 (s, 3H), 3.25 (m, 1H), 2.21 (s, 3H), |
| | | | 1.12 (dd, 6H) |
| VI-26 | pyrazin-2-yl | NH—CH(CH₃)₂ | 8.91 (m, 1H), 8.81 (m, 1H), 8.75 (m, |
| | | | 1H), 8.56 (s, 1H), 7.71 (bd, 1H), 3.82 (s, |
| | | | 3H), 3.14-3.20 (m, 1H), 1.03 (d, 6H) |
| VI-27 | 3-F, 5-CF₃- | NH—CH(CH₃)₂ | in CD₃CN: 8.92 (d, 1H), 8.45 (s, 1H), |
| | pyridin-2-yl | | 8.05 (dd, 1H), 6.92 (m, 1H), 3.86 (s, 3H), |
| | | | 3.19-3.25 (m, 1H), 1.07 (dd, 6H) |
| VI-28 | 3-CF₃-pyridin-2-yl | NH—CH(CH₃)₂ | 9.03 (dd, 1H), 8.57 (s, 1H), 7.80-7.87 (m, |
| | | | 2H), 3.82 (s, 3H), 1.00 (dd, 6H) |

The logP values given in the tables and preparation examples above were determined in accordance with EEC Directive 79/831 Annex V.A8 by HPLC (High Performance Liquid Chromatography) on a reverse-phase column (C 18). Temperature: 43° C.

The determination was carried out by LC-MS at pH 2.7 using 0.1% aqueous formic acid and acetonitrile (containing 0.1% formic acid) as mobile phases; linear gradient from 10% acetonitrile to 95% acetonitrile.

Calibration was carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

From the mass spectra obtained, the m/z of the [M+H]⁺ peaks are stated.

### USE EXAMPLES

### Example 1

### Phaedon Test (Spray Treatment)

Solvents: 78 parts by weight of acetone
1.5 parts by weight of dimethylformamide

Emulsifier: 0.5 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Discs of chinese cabbage (Brassica pekinensis) are sprayed with an active compound preparation of the desired concentration and, after drying, populated with larvae of the mustard beetle (Phaedon cochleariae).

After the desired period of time, the effect in % is determined. 100% means that all beetle larvae have been killed, 0% means that none of the beetle larvae have been killed.

In this test, for example, the following compounds of the preparation examples show, at an application rate of 500 g/ha, an efficacy of ≧80%: I-3, I-4, I-6, I-8, I-10, I-14, I-15, I-16, I-19, I-30, I-32, I-34, I-38, I-39, I-45, I-50, I-51, I-60, I-63.

### Example 2

### Myzus Test (Spray Treatment)

Solvents: 78 parts by weight of acetone
1.5 parts by weight of dimethylformamide

Emulsifier: 0.5 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Discs of chinese cabbage (Brassica pekinensis) which are infested by all stages of the green peach aphid (Myzus persicae) are sprayed with an active compound preparation of the desired concentration.

After the desired period of time, the effect in % is determined. 100% means that all aphids have been killed; 0% means that none of the aphids have been killed.

In this test, for example, the following compounds of the preparation examples show, at an application rate of 500 g/ha, an efficacy of ≧80%: I-2, I-3, I-4, I-5, I-9, I-10, I-12, I-13, I-15, I-16, I-18, I-19, I-29, I-30, I-32, I-34, I-36, I-38, I-39, I-40, I-41, I-43, I-44, I-45, I-47, I-48, I-49, I-50, I-51, I-54, I-58, I-60, I-62, I-63.

### Example 3

### Tetranychus Test, Op-Resistant (Spray Treatment)

Solvents: 78 parts by weight of acetone
1.5 parts by weight of dimethylformamide

Emulsifier: 0.5 part by weight of alkylaryl polyglycol ether

To produce a suitable preparation of active compound, 1 part by weight of active compound is mixed with the stated amounts of solvents and emulsifier, and the concentrate is diluted with emulsifier-containing water to the desired concentration.

Discs of bean leaves (Phaseolus vulgaris) infested by all stages of the greenhouse red spider mite (Tetranychus urticae) are sprayed with an active compound preparation of the desired concentration.

After the desired period of time, the effect in % is determined. 100% means that all spider mites have been killed; 0% means that none of the spider mites have been killed.

In this test, for example, the following compounds of the preparation examples show, at an application rate of 500 g/ha, an efficacy of ≧80%: I-2, I-19, I-29, I-32, I-38, I-40, I-49, I-51, I-56.

### Example 4

### Meloidogyne Test (Spray Treatment)

Solvent: 80 parts by weight of acetone

To produce a suitable preparation of active compound, 1 part by weight of active compound is mixed with the stated amount of water, and the concentrate is diluted with water to the desired concentration.

Vessels are filled with sand, solution of active compound, Meloidogyne incognita egg/larvae suspension and lettuce seeds. The lettuce seeds germinate and the plants develop. On the roots, galls are formed.

After the desired period of time, the nematicidal effect is determined in % by the gall formation.

100% means that no galls were found; 0% means that the number of galls on the treated plants corresponds to that of the untreated control.

In this test, for example, the following compounds of the preparation examples show, at a concentration of 20 ppm or 40 ppm*, an efficacy of ≧80%: I-2, I-5*, I-7, I-15, I-16, I-36.

### Example 5

### Lucilia cuprina Test

Solvent: dimethyl sulphoxide

To produce a suitable preparation of active compound, 1 part by weight of active compound is mixed with the stated amount of water, and the concentrate is diluted with water to the desired concentration.

Vessels containing horse meat which had been treated with the preparation of active compound of the desired concentration are populated with Lucilia cuprina larvae.

After the desired period of time, the kill in % is determined. 100% means that all larvae have been killed; 0% means that none of the larvae have been killed.

In this test, for example, the following compounds of the preparation examples show, at an application rate of 100 ppm, an efficacy of ≧80%: I-4, I-16, I-19

### Example 6

### Boophilus microplus Test (Injection)

Solvent: dimethyl sulphoxide

To produce a suitable preparation of active compound, 1 part by weight of active compound is mixed with the stated amount of solvent, and the concentrate is diluted with solvent to the desired concentration.

The solution of active compound is injected into the abdomen (Boophilus microplus), the animals are transferred into dishes and stored in a climatized room.

After the desired period of time, the effect in % is determined. 100% means that none of the ticks has laid any fertile eggs.

In this test, for example, the following compounds of the preparation examples show, at an application rate of 20 μg/animal, an effect of ≧80%: I-4

### Example 7

### Sphaerotheca Test (Cucumber)/Protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the active compound preparation at the stated application rate. The plants are inoculated 1 day after the treatment with a spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, for example, the following compounds of the preparation examples show, at a concentration of 500 ppm, an effectiveness of ≧70%: I-4, I-15, I-16, I-34.

### Example 8

### Botrytis Test (Cucumber)/Protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with active compound preparation at the stated application rate. The plants are inoculated 1 day after the treatment with a spore suspension of Botrytis cinerea and then remain at 100% relative humidity and 20° C. for 48 h. The plants then remain at 96% relative atmospheric humidity and a temperature of 13° C.

Evaluation is carried out 5-6 days after the inoculation. 0% means an efficacy which corresponds to that of the control, whereas an efficacy of 100% means that no infection is observed.

In this test, for example, the following compounds of the preparation examples show, at a concentration of 500 ppm, an effectiveness of ≧70%: I-1, I-2, I-3, I-4, I-5, I-8, I-9, I-10, I-11, I-12, I-13, I-14, I-15, I-16, I-18, I-22, I-26, I-31, I-32, I-33, I-34, I-38, I-39, I-43, I-44, I-45, I-46, I-50.

### Example 9

### Puccinia Test (Wheat)/Protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young wheat plants are sprayed with the active compound preparation at the stated application rate. The plants are inoculated with a spore suspension of Puccinia recondita 1 day after treatment and then remain at 100% relative humidity and 20° C. for 48 h. The plants then remain at 80% relative atmospheric humidity and a temperature of 20° C.

Evaluation is carried out 8-10 days after the inoculation. 0% means an efficacy which corresponds to that of the control, whereas an efficacy of 100% means that no infection is observed.

In this test, for example, the following compounds of the preparation examples show, at a concentration of 500 ppm, an effectiveness of ≧70%: I-3, I-4, I-10, I-11, I-15, I-16, I-26, I-31, I-32, I-39, I-41, I-45.

### Example 10

### Activity Increase by Ammonium/Phosphonium Salts in Combination with Penetrants Myzus persicae Test

Solvent: 7 parts by weight of dimethylformamide

Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration. If the addition of ammonium salts or ammonium salts and penetrants is required, these are in each case added in a concentration of 1000 ppm after dilution to the finished solution of the preparation.

Bell pepper plants (Capsicum annuum) which are heavily infested by the green peach aphid (Myzus persicaei) are treated by being sprayed with the active compound preparation at the desired concentration.

After the desired period of time, the kill in % is determined. 100% means that all animals have been killed; 0% means that none of the animals have been killed.

In this test, for example, the following compounds of the preparation examples show good effectiveness: see table

TABLE

| | | | | +AS + RME |
|---|---|---|---|---|
| Active | Concentration | Kill (%) | +AS | in each case |
| compound | (ppm) | after 6 d | 1000 ppm | 1000 ppm |
| I-32 | 20 | 10 | 60 | 90 |

### Example 11

### Increase of Penetration into the Plant by Ammonium or Phosphonium Salts and Synergistic Increase of Penetration into the Plant by Ammonium/Phosphonium Salts in Combination with Penetrants

In this test, the penetration of active compounds through enzymatically isolated cuticles of pear tree leaves was measured.

Use was made of leaves which, fully developed, were cut from pear trees. The cuticles were isolated by
initially filling leaf discs punched out and stained with dye on the underside by vacuum infiltration with a pectinase solution (0.2 to 2% strength) buffered to a pH between 3 and 4,
then sodium azide was added and
allowing the leaf discs treated in this manner to stand until the original leaf structure has dissolved and the non-cellular cuticles have detached.

Only the cuticles, free from hairs and stoma, of the upper sides of the leaves were then used. They were washed repeatedly alternating with water and a buffer solution of pH 7. The clean cuticles obtained were then mounted on Teflon plates and smoothed and dried with a gentle stream of air.

In the next step, the cuticle membranes obtained in this manner were placed into stainless steel diffusion cells (=transport chambers) for membrane transport studies. To this end, the cuticles were placed with a pincette into the centre of the edges, coated with silicone fat, of the diffusion cells and closed with a ring, which had also been treated with fat. The arrangement was chosen such that the morphological outside of the cuticles was facing outwards, i.e. exposed to air, whereas the original inside was facing the interior of the diffusion cells.

The diffusion cells were filled with a 30% strength ethylene glycol/water solution. To determine the penetration, in each case 5 μl of the spray liquor of the composition below were applied to the outside of the cuticles. The spray liquor was prepared using local tap water of medium hardness.

After the spray liquors had been applied, the water was allowed to evaporate and the chambers were inverted and placed into thermostated taps in which temperature and atmospheric humidity over the cuticles could be adjusted using a gentle stream of air onto the cuticles with the spray coating (20° C., 60% rh). At regular intervals, an autosampler took aliquots and the active compound content was determined by HPLC.

The test results are shown in the table below. The stated numbers are average values of 8 to 10 measurements. It is clearly evident that, together with ammonium sulphate and RME, penetration is improved considerably.

| | TABLE | | |
|---|---|---|---|
| | Penetration after 24 h at pH 8/% | | |
| | | EC + RME | EC + RME (1 g/l) + |
| Active compound | EC | (1 g/l) | AS (1 g/l) |
| Example I-16 | 0.2 | 0.8 | 7 |
| 500 ppm in DMF/emulsifer | | | |
| W 7:1 (w/w) | | | |

RME = rapeseed oil methyl ester (formulated as 500 EW, concentration stated in g of active compound/l)
AS = ammonium sulphate
EC = emulsifiable concentrate

1. Heterocyclic carboxylic acid derivatives of the formula (I), 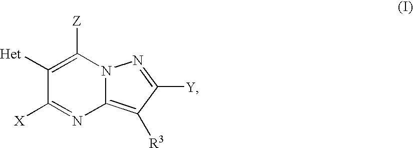
in which the symbols have the following meaning:
X represents hydrogen, halogen, cyano, hydroxyl, optionally substituted alkyl, haloalkyl, optionally substituted alkoxy, optionally substituted phenyl, optionally substituted alkylthio, optionally substituted alkylsulphinyl or optionally substituted alkylsulphonyl;
Het represents one of the radicals 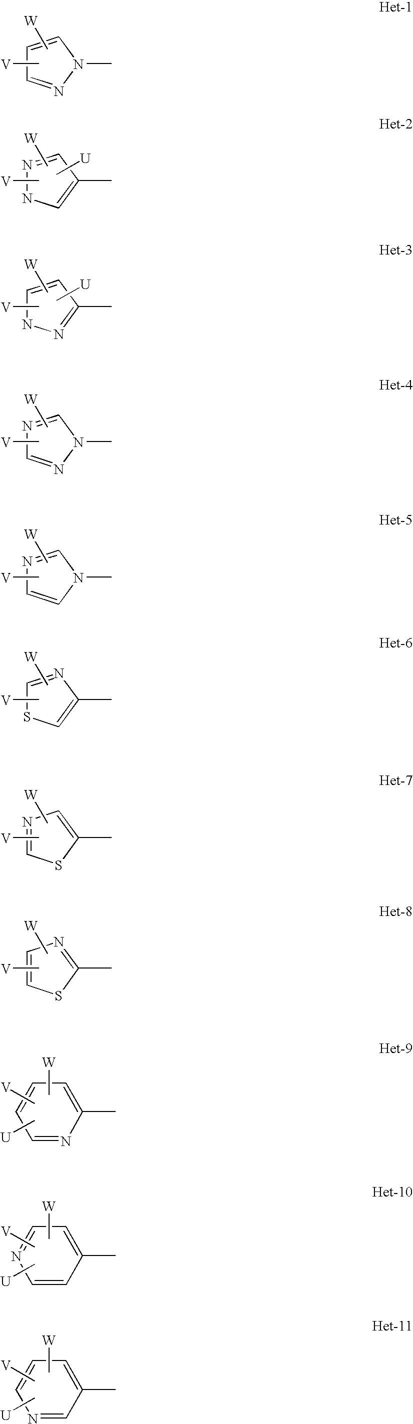 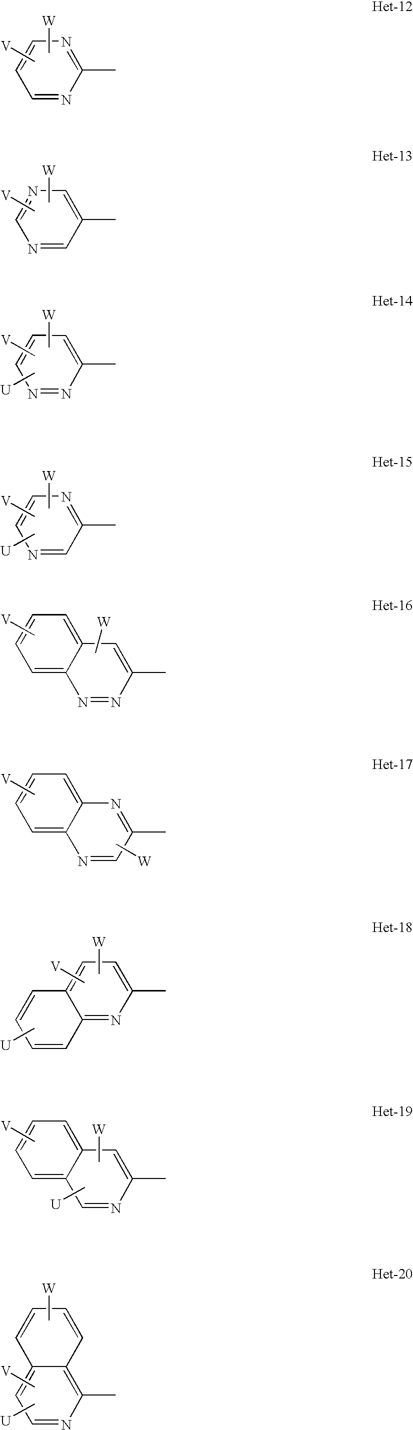 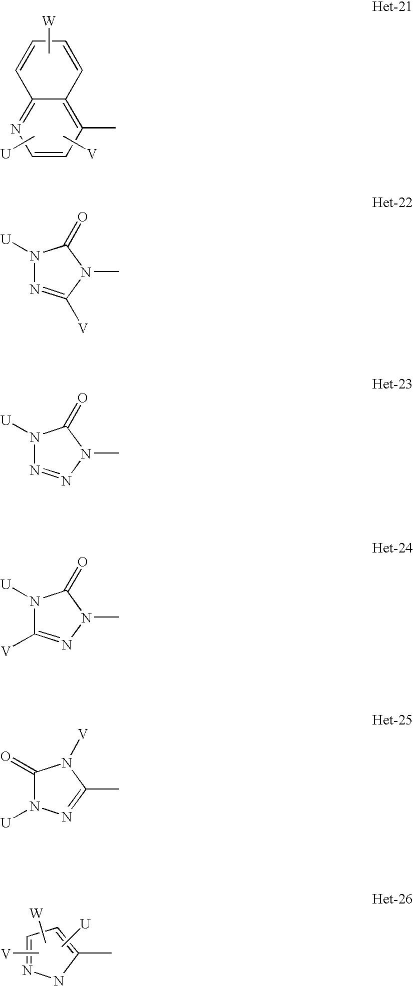
U, V and W independently of one another represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoron-propyl, carboxyl and/or carboxymethyl;
Z represents hydroxyl, halogen or in each case optionally substituted alkoxy, alkylthio, alkylsulphonyl or the group 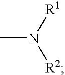
Y represents hydrogen, halogen, optionally substituted alkyl, haloalkyl, or optionally substituted cycloalkyl, O—(C₁-C₄)-alkyl or S(O)₀₋₂(C₁-C₄)-alkyl;
R¹ represents hydrogen, optionally substituted alkyl, optionally substituted alkenyl, optionally substituted alkynyl, haloalkyl, optionally substituted cycloalkyl or optionally substituted heterocyclyl, hydroxyl, optionally substituted alkoxy, amine, optionally substituted alkylamine or optionally substituted dialkylamine;
R² represents hydrogen or alkyl;
or
R¹ and R² together with the nitrogen atom to which they are attached represent an optionally substituted heterocyclic ring;
R³ represents 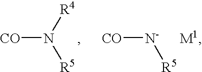
COOH, CO—O⁻M¹;
R⁴ represents hydrogen, in each case optionally substituted alkyl, alkenyl or alkynyl, haloalkyl, in each case optionally substituted saturated or unsaturated cycloalkyl which may optionally be interrupted by heteroatoms, in each case optionally substituted phenyl, heteroaryl, arylalkyl, heteroarylalkyl;
M¹ represents a cation, such as, for example, mono- or divalent metal atoms, or an optionally alkyl- or arylalkyl-substituted ammonium ion;
R⁵ represents the groups COR⁶, S(O)₁₋₂R⁶, cyano, COOR⁶; or
R⁴ and R⁵ together with the nitrogen atom to which they are attached represent an optionally substituted saturated, unsaturated or aromatic ring which may optionally be interrupted by further heteroatoms;
R⁶ represents hydrogen, in each case optionally substituted alkyl, alkenyl, alkynyl, haloalkyl, represents in each case optionally substituted cycloalkylalkyl, cycloalkyl or cycloalkenyl, in each case optionally substituted aryl, heteroaryl, arylalkyl or heteroarylalkyl;
or
R⁴ and R⁶ together with the N—CO or N—S(O)₁₋₂ group to which they are attached form a 4- to 8-membered cycle which may contain one or more heteroatoms from the group consisting of sulphur, oxygen and/or nitrogen, where oxygen atoms must not be adjacent;
and agrochemically active salts thereof.

2. Heterocyclic carboxylic acid derivatives of the formula (I) according to claim 1 in which the symbols have the following meaning:
X represents hydrogen, fluorine, chlorine, bromine, cyano, hydroxyl, alkoxy having 1 to 4 carbon atoms or alkythio having 1 to 4 carbon atoms, C₁-C₆-alkyl or C₁-C₄-haloalkyl having 1 to 9 halogen atoms;
Het represents one of the radicals 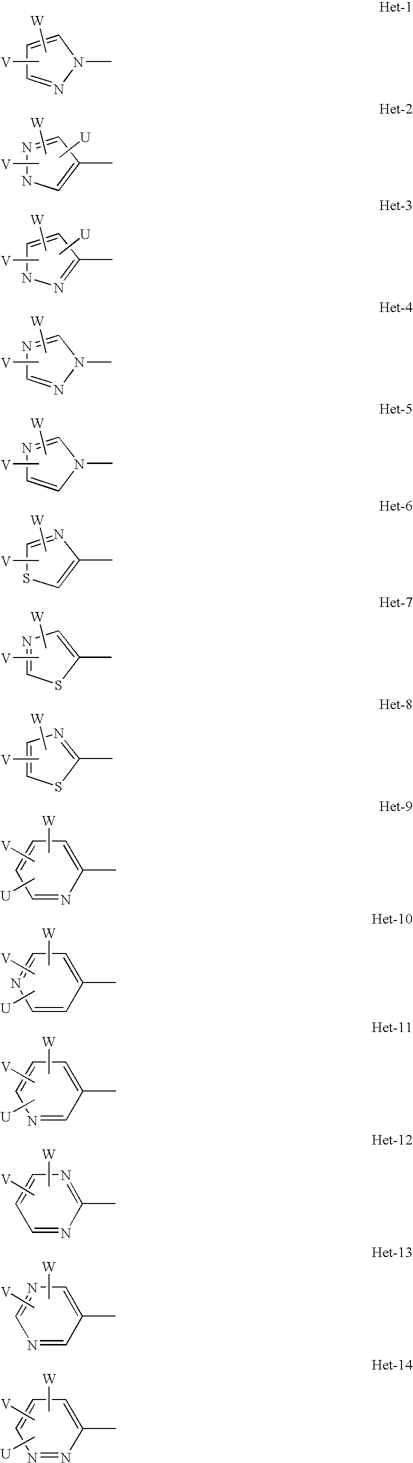 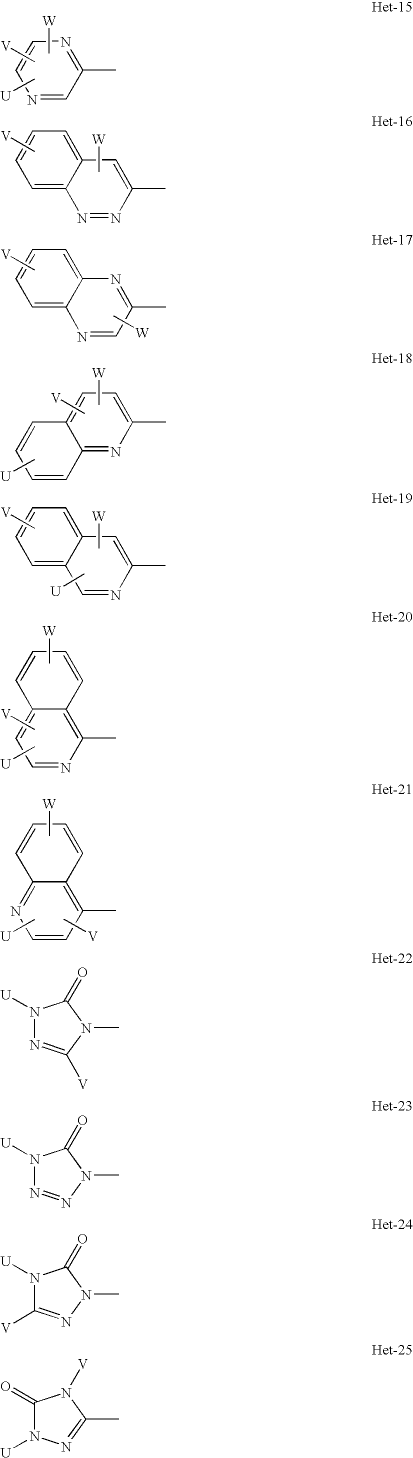 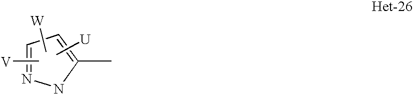
U, V and W independently of one another represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoron-propyl, carboxyl and/or carboxymethyl;
Z represents hydroxyl, chlorine, bromine, represents straight-chain or branched C₁-C₆-alkoxy, C₁-C₆-alkylthio, C₁-C₆-alkylsulphonyl, each of which is optionally substituted by 1 to 7 halogen atoms, or represents the group 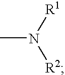
Y represents hydrogen, halogen, C₁-C₄-alkyl which is optionally substituted by 1 to 9 halogen atoms, C₃-C₆-cycloalkyl which is optionally substituted by 1 to 9 halogen atoms, SCH₃, SC₂H₅, SOCH₃, SOC₂H₅, SO₂CH₃, SO₂C₂H₅, OCH₃ or OC₂H₅;
R¹ represents hydrogen, alkyl having 1 to 10 carbon atoms which is unsubstituted or mono- to pentasubstituted by identical or different substituents from the group consisting of cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ represents alkenyl having 3 to 10 carbon atoms which is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ represents alkynyl having 3 to 10 carbon atoms which is unsubstituted or mono- to trisubstituted by identical or different substituents from the group consisting of halogen, cyano, hydroxyl, alkoxy having 1 to 4 carbon atoms, cycloalkyl having 3 to 6 carbon atoms, mercapto, alkylthio having 1 to 4 carbon atoms, amino, mono- or dialkylamino having in each case 1 to 4 carbon atoms; or
R¹ represents haloalkyl having 1 to 10 carbon and 1 to 21 halogen atoms; or
R¹ represents cycloalkyl having 3 to 10 carbon atoms which is unsubstituted or mono-to trisubstituted by identical or different substituents from the group consisting of halogen and alkyl, haloalkyl having in each case 1 to 4 carbon atoms; or
R¹ represents saturated or unsaturated heterocyclyl having 3 to 10 ring members and 1 to 3 heteroatoms, such as nitrogen, oxygen and/or sulphur, where the heterocyclyl is unsubstituted or mono- or polysubstituted by halogen, alkyl having 1 to 4 carbon atoms, alkoxyl having 1 to 4 carbon atoms, cyano, nitro, cycloalkyl having 3 to 6 carbon atoms, hydroxyl, alkoxy, alkenyloxy, alkynyloxy having 1 to 6 carbon atoms or mercapto;
R² represents hydrogen or alkyl having 1 to 6 carbon atoms; or
R¹ and R² together with the nitrogen atom to which they are attached represent a saturated or unsaturated heterocyclic ring having 3 to 8 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 4 carbon atoms, haloalkyl having 1 to 4 carbon atoms and 1 to 9 fluorine and/or chlorine atoms, hydroxyl, alkoxy having 1 to 4 carbon atoms, haloalkoxy having 1 to 4 carbon atoms and 1 to 9 fluorine and/or chlorine atoms, mercapto, thioalkyl having 1 to 4 carbon atoms and/or haloalkylthio having 1 to 4 carbon atoms and 1 to 9 fluorine and/or chlorine atoms;
R³ represents 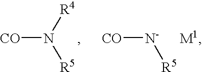
COOH, CO—O⁻M¹;
R⁴ represents hydrogen,
represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—O—C₁-C₄-alkyl, represents C₃-C₈-cycloalkyl which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄alkoxy, C₁-C₄-haloalkoxy, nitro or cyano;
M¹ represents cations from the group of the alkali metals or alkaline earth metals or ammonium ions NH₄, mono-(C₁-C₁₀)-alkylammonium, di-(C₁-C₁₀)-alkylammonium, tri-(C₁-C₁₀)-alkylammonium, tetra-(C₁-C₁₀)-alkylammonium, where the alkyl radicals of the ammonium ions may be substituted by aryl or hydroxyl, or cholinium;
R⁵ represents the groups CO—R⁶, S(O)₁₋₂R⁶, cyano, COOR⁶;
R⁴ and R⁵ together with the nitrogen atom to which they are attached represent a saturated or unsaturated 5- or 6-membered ring which is optionally mono- or polysubstituted by C₁-C₄-alkyl and which may optionally be interrupted by oxygen or sulphur;
R⁶ represents hydrogen, represents C₁-C₁₀-alkyl, C₃-C₁₀-alkenyl or C₃-C₁₀-alkynyl, each of which is optionally mono- or polysubstituted by fluorine, chlorine, C₁-C₄-alkoxy, C₁-C₄-alkylthio, cyano, CO₂—H or CO—O—C₁-C₄-alkyl, represents C₁-C₁₀-haloalkyl having 1 to 21 fluorine and/or chlorine atoms, represents C₃-C₈-cycloalkyl, C₃-C₆-cycloalkyl-C₁-C₂-alkyl or C₅-C₈-cycloalkenyl, each of which is optionally mono- or polysubstituted by fluorine, chlorine, C₁-C₄-alkyl, C₁-C₄-haloalkyl, C₁-C₄-alkoxy, CN, CO₂H or CO—O—C₁-C₄-alkyl and which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- to trisubstituted by fluorine, chlorine, bromine, C₁-C₄-alkyl, C₁-C₄-haloalkyl, C₁-C₄-alkoxy, C₁-C₄-haloalkoxy, nitro or cyano;
or
R⁴ and R⁶ together with the N—CO or N—S(O)₁₋₂ group to which they are attached form a 4- to 8-membered cycle which may contain one or more heteroatoms from the group consisting of sulphur, oxygen and nitrogen, where oxygen atoms must not be adjacent.

3. Heterocyclic carboxylic acid derivatives of the formula (I) according to claim 1 or 2 in which the symbols have the following meaning:
X represents hydrogen, fluorine, chlorine, bromine, cyano, C₁-C₄-alkyl or C₁-C₂-haloalkyl which may be substituted by one to five fluorine and/or chlorine atoms;
Het represents one of the radicals 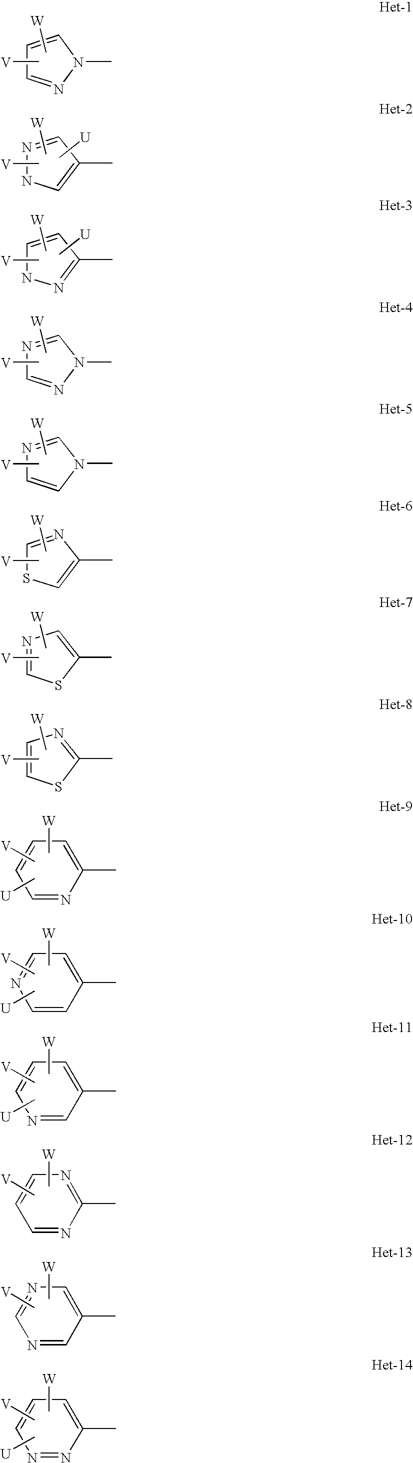 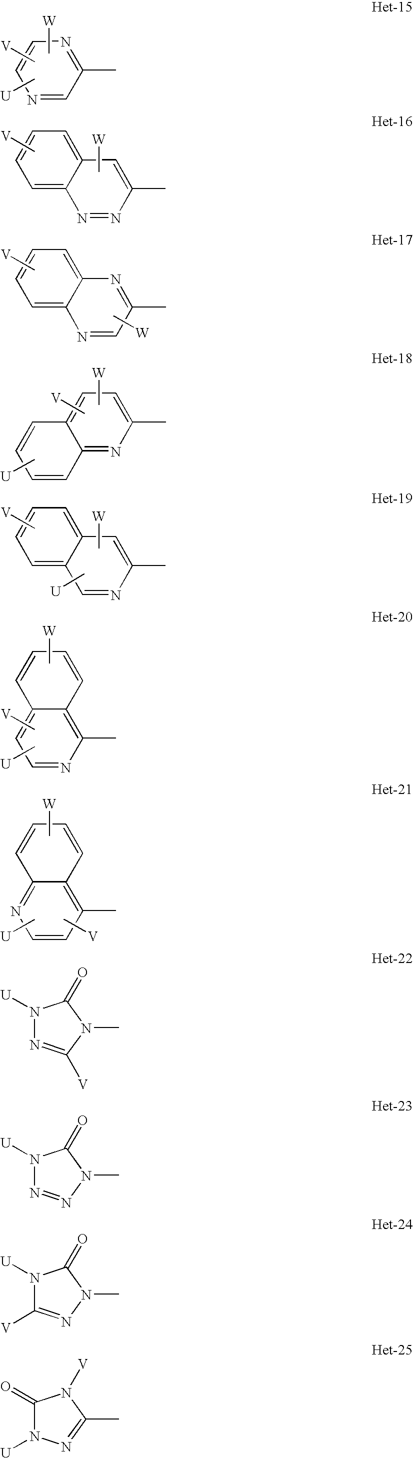 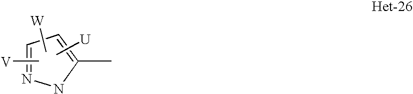
U, V and W independently of one another represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoron-propyl, carboxyl and/or carboxymethyl;
Z represents chlorine, bromine, represents straight-chain or branched C₁-C₄-alkoxy, C₁-C₄-alkylthio, each of which is optionally substituted by 1 to 3 halogen atoms, or represents the group 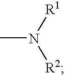
Y represents hydrogen, halogen, (C₁-C₄)-alkyl which is optionally substituted by 1 to 9 halogen atoms, cyclopropyl which is optionally substituted by 1 to 5 halogen atoms; SCH₃, SC₂H₅, SOCH₃, SOC₂H₅, SO₂CH₃, SO₂C₂H₅, OCH₃ or OC₂H₅;
R¹ represents hydrogen, alkyl having 1 to 8 carbon atoms which is unsubstituted or mono- to trisubstituted by identical or different substituents from the group consisting of cyano, alkoxy having 1 to 3 carbon atoms, cycloalkyl having 3 to 6 carbon atoms, alkylthio having 1 to 3 carbon atoms; or
R¹ represents alkenyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ represents alkynyl having 3 to 8 carbon atoms which is unsubstituted or mono- to trisubstituted by identical or different substituents from the group consisting of fluorine, chlorine, cyano, alkoxy having 1 to 3 carbon atoms, cycloalkyl having 3 to 6 carbon atoms, alkylthio having 1 to 3 carbon atoms; or
R¹ represents cycloalkyl having 3 to 8 carbon atoms which is unsubstituted or mono-to trisubstituted by identical or different substituents from the group consisting of fluorine, chlorine and alkyl, haloalkyl, alkoxy having in each case 1 or 2 carbon atoms; or
R¹ represents haloalkyl having 1 to 8 carbon and 1 to 17 fluorine and/or chlorine atoms; or
R¹ represents saturated or unsaturated heterocyclyl having 3 to 8 ring members and 1 or 2 heteroatoms, such as nitrogen, oxygen and/or sulphur, where the heterocyclyl is unsubstituted or mono- or disubstituted by hydroxyl, fluorine, chlorine, alkyl having 1 to 3 carbon atoms, alkoxy having 1 to 3 carbon atoms, cyano, nitro or cycloalkyl having 3 to 6 carbon atoms;
R² represents hydrogen or alkyl having 1 to 6 carbon atoms; or
R¹ and R² together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to trisubstituted by fluorine, chlorine, bromine, alkyl having 1 to 3 carbon atoms, haloalkyl having 1 to 3 carbon atoms and 1 to 5 fluorine and/or chlorine atoms, alkoxy having 1 to 3 carbon atoms, haloalkoxy having 1 to 3 carbon atoms and 1 to 5 fluorine and/or chlorine atoms, thioalkyl having 1 to 3 carbon atoms and/or haloalkylthio having 1 to 3 carbon atoms and 1 to 5 fluorine and/or chlorine atoms;
R³ represents 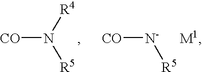
COOH, CO—O⁻M¹;
R⁴ represents hydrogen,
represents C₁-C₈-alkyl, C₃-C₈-alkenyl or C₃-C₈-alkynyl, each of which is optionally mono- to trisubstituted by fluorine, chlorine, C₁-C₃-alkoxy, C₁-C₃-alkylthio, cyano, CO₂H or CO—O—C₁-C₃-alkyl, represents C₃-C₆-cycloalkyl, C₃-C₆-cycloalkylmethyl, each of which is optionally mono- to trisubstituted by fluorine, chlorine, C₁-C₃-alkyl, C₁-C₂-haloalkyl, C₁-C₃-alkoxy, CN, CO₂H or CO—O—C₁-C₃-alkyl and which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
M¹ represents Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺, N(CH₃)₃—H₂—C₆H₅⁺, 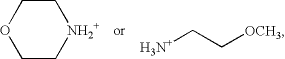
R⁵ represents the groups COR⁶, S(O)₁₋₂R⁶, cyano, COOR⁶;
R⁴ and R⁵ together with the nitrogen atom to which they are attached represent a saturated or unsaturated 5- or 6-membered ring which is optionally mono- or disubstituted by C₁-C₂-alkyl and which may optionally be interrupted by oxygen or sulphur;
R⁶ represents hydrogen, represents C₁-C₈-alkyl, C₃-C_{g}-alkenyl or C₃-C₈-alkynyl, each of which is optionally mono- to pentasubstituted by fluorine, chlorine, C₁-C₃-alkoxy, C₁-C₃-alkylthio, cyano, CO₂H or CO—O—C₁-C₃-alkyl, represents C₁-C₈-haloalkyl having 1 to 17 fluorine and/or chlorine atoms, represents C₃-C₆-cycloalkyl, C₃-C₆-cycloalkylmethyl or C₅-C₆-cycloalkenyl, each of which is optionally mono- to trisubstituted by fluorine, chlorine, C₁-C₄-alkyl, C₁-C₂-haloalkyl, C₁-C₂-alkoxy, CN, CO₂H or CO—O—C₁-C₂-alkyl and which may optionally be interrupted by an oxygen or sulphur atom,
represents phenyl, pyridyl, thiazolyl, pyrimidyl, benzyl, phenethyl, pyridinylmethyl or thiazolylmethyl, each of which is optionally mono- or disubstituted by fluorine, chlorine, bromine, C₁-C₃-alkyl, C₁-C₃-haloalkyl, C₁-C₃-alkoxy, C₁-C₃-haloalkoxy, nitro or cyano;
or
R⁴ and R⁶ together with the N—CO or N—S(O)₁₋₂ group to which they are attached form a 4- to 6-membered cycle which may contain one or more heteroatoms from the group consisting of sulphur, oxygen and nitrogen, where oxygen atoms must not be adjacent.

4. Heterocyclic carboxylic acid derivatives of the formula (I) according to any of claims 1 to 3, in which the symbols have the following meanings:
X represents hydrogen, chlorine, bromine, methyl or trifluoromethyl;
Het represents one of the radicals 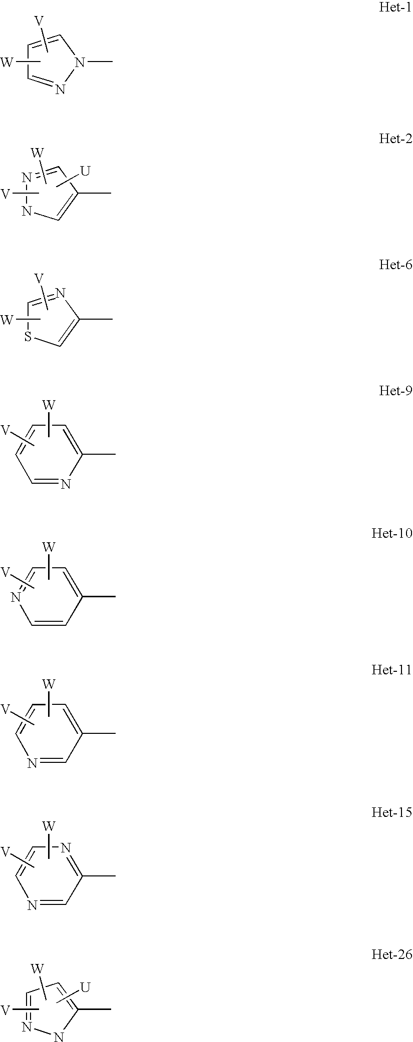
U, V and W independently of one another represent hydrogen, fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoron-propyl, carboxyl and/or carboxymethyl;
Z represents chlorine, methoxy, ethoxy, methylthio, ethylhio or the group 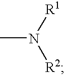
Y represents hydrogen, chlorine, bromine, alkyl having 1 or 2 carbon atoms, haloalkyl having 1 or 2 carbon and 1 to 5 halogen atoms or cyclopropyl;
R¹ represents hydrogen, alkyl having 1 to 4 carbon atoms which is unsubstituted or mono- to tetrasubstituted by identical or different substituents from the group consisting of methyl, ethyl, methoxy, ethoxy, cyclopropyl, cyclopentyl, cyclohexyl, methylthio and ethylthio; or
R¹ represents alkenyl having 3 to 6 carbon atoms which is unsubstituted or mono- to trisubstituted by identical or different substituents from the group consisting of fluorine, chlorine, methoxy, ethoxy, methylthio and ethylthio; or
R¹ represents alkynyl having 3 to 6 carbon atoms which is unsubstituted or mono- to trisubstituted by identical or different substituents from the group consisting of fluorine, chlorine, methoxy, ethoxy, methylthio and ethylthio; or
R¹ represents cycloalkyl having 3 to 6 carbon atoms which is unsubstituted or mono-to trisubstituted by identical or different substituents from the group consisting of fluorine, chlorine, methyl, ethyl, methoxy and trifluoromethyl;
R¹ represents haloalkyl having 1 to 4 carbon atoms which is substituted by 1 to 9 fluorine and/or chlorine atoms;
R² represents hydrogen, alkyl having 1 or 2 carbon atoms; or
R¹ and R² together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ represents 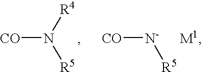
COOH, CO—O⁻M¹;
R⁴ represents hydrogen;
M¹ represents Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺;
R⁵ represents the groups COR⁶, S(O)R⁶, S(O)₂R⁶, cyano, COOR⁶;
R⁶ represents hydrogen, represents alkyl having 1 to 3 carbon atoms or cyclopropyl, each of which is optionally mono- to trisubstituted by methoxy, ethoxy, methylthio, ethylthio, cyano, CO—O-methyl or CO—O-ethyl, represents haloalkyl having 1 to 3 carbon and 1 to 7 fluorine and/or chlorine atoms.

5. Heterocyclic carboxylic acid derivatives of the formula (I) according to any of claims 1 to 4 in which the symbols are as defined below:
X represents hydrogen, chlorine, bromine, methyl or trifluoromethyl;
Het represents pyridinyl which is attached in the 2-, 3- or 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
represents pyrazinyl which is attached in the 2-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
represents pyrazolyl which is attached in the 1-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, trichloromethyl, pentafluoroethyl, heptafluoroisopropyl, heptafluoro-n-propyl, carboxyl and carboxymethyl or
represents pyrazolyl which is attached in the 4- or 5-position and may be mono- to trisubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl or
represents thiazolyl which is attached in the 4-position and may be mono- or disubstituted by identical or different substituents from the group consisting of fluorine, chlorine, bromine, cyano, hydroxyl, mercapto, nitro, methyl, ethyl, methoxy, methylthio, hydroximinomethyl, hydroximinoethyl, methoximinomethyl, methoximinoethyl, trifluoromethyl, carboxyl and carboxymethyl;
Z represents chlorine, methoxy, ethoxy, methylthio, ethylhio or the group 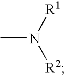
Y represents hydrogen, chlorine, bromine, methyl, ethyl, trifluoromethyl or cyclopropyl;
R¹ represents hydrogen, alkyl having 1 to 4 carbon atoms which is unsubstituted or mono- to tetrasubstituted by identical or different substituents from the group consisting of methyl, ethyl, methoxy, ethoxy, cyclopropyl, cyclopentyl, cyclohexyl, methylthio and ethylthio; or
R¹ represents alkenyl having 3 to 6 carbon atoms which is unsubstituted or mono- to trisubstituted by identical or different substituents from the group consisting of fluorine, chlorine, methoxy, ethoxy, methylthio and ethylthio; or
R¹ represents alkynyl having 3 to 6 carbon atoms which is unsubstituted or mono- to trisubstituted by identical or different substituents from the group consisting of fluorine, chlorine, methoxy, ethoxy, methylthio and ethylthio; or
R¹ represents cycloalkyl having 3 to 6 carbon atoms which is unsubstituted or mono-to trisubstituted by identical or different substituents from the group consisting of fluorine, chlorine, methyl, ethyl, methoxy and trifluoromethyl;
R¹ represents haloalkyl having 1 to 4 carbon atoms which is substituted by 1 to 9 fluorine and/or chlorine atoms;
R² represents hydrogen, methyl or ethyl; or
R¹ and R² together with the nitrogen atom to which they are attached represent a saturated or unsaturated heterocyclic ring having 3 to 6 ring members, where the heterocycle optionally contains a further nitrogen, oxygen or sulphur atom as ring member and where the heterocycle may be unsubstituted or up to disubstituted by fluorine, chlorine, methyl, ethyl, trifluoromethyl, methoxy or ethoxy;
R³ represents 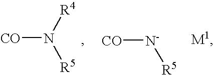
COOH, CO—O⁻M¹;
R⁴ represents hydrogen;
M¹ represents Na⁺, K⁺, ½ Ca²⁺, ½ Mg²⁺, NH₄⁺, NH₃CH₃⁺, NH₂(CH₃)₂⁺, NH(CH₃)₃⁺, NH(C₂H₅)₃⁺, NH₂(C₂H₅)₂⁺, NH₃C₂H₅⁺, NH₃i-C₃H₇⁺, NH₂(i-C₃H₇)₂⁺, NH₃—CH₂—C₆H₅⁺ or N(CH₃)₃—H₂—C₆H₅⁺;
R⁵ represents S(O)R⁶, S(O)₂R⁶,
R⁶ represents hydrogen, represents alkyl having 1 to 3 carbon atoms or cyclopropyl, each of which is optionally mono- to trisubstituted by fluorine, chlorine, methoxy, ethoxy, methylthio, ethylthio, cyano, CO—O-methyl or CO—O-ethyl, represents haloalkyl having 1-3 carbon and 1 to 7 fluorine and/or chlorine atoms.

6. Process for preparing heterocyclic carboxylic acid derivatives of the general formula (I), characterized in that
(A) compounds of the formula (II) 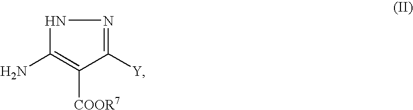
in which R⁷ represents C₁-C₄-alkyl and
Y has the meanings given in claim 1
are reacted with compounds of the formula (III) 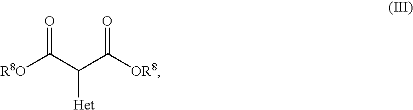
in which R⁸ represents C₁-C₈-alkyl or aryl and
Het has the meanings given in claim 1,
to give compounds of the formula (IV) 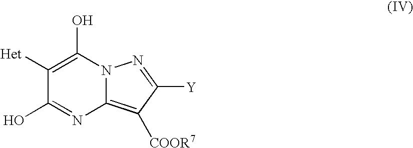
and
(B) the compounds of the formula (IV), as defined above, are then converted into compounds of the formula (V) 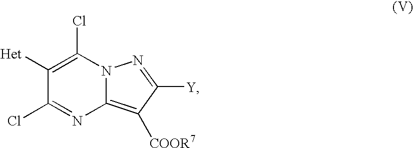
in which R⁷ represents C₁-C₄-alkyl and
Het and Y have the meanings given in claim 1,
and
(C) the compounds of the formula (V), as defined above, are then reacted with compounds of the formula HNR¹R², in which R¹ and R² are as defined in claim 1, to give compounds of the formula (VI) 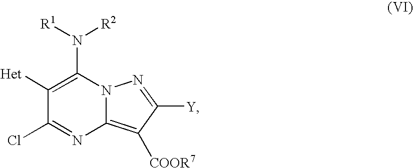
in which R⁷ represents C₁-C₄-alkyl and
R¹, R², Het and Y have the meanings given in claim 1,
and
(D) the compounds of the formula (VI), as defined above, are then converted in the presence of base into compounds of the formula (I-a) 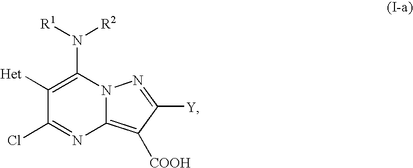
in which R¹, R², Het and Y have the meanings given in claim 1, and
(E) the compounds of the formula (I-a), as defined above, are then converted into compounds of the formula (VII) 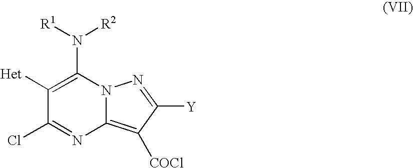
in which R¹, R², Het and Y have the meanings given in claim 1, and
(F) the compounds of the formula (VII), as defined above, are then reacted with compounds of the formula (VIII) 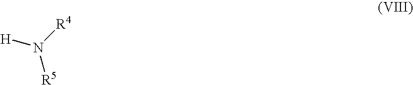
in which R⁴ and R⁵ have the meanings given in claim 1, to give compounds of the formula (I-b) 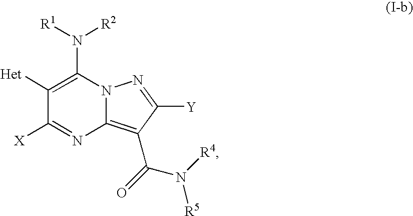
in which R¹, R², R⁴, R⁵, Het and Y have the meanings given in claim 1.

7. Mixture, comprising at least one of the compounds defined in any of claims 1 to 5 and/or at least one of the agrochemically active salts thereof and a further active compound selected from the group consisting of insecticides, attractants, sterilants, bactericides, acaricides, nematicides, fungicides, growth-regulators, herbicides, safeners, fertilizers and semiochemicals for controlling animal pests and/or phytopathogenic harmful fungi.

8. Use according to any of claims 1 to 5 and 7 for controlling animal pests and phytopathogenic harmful fungi.

9. Use according to any of claims 1 to 5 and 7 and 8 for treating plants or for treating seed of plants.

10. Use according to claim 9, where the plants are selected from the group of the transgenic plants.

11. Composition for controlling animal pests and/or phytopathogenic harmful fungi in and/or on plants or in and/or on seed of plants, where the composition mentioned comprises at least one compound defined in any of claims 1 to 5 and/or at least one of its agrochemically active salts or a mixture as defined in claim 7 and agrochemically customary auxiliaries and/or additives.

12. Composition according to claim 11 where the auxiliaries and/or additives are selected from the group consisting of extenders and/or surfactants.

13. Composition for controlling animal pests in and/or on plants or in and/or on seed of plants, where the composition mentioned comprises at least one compound as defined in any of claims 1 to 6 and/or at least one of its agrochemically active salts and at least one salt of the formula (XV) 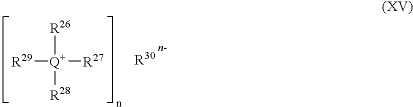
in which
Q represents nitrogen or phosphorus,
R²⁶, R²⁷, R²⁸ and R²⁹ independently of one another represent hydrogen or in each case optionally substituted C₁-C₈-alkyl or mono- or polyunsaturated, optionally substituted C₁-C₈-alkylene, where the substituents may be selected from the group consisting of halogen, nitro and cyano,
n represents 1, 2, 3 or 4,
R³⁰ represents an inorganic or organic anion.

14. Composition according to one or more of claims 11 to 13, characterized in that Q represents nitrogen.

15. Composition according to claim 14, characterized in that R³⁰ represents bicarbonate, tetraborate, fluoride, bromide, iodide, chloride, monohydrogenphosphate, dihydrogenphosphate, hydrogensulphate, tartrate, sulphate, nitrate, thiosulphate, thiocyanate, formate, lactate, acetate, propionate, butyrate, pentanoate, citrate or oxalate.

16. Composition according to any of claims 13 to 15, characterized in that it comprises at least one penetrant.

17. Method for controlling animal pests in and/or on plants or in and/or on seed of plants, which comprises bringing the pests mentioned directly or indirectly into contact with at least one compound as defined in any of claims 1 to 5 and/or at least one of the agrochemically active salts thereof or a mixture as defined in claim 7 or a composition as defined in any of claims 11 to 16.

18. Seed treated with at least one compound as defined in any of claims 1 to 5 and/or at least one of its agrochemically active salts or a mixture as defined in claim 7 or a composition as defined in any of claims 11 to 16.

19. Malonic esters of the formula (III) 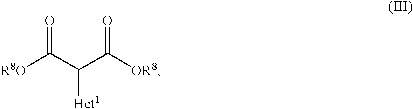
in which
R⁸ has the meaning given in claim 6 and
Het¹ is pyrazolyl which is attached in the 1-position and may be mono- or disubstituted by identical or different substituents from the group consisting of haloalkyl having 1 to 7 fluorine and/or chlorine atoms, in particular trifluoromethyl, trichloromethyl, pentafluoroethyl, heptafluoroisopropyl and heptafluoro-n-propyl, or
represents 4-chloropyrazol-1-yl.

20. Process for preparing malonic esters of the formula (III) as defined in claim 19, characterized in that heterocyclylacetic esters of the formula (IX) 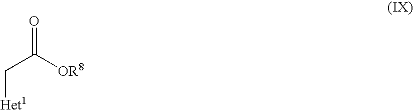
are reacted with compounds of the formula (X) 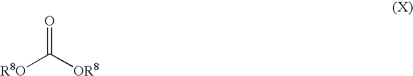
in the presence of base.
